(19) United States
(12) Patent Application Publication
(10) Pub. No.: US 2014/0039052 A1
(43) Pub. Date: Feb. 6, 2014

(54) FORMULATIONS OF LIPOPHILIC BIOACTIVE MOLECULES

(71) Applicants: Zumes, LLC, Hasbrouck Heights, NJ (US); National Research Council of Canada, Ottawa (CA)

(72) Inventors: Henryk Borowy-Borowski, Ottawa (CA); Volker Berl, New York, NY (US)

(21) Appl. No.: 13/804,584

(22) Filed: Mar. 14, 2013

Related U.S. Application Data
(63) Continuation of application No. 12/024,936, filed on Feb. 1, 2008, now abandoned.
(60) Provisional application No. 60/887,754, filed on Feb. 1, 2007.
(60) Provisional application No. 60/947,943, filed on Jul. 3, 2007.

(51) Int. Cl. A61K 31/202

(52) U.S. Cl. CPC A61K 31/202
USPC 514/560

(57) ABSTRACT

This invention provides aqueous and non-aqueous clear formulations including at least one lipophilic bioactive molecules and an amphiphilic solubilizing agent. Exemplary aqueous formulations include a water-soluble reducing agent, which diminishes or prevents chemical degradation of the lipophilic bioactive molecule. The invention also provides methods of using the formulations of the invention. For example, the invention provides beverages including the formulations of the invention. The invention further provides methods of making the formulations and beverages.

### CROSS-REFERENCES TO RELATED APPLICATIONS

This application is a continuation application of U.S. application Ser. No. 12/024,936, filed on Feb. 1, 2008, now abandoned, which claims priority under 35 U.S.C. §119(e) to U.S. Provisional Patent Application No. 60/887,754, filed on Feb. 1, 2007 and U.S. Provisional Patent Application No. 60/947,943, filed on Jul. 3, 2007, the disclosures of which are incorporated by reference herein in their entirety for all purposes.

### BACKGROUND OF THE INVENTION

A need exists in the art for improved formulations of lipophilic bioactive molecules that can be stored and subsequently used to prepare foods and beverages, pharmaceuticals and nutraceuticals, as well as skin-care and other consumer products. For example, a need exists for methods for chemically stabilizing lipophilic bioactive molecules in aqueous solutions. The current invention addresses these and other needs.

### SUMMARY OF THE INVENTION

In one aspect, the invention provides a water-soluble formulation comprising a lipophilic bioactive molecule, a water-soluble reducing agent and a solubilizing agent having a structure according to Formula (IV):

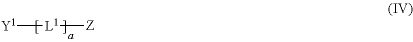

wherein a is an integer selected from 0 and 1; Z is a member selected from a sterol, a tocopherol, a ubiquinol and derivatives or homologues thereof; Y¹ is a linear or branched hydrophilic moiety comprising at least one polymeric moiety, wherein each of said polymeric moiety is a member independently selected from poly(alkylene oxides) and polyalcohols; and L¹ is a linker moiety selected from substituted or unsubstituted alkyl, substituted or unsubstituted heteroalkyl, substituted or unsubstituted aryl, substituted or unsubstituted heteroaryl and substituted or unsubstituted heterocycloalkyl. In one example, the lipophilic bioactive molecule is ubiquinol and the ubiquinol formulation is essentially free of ubiquinone. In one embodiment, the invention provides a beverage including the above ubiquinol formulation. Hence, in another aspect, the invention provides a non-alcoholic beverage including solubilized ubiquinol, a water-soluble reducing agent and a solubilizing agent having a structure according to Formula (IV).

In yet another aspect, the invention provides a non-alcoholic beverage comprising solubilized ubiquinone and a solubilizing agent of the invention. In one example, the solubilizing agent has a structure according to Formula (IV). In another example, the solubilizing agent is a member selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof. In yet another example, the ubiquinone beverage is essentially clear. For example, the ubiquinone beverage has a turbidity that is essentially stable for a period of at least 75 days when stored at an elevated temperature not exceeding about 90° F.

In a further aspect, the invention provides a process for making a water-soluble ubiquinol stock solution. The process includes contacting an emulsion of ubiquinone in an aqueous medium with an amount of a water-soluble reducing agent sufficient to essentially quantitatively reduce the ubiquinone to ubiquinol. The ubiquinone is solubilized in the above emulsion using a solubilizing agent having a formula according to Formula (IV). In an exemplary embodiment, the ubiquinone is CoQ₁₀ and the ubiquinol is ubiquinol-50.

In a further aspect, the invention provides a method for chemically stabilizing a lipophilic bioactive molecule in an aqueous formulation. The method includes contacting the lipophilic bioactive molecule with a ubiquinol stock solution of the invention (e.g., ubiquinol-50 stock solution).

In another aspect, the invention provides a process for the production of a non-alcoholic beverage. The process includes contacting an emulsion of ubiquinone in an aqueous medium with an original beverage. The ubiquinone is solubilized in the above emulsion using a solubilizing agent having a formula according to Formula (IV). The process can further include forming the emulsion of ubiquinone in an aqueous medium using a solubilizing agent having a structure according to Formula (IV).

In a further aspect, the invention provides a method for chemically stabilizing a lipophilic bioactive molecule in an aqueous formulation. The method includes contacting an emulsion of the lipophilic bioactive molecule in an aqueous medium with an amount of a water-soluble reducing agent sufficient to prevent chemical degradation of the molecule. The emulsion includes a solubilizing agent having a formula according to Formula (IV).

These and other aspects and advantages of the present invention will become apparent to those skilled in the art after considering the following detailed description of the invention.

### BRIEF DESCRIPTION OF THE DRAWINGS

FIG. 1 is a diagram representing the result of a dynamic light scattering (DLS) measurement determining the median particle size of micelles in an aqueous CoQ₁₀/PTS emulsion formed according to General Procedure 2 of Example 2. The sample was diluted to a PTS concentration between about 0.01 and about 1 mM before measurement. The diagram indicates that the median particle size of the micelle population in the dilute CoQ₁₀/PTS formulation is less than about 100 nm and lies between about 20 nm and about 30 nm.

FIG. 2A-E is a table summarizing stability data for selected beverages of the invention, which were prepared using water-soluble ubiquinone (CoQ₁₀) and ubiquinol (ubiquinol-50) formulations of the invention. The beverages included the following concentrations of lipophilic bioactive molecule: Gatorade and Fruit₂O included either 128 mg PTS alone, 36 mg CoQ₁₀/128 mg PTS or 36 mg/128 mg ubiquinol per serving. Sugar-sweetened Cola (SS Cola), Diet Cola and Propel included either 27 mg PTS alone, 7 mg CoQ₁₀/27 mg PTS or 7 mg/27 mg ubiquinol per serving. Beverages were stored at ambient temperature and at elevated temperatures for the indicated amount of time and were then analyzed for pH, color (absorbance at 440 and 520 nm wavelength) and turbidity. Turbidity was determined using a nephelometer. The units of turbidity are Nephelometric Turbidity Units (NTU). Reference standards with known turbidity were used to measure the turbidity of each sample. Temperatures in FIG. 2A-E are given in ° F.

### DETAILED DESCRIPTION OF THE INVENTION

### I. Definitions

The term “vitamin C derivative” as used herein means any compound that releases ascorbic acid (vitamin C) in vivo or in vitro, as well as solvates, hydrates and salts thereof. The term also includes vitamin C analogs wherein one or more of the hydroxyl groups of vitamin C are substituted with another moiety and wherein the vitamin C analog essentially retains the stabilizing activity of vitamin C in vitro or in vivo.

The term “monoterpene” as used herein, refers to a compound having a 10-carbon skeleton with non-linear branches. A monoterpene refers to a compound with two isoprene units connected in a head-to-end manner. The term “monoterpene” is also intended to include “monoterpenoid”, which refers to a monoterpene-like substance and may be used loosely herein to refer collectively to monoterpenoid derivatives as well as monoterpenoid analogs. Monoterpenoids can therefore include monoterpenes, alcohols, ketones, aldehydes, ethers, acids, hydrocarbons without an oxygen functional group, and so forth.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “solubilizing agent” is used interchangeably with the term “surfactant”. Solubilizing agents of the invention include compounds having a structure according to Formula (III). In one embodiment, the solubilizing agent is a non-ionic, amphiphilic molecule, wherein the term amphiphilic means that the molecule includes at least one hydrophobic (e.g., lipid-soluble) moiety, such as a moiety derived from a tocopherol, a sterol or a ubiquinone and at least one hydrophilic (e.g., water-soluble) moiety, such as polyethylene glycol. Other hydrophobic and hydrophilic moieties of the invention are discussed herein in the context of Formula (III).

As used herein, the terms “stabilizer”, “antioxidant” are recognized in the art and refer to synthetic or natural substances that prevent or delay the oxidative or free radical or photo induced deterioration of a compound. Exemplary stabilizers include tocopherols, flavonoids, catechins, superoxide dismutase, lecithin, gamma oryzanol; vitamins, such as vitamins A, C (ascorbic acid) and E (tocopherol and tocopherol homologues and isomers, especially alpha and gamma-tocopherol) and beta-carotene; natural components such as camosol, carnosic acid and rosmanol found in rosemary and hawthorn extract, proanthocyanidins such as those found in grapeseed or pine bark extract, and green tea extract.

The term “reducing agent” is any compound capable of reducing a compound of the invention to its reduced form. “Reducing agent” includes lipophilic (e.g., lipid-soluble) reducing agents. In one example, the lipid-soluble reducing agent incorporates a hydrophobic moiety, such as a substituted or unsubstituted carbon chain (e.g., a carbon chain consisting of at least 10 carbon atoms). “Reducing agent” also includes hydrophilic (e.g., water-soluble) reducing agents.

In one example, the reducing agent is a “water-soluble reducing agent” when the reducing agent dissolves in water (e.g., at ambient temperature) to produce a clear solution, as opposed to a visibly cloudy, hazy or otherwise inhomogeneous mixture, or even a two phase system. In one example, the reducing agent is a “water-soluble reducing agent” when it includes at least one (e.g., at least two) hydroxyl group(s) and does not include a large hydrophobic moiety (e.g., a substituted or unsubstituted linear carbon chain consisting of more than 10, 11, 12, 13, 14, 15, 16, 17, 18, 19 or 20 carbon atoms). In another example, the reducing agent is a “water-soluble reducing agent” when it includes at least one (e.g., at least two) hydroxyl group(s) and includes a substituted or unsubstituted linear carbon chain consisting of not more 6, 8, 10, 11, 12, 13, 14, or 15 carbon atoms). An exemplary water-soluble reducing agent is ascorbic acid. The term “water-soluble reducing agent” also includes mixtures of vitamin C with a lipophilic bioactive molecule of the invention. For example, a ubiquinol stock solution of the invention is a water-soluble reducing agent. Water-soluble reducing agents can be derivatized to afford an essentially lipid-soluble reducing agent. For example, the water-soluble reducing agent is derivatized with a fatty acid to give, e.g., a fatty acid ester. An exemplary lipid-soluble reducing agent is ascorbic acid-palmitate.

The term “water-soluble” when referring to a formulation or compositions of the invention, means that the formulation when added to an aqueous medium (e.g., water, original beverage) dissolves in the aqueous medium to produce a solution that is essentially clear. In one example, the formulation dissolves in the aqueous medium without heating the resulting mixture above ambient temperature (e.g., 25° C.). The term “essentially clear” is defined herein.

The term “aqueous formulation” refers to a formulation of the invention including at least about 5% (w/w) water. In one example, an aqueous formulation includes at least about 10%, at least about 20%, at least about 30% at least about 40% or at least about 50% (w/w) of water.

The term “bioactive” refers to compounds and compositions of the invention. For example, a bioactive molecule is any compound having in vivo and/or in vitro biological activity. Bioactive molecules or compositions also include those, which are suspected in the art to have biological activity (e.g., to have a positive effect on human health and/or nutrition). In one example, the biological activity is a desirable biological activity but can be accompanied by undesirable side-effects. Compounds with biological activity include pharmaceuticals, neutraceuticals and dietary supplements.

As used herein, the term “pharmaceutical”, “pharmaceutical composition” or pharmaceutical formulation” encompasses “neutraceutical” (also referred to as “nutraceutical”), “neutraceutical composition” or “neutraceutical formulation”, respectively. Neutraceutical formulations or neutraceutical compositions may include a pharmaceutically acceptable carrier, such as those described herein.

The term “neutraceutical” or “nutraceutical” is a combination of the terms “nutritional” and “pharmaceutical”. It refers to a composition, which is known or suspected in the art to positively affect human nutrition and/or health.

The term “beverage” describes any water-based liquid, which is suitable for human consumption (i.e., food-grade). A typical beverage of the invention is any “original beverage” in combination with at least one bioactive lipophilic molecule of the invention. “Original beverage” can be any beverage (e.g., any marketed beverage). The term “original beverage” includes beers, carbonated and non-carbonated waters (e.g., table waters and mineral waters), flavored waters (e.g., fruit-flavored waters), mineralized waters, sports drinks (e.g., Gatorade), smoothies, neutraceutical drinks, filtered or non-filtered fruit and vegetable juices (e.g., apple juice, orange juice, cranberry juice, pineapple juice, lemonades and combinations thereof) including those juices prepared from concentrates. Exemplary juices include fruit juices having 100% fruit juice (squeezed or made from concentrate), fruit drinks (e.g., 0-29% juice), nectars (e.g., 30-99% juice). The term “original beverage” also includes fruit flavored beverages, carbonated drinks, such as soft-drinks, fruit-flavored carbonates and mixers. Soft drinks include caffeinated soft drinks, such as coke (e.g., Pepsi Cola, Coca Cola) and any “diet” versions thereof (e.g., including non-sugar sweeteners). The term “original beverage” also includes teas (e.g., green and black teas, herbal teas) including instant teas, coffee, including instant coffee, chocolate-based drinks, malt-based drinks, milk, drinkable dairy products and beer. The term “original beverage” also includes any liquid or powdered concentrates used to make beverages.

The term “clear beverage” (e.g., clear juice) means any beverage clear (e.g., transparent) to the human eye. Typical clear beverages include carbonated or non-carbonated waters, soft drinks, such as Sprite or Coke, filtered juices and filtered beers. Typical non-clear beverages include orange juice with pulp and milk.

The term “non-alcoholic beverage” includes beverages containing essentially no alcohol. Exemplary non-alcoholic beverages include those listed above for the term “beverage”. The term “non-alcoholic beverage” includes beers, including those generally referred to as “non-alcoholic beers”. In one example, the non-alcoholic beverage includes less than about 10% alcohol by volume. In another example, the non-alcoholic beverage includes less than about 9% or less than about 8% alcohol by volume. In yet another example, the non-alcoholic beverage includes less than about 7%, less than about 6% or less than about 5% alcohol by volume.

The term “essentially free of ubiquinone” refers to a water-soluble composition of the invention, in which the ratio of ubiquinone to corresponding ubiquinol content is less than about 10%. The ratio can be measured using chromatography, such as standard analytical HPLC, for example in combination with peak integration (e.g., AUC). In one example, “essentially free of ubiquinone” refers to a ratio of ubiquinone:ubiquinol of less than about 5%. In another example, “essentially free of ubiquinone” refers to a ratio of ubiquinone:ubiquinol of less than about 3%. In yet another example, “essentially free of ubiquinone” refers to a ratio of ubiquinone:ubiquinol of less than about 1%. In a further example, “essentially free of ubiquinone” refers to a ratio of ubiquinone:ubiquinol of less than about 0.5%, less than about 0.4%, less than about 0.3%, less than about 0.2% or less than about 0.1%. In one example, “essentially free of ubiquinone” means that the residual concentration of ubiquinone (e.g., CoQ₁₀) is below the detectable level when measured by standard analytical HPLC.

The term “essentially stable to chemical degradation” refers to a bioactive molecule of the invention as contained in a formulation (e.g., aqueous formulation), beverage or other composition of the invention. In one example, “essentially stable to chemical degradation” means that the molecule is stable in its original (e.g., reduced) form and is not converted to another species (e.g., oxidized species; any other species including more or less atoms; any other species having an essentially different molecular structure), for example, through oxidation, cleavage, rearrangement, polymerization and the like, including those processes induced by light (e.g., radical mechanisms). Chemical degradation does not include solvation, deprotonation of acidic compounds, protonation of basic compounds, tautomerization and the like. Examples of chemical degradation include oxidation of ubiquinols to ubiquinones, oxidation or cleavage of double bonds in unsaturated fatty acids and light-induced rearrangements of unsaturated molecules. For example, the molecule is essentially stable when the concentration of its original (e.g., reduced) form in the composition (e.g., aqueous formulation) is not significantly diminished over time. For example, the molecule is essentially stable when the concentration of the original form of the molecule remains at least 80% when compared with the concentration of the original form of the molecule at about the time when the composition was prepared. In another example, the molecule is essentially stable when the concentration of the original form remains at least about 85%, at least about 90% or at least about 95% of the original concentration. For example, an aqueous composition containing ubiquinol at a concentration of about 50 mg/ml is considered essentially stable for at least 90 days when, at the end of the 90 days, the concentration of ubiquinol in the aqueous composition remains at least about 40 mg/ml (80% of 50 mg/ml).

The term “essentially clear” is used herein to describe the compositions (e.g., formulations) of the invention. For example, the term “essentially clear” is used to describe an aqueous formulation or a beverage of the invention. In one example, clarity is assessed by the normal human eye. In this example, “essentially clear” means that the composition is transparent and essentially free of visible particles and/or precipitation (e.g., not visibly cloudy, hazy or otherwise non-homogenous). In another example, clarity, haziness or cloudiness of a composition is assessed using light scattering technology, such as dynamic light scattering (DLS), which is useful to measure the sizes of particles, e.g., micelles, contained in a composition. In one example, “essentially clear” means that the median particle size as measured by DLS is less than about 100 nm. For example, when the median particle size is less than 100 nm the liquid appears clear to the human eye. In another example, “essentially clear” means that the median particle size is less than about 80 nm. In yet another example, “essentially clear” means that the median particle size is less than about 60 nm. In a further example, “essentially clear” means that the median particle size is less than about 40 nm. In another example, “essentially clear” means that the median particle size is between about 20 and about 30 nm. A person of skill in the art will know how to prepare a sample for DLS measurement. For example, in order to prepare a sample (e.g., formulation of the invention) for a DLS measurement, the sample is typically diluted so that the concentration of the solubilizing agent in the diluted sample is between about 1 mM (10⁻³ M) and 0.01 mM (10⁻⁵ M). In another example, the solubilizing agent (e.g., PTS) is present in a concentration that is above the critical micelle concentration (CMC) (i.e., concentration that allows for spontaneous formation of micelles). For example, a typical CMC for PTS in water is about 0.1 to about 0.5 mg/ml. A person of skill in the art will be able to select suitable concentrations in order to successfully measure particle sizes in a formulation of the invention.

Alternatively, clarity, haziness or cloudiness of a composition of the invention can be determined by measuring the turbidity of the sample. This is especially useful when the composition is a beverage (e.g., water, soft-drink etc.). In one example, turbidity is measured in FTU (Formazin Turbidity Units) or FNU (Formazin Nephelometric Units). In one example, turbidity is measured using a nephelometer, known in the art. Nephelometric measurements are based on the light-scattering properties of particles. The units of turbidity from a calibrated nephelometer are called Nephelometric Turbidity Units (NTU). In one example, reference standards with known turbidity are used to measure the turbidity of a sample. In one example, a composition of the invention (e.g., a beverage of the invention) is “essentially clear” when the turbidity is not more than about 500% higher than the control (original beverage without an added lipophilic bioactive molecule of the invention, but optionally including a solubilizing agent of the invention, e.g. PTS). For example, the turbidity of a sample of flavored water is measured to be 2.0 ntu and the turbidity of another sample containing the same flavored water in combination with ubiquinol is measured to be at or below about 8.0 ntu (2.0 ntu+200%=8.0 ntu), then the ubiquinol sample is considered to be essentially clear. In another example, a composition of the invention is “essentially clear” when the turbidity is not more than about 300% higher than the control. In yet another example, a composition of the invention is “essentially clear” when the turbidity is not more than about 200%, about 150% or about 100% higher than the control. In a further example, a composition of the invention is “essentially clear” when the turbidity is not more than about 80%, about 60%, about 40%, about 20% or about 10% higher than the control.

The term “emulsion” as used herein refers to a lipophilic molecule of the invention emulsified (solubilized) in an aqueous medium using a solubilizing agent of the invention. In one example, the emulsion includes micelles formed between the lipophilic molecule(s) and the solubilizing agent. When those micelles are sufficiently small, the emulsion is essentially clear. Typically, the emulsion will appear clear (e.g., transparent) to the normal human eye, when those micelles have a median particle size of less than 100 nm. In one example, the micelles in the emulsions of the invention have median particle sizes below 60 nm. In a typical example, micelles formed in an emulsion of the invention have a median particle size between about 20 and about 30 nm. In another example, the emulsion is stable, which means that separation between the aqueous phase and the lipophilic component does essentially not occur (e.g., the emulsion stays clear). A typical aqueous medium, which is used in the emulsions of the invention, is water, which may optionally contain other solubilized molecules, such as salts, coloring agents, flavoring agents and the like. In one example, the aqueous medium of the emulsion does not include an alcoholic solvent, such as ethanol or methanol.

The term “micelle” is used herein according to its art-recognized meaning and includes all forms of micelles, including, for example, spherical micelles, cylindrical micelles, worm-like micelles and sheet-like micelles.

The term “flavonoid” as used herein is recognized in the art. The term “flavonoid” includes those plant pigments found in many foods that are thought to help protect the body from disease (e.g., cancer). These include, for example, epi-gallo catechin gallate (EGCG), epi-gallo catechin (EGC) and epi-catechin (EC).

The term “tocopherol” includes all tocopherols, including alpha-, beta-, gamma- and delta tocopherol. The term “tocopherol” also includes tocotrienols.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl,” by itself or as part of another substituent, means, unless otherwise stated, a straight or branched chain, or cyclic hydrocarbon radical, or combination thereof, which can be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl” with the difference that the heteroalkyl group, in order to qualify as an alkyl group, is linked to the remainder of the molecule through a carbon atom. Alkyl groups that are limited to hydrocarbon groups are termed “homoalkyl”.

The term “alkenyl” by itself or as part of another substituent is used in its conventional sense, and refers to a radical derived from an alkene, as exemplified, but not limited, by substituted or unsubstituted vinyl and substituted or unsubstituted propenyl. Typically, an alkenyl group will have from 1 to 24 carbon atoms, with those groups having from 1 to 10 carbon atoms being preferred.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, S, B and P and wherein the nitrogen and sulfur atoms can optionally be oxidized and the nitrogen heteroatom can optionally be quaternized. The heteroatom(s) can be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms can be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. A “cycloalkyl” or “heterocycloalkyl” substituent can be attached to the remainder of the molecule directly or through a linker. An exemplary linker is alkylene. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (e.g., from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, ═O, NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR″″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R″ and R″″ groups when more than one of these groups is present.

Two of the substituents on adjacent atoms of the aryl or heteroaryl ring can optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring can optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed can optionally be replaced with a double bond. Alternatively, two of the substituents on adjacent atoms of the aryl or heteroaryl ring can optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R″ are independently selected from hydrogen or substituted or unsubstituted (C₁-C₆)alkyl.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems can include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S), silicon (Si) and boron (B).

The symbol “R” is a general abbreviation that represents a substituent group. Exemplary substituent groups include substituted or unsubstituted alkyl, substituted or unsubstituted heteroalkyl, substituted or unsubstituted aryl, substituted or unsubstituted heteroaryl, and substituted or unsubstituted heterocycloalkyl groups.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts.

When a residue is defined as “O⁻”, the formula is meant to optionally include an organic or inorganic cationic counterion. For example, the resulting salt form of the compound is pharmaceutically acceptable.

The neutral forms of the compounds are, for example, regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention can exist in multiple crystalline or amorphous forms (“polymorphs”). In general, all physical forms are of use in the methods contemplated by the present invention and are intended to be within the scope of the present invention. “Compound or a pharmaceutically acceptable salt, hydrate, polymorph or solvate of a compound” intends the inclusive meaning of “or”, in that materials meeting more than one of the stated criteria are included, e.g., a material that is both a salt and a solvate is encompassed.

The compounds of the present invention can contain unnatural proportions of atomic isotopes at one or more of the atoms that constitute such compounds. For example, the compounds can be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Certain compounds of the present invention possess asymmetric carbon atoms (optical centers) or double bonds; the racemates, diastereomers, geometric isomers and individual isomers are encompassed within the scope of the present invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis- and trans-isomers, (−)- and (+)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

As used herein, the term “leaving group” refers to a portion of a substrate that is cleaved from the substrate in a reaction. The leaving group is an atom (or a group of atoms) that is displaced as stable species taking with it the bonding electrons. Typically the leaving group is an anion (e.g., Cl⁻) or a neutral molecule (e.g., H₂O). Exemplary leaving groups include a halogen, OC(O)R⁶⁵, OP(O)R⁶⁵R⁶⁶, OS(O)R⁶⁵, and OSO₂R⁶⁵. R⁶⁵ and R⁶⁶ are members independently selected from substituted or unsubstituted alkyl, substituted or unsubstituted aryl, substituted or unsubstituted heteroaryl and substituted or unsubstituted heterocycloalkyl. Useful leaving groups include, but are not limited to, other halides, sulfonic esters, oxonium ions, alkyl perchlorates, sulfonates, e.g., arylsulfonates, ammonioalkanesulfonate esters, and alkylfluorosulfonates, phosphates, carboxylic acid esters, carbonates, ethers, and fluorinated compounds (e.g., triflates, nonaflates, tresylates), S R⁶⁵, (R⁶⁵)₃P⁺, (R⁶⁵)₂S⁺, P(O)N(R⁶⁵)₂(R⁶⁵)₂, P(O)XR⁶⁵X′R⁶⁵ in which each R⁶⁵ is independently selected from the members provided in this paragraph and X and X′ are S or O. The choice of these and other leaving groups appropriate for a particular set of reaction conditions is within the abilities of those of skill in the art (see, for example, March J, ADVANCED ORGANIC CHEMISTRY, 2nd Edition, John Wiley and Sons, 1992; Sandler S R, Karo W, ORGANIC FUNCTIONAL GROUP PREPARATIONS, 2nd Edition, Academic Press, Inc., 1983; and Wade L G, COMPENDIUM OF ORGANIC SYNTHETIC METHODS, John Wiley and Sons, 1980).

“Protecting group,” as used herein refers to a portion of a substrate that is substantially stable under a particular reaction condition, but which is cleaved from the substrate under a different reaction condition. A protecting group can also be selected such that it participates in the direct oxidation of the aromatic ring component of the compounds of the invention. For examples of useful protecting groups, see, for example, Greene et al., PROTECTIVE GROUPS IN ORGANIC SYNTHESIS, 3rd ed., John Wiley & Sons, New York, 1999.

“Ring” as used herein means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 to 7 atoms in the encircling arrangement. The ring optionally includes a heteroatom. Thus, the term “5- to 7-membered ring” includes, for example pyridinyl and piperidinyl. The term “ring” further includes a ring system comprising more than one “ring”, wherein each “ring” is independently defined as above.

### II. Introduction

The current invention provides aqueous compositions including a lipophilic bioactive molecule (e.g., CoQ₁₀) and a solubilizing agent described herein. These formulations have several advantages. First, they provide a lipophilic bioactive molecules (e.g., a bioactive molecule that is normally essentially water-insoluble) in an essentially clear, aqueous solution. This formulation can enable a consumer to ingest the lipophilic bioactive molecule in a liquid form, for example, in a beverage, such as water. The aqueous formulations are essentially clear, which makes the formulations more appealing to a consumer.

In another embodiment, the current invention provides formulations (e.g., aqueous formulations) of lipophilic bioactive molecules (e.g., ubiquinol-50) that include a solubilizing agent described herein as well as a water-soluble reducing agent (also referred to as a stabilizer). The lipophilic bioactive molecules in these formulations (especially aqueous formulations) are surprisingly stable with respect to chemical degradation (e.g., oxidation). In one example, the chemical stability of the lipphilic compounds is a result of a synergistic effect between the nature of the solubilizing agent and the water-solubility of the reducing agent (stabilizer): The solubilizing agent is an amphiphilic, non-ionic surfactant, which in aqueous solutions allows the lipophilic molecule to be emulsified in “nano-micelles”, which typically have an average particle size of not more than 100 nm, often below 30 nm. When the lipophilic molecule is solubilized in the form of these small micelles, a water-soluble (as opposed to lipid-soluble) reducing agent is surprisingly effective in preventing chemical degradation of the lipophilic molecule in an aqueous solution. For example, the addition of a water-soluble reducing agent diminishes or prevents the degradation of the lipophilic bioactive molecule and extends its average lifetime in solution, for example by at least 5 times. Molecules that are vulnerable to oxidation in aqueous solutions include omega-3-fatty acids (e.g., DHA) and ubiquinol.

In another example, the water-soluble reducing agent itself can be a compound with potential health benefits (e.g., vitamin C and other vitamins). Hence, the combination of two beneficial ingredients (lipophilic bioactive molecule and stabilizer) in a single composition provides greater convenience to a consumer. In a further example, the lipophilic bioactive molecule is first reduced by the water-soluble reducing agent into a chemical form that is more bioavailable and the reduced form is subsequently stabilized by an excess of reducing agent.

The invention also provides a method for making an aqueous, water-soluble ubiquinol formulation of the invention. In one example, the ubiquinol formulation is essentially free of ubiquinone (e.g., at least 90% of the combined ubiquinol/ubiquinol content is ubiquinol). An exemplary process includes contacting an emulsion of ubiquinone (e.g., CoQ₁₀) in an aqueous medium (e.g., water) with an amount of a water-soluble reducing agent (e.g., vitamin C or a water-soluble derivative of vitamin C) that is sufficient to essentially quantitatively reduce the ubiquinone to ubiquinol (e.g., ubiquinol-50). In one example, the ubiquinone emulsion is formed using a solubilizing agent of the invention. In one example, the aqueous ubiquinol formulation thus formed is essentially clear.

The inventors have discovered that the above process, in which ubiquinol is formed in situ from solubilized (emulsified) ubiquinone is superior to a related process, in which pre-formed (isolated) ubiquinol is contacted with a solubilizing agent and an aqueous medium. The current process is has several advantages. First, the process starts with widely available ubiquinone, which is cheaper than isolated ubiquinol. Second, once the ubiquinol is formed in situ, the ubiquinol is stable in the aqueous solution. Third, the process does not rely on inert gas to produce a formulation that is essentially free of ubiquinone. Overall, the current process is more cost-effective and does not require sophisticated equipment.

The water-soluble formulations of the invention can be used to prepare beverages having a lipophilic bioactive molecule stably dissolved therein.

### III. Compositions

The present invention provides formulations of lipophilic bioactive molecules. These formulations comprise at least (a) a lipophilic bioactive molecule of the invention and (b) a solubilizing agent of the invention. Exemplary lipophilic bioactive molecules and solubilizing agents, which can be used in the formulations of the invention, are described herein below.

In one example, the formulation further comprises (c) a water-soluble reducing agent. The inventors have discovered that certain lipophilic bioactive molecules, which are normally prone to chemical degradation (e.g., oxidation) can be stabilized using a water-soluble reducing agent, when the molecule is formulated using a solubilizing agent of the invention (e.g., PTS, PSS, PCS or PQS). An exemplary water-soluble reducing agent is selected from ascorbic acid (vitamin C) and water-soluble derivatives of vitamin C. Vitamin C is a convenient reducing agent because it is widely available and suitable for human consumption.

The inventors have further discovered that water-soluble, polar reducing agents are superior to lipid-soluble reducing agents with respect to their capabilities to chemically stabilize lipophilic molecules in aqueous solutions. Hence, in one example, the reducing agent is not a lipid-soluble reducing agent, such as vitamin C-palmitate.

The invention further provides methods of making the formulations. The formulations of the invention can be used in a variety of products, such as foods, beverages, cosmetics and skin-care products (topical application), dietary supplements (e.g., formulated in soft-gelatine capsules) and nutraceuticals. In one embodiment, the invention provides a beverage including a formulation of the invention.

### Formulations

In one aspect, the invention provides a water-soluble formulation including at least one lipophilic bioactive molecule, a water-soluble reducing agent and a solubilizing agent of the invention. Exemplary solubilizing agents are described herein, below. In one example, the solubilizing agent has a structure according to Formula (III) described herein below. In another example, the solubilizing agent has a structure according to Formula (IV), wherein the integer a is selected from 0 and 1:

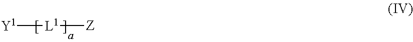

In Formula (IV), Z is a hydrophobic moiety. In one example, Z is a member selected from sterols (e.g., cholesterol or sitosterol), tocopherols (e.g., alpha-tocopherol), tocotrienol and ubiquinols (e.g., ubiquinol-50) and derivatives or homologues thereof. A person of skill in the art will know that the hydrophobic moiety (e.g., tocopherol) when linked to Y¹ is an analog of the molecule, wherein a hydrogen atom is replaced with the moiety “Y¹-[L¹]ₐ-”.

In Formula (IV), Y¹ is a linear or branched hydrophilic moiety including at least one polymeric moiety, wherein each polymeric moiety is a member independently selected from poly(alkylene oxides) (e.g., PEG) and polyalcohols. Exemplary lipophilic moieties are described herein, below, each of which is useful in this embodiment. In one example, the lipophilic moiety is poly(ethylene glycol) (PEG) or methylated PEG (mPEG).

In Formula (IV), L¹ is a linker moiety that covalently links the hydrophobic moiety Z and the hydrophilic moiety Y¹. Exemplary linker moieties are described herein below. In one example, L¹ is selected from a single bond, substituted or unsubstituted alkyl, substituted or unsubstituted heteroalkyl, substituted or unsubstituted aryl, substituted or unsubstituted heteroaryl and substituted or unsubstituted heterocycloalkyl. In one embodiment, L¹ includes a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkyl chain, optionally incorporating at least one functional group. Exemplary functional groups according to this embodiment include ether, thioether, ester, carbonamide, sulfonamide, carbonate and urea groups. In a particular example, the solubilizing agent is selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In an exemplary embodiment, the ratio of the lipophilic bioactive molecule to the solubilizing agent is from about 1:0.3 (w/w) to about 1:20 (w/w). In an exemplary embodiment, the ratio of the lipophilic bioactive molecule to said solubilizing agent is from about 1:1 (w/w) to about 1:20 (w/w). In another exemplary embodiment, the ratio of the lipophilic bioactive molecule to said solubilizing agent is from about 1:1 (w/w) to about 1:10 (w/w). In another exemplary embodiment, the ratio of the lipophilic bioactive molecule to said solubilizing agent is from about 1:1.3 (w/w) to about 1:5 (w/w). In another exemplary embodiment, the ratio of the lipophilic bioactive molecule to said solubilizing agent is from about 1:2 (w/w) to about 1:4 (w/w). In another exemplary embodiment, the ratio of the lipophilic bioactive molecule to said solubilizing agent is about 1:3 (w/w). In an exemplary embodiment, the ratio of the lipophilic bioactive molecule to said solubilizing agent is from about 1:0.3 (w/w) to about 1:1 (w/w). In an exemplary embodiment, the ratio of the lipophilic bioactive molecule to said solubilizing agent is from about 1:0.5 (w/w) to about 1:2 (w/w).

### Water-Soluble Reducing Agent

In an exemplary embodiment, the water-soluble reducing agent contained in the formulation (e.g., aqueous formulation) protects the lipophilic bioactive molecule from chemical degradation (e.g., oxidative and/or light-induced processes). For example, addition of vitamin C or a water-soluble vitamin C derivative to a formulation containing DHA and PTS will serve to prolong the chemical stability of DHA in the aqueous formulation for at least several weeks. In other embodiments, the water-soluble reducing agent is added to the formulation in an amount sufficient to both reduce and stabilize the lipophilic bioactive molecule after reduction. For example, ubiquinone and a solution of a solubilizing agent in water (e.g., PTS) are mixed. Upon mixing of the components, micelles of a small particle size are formed (e.g., average particle size between about 20 and about 30 nm). A water-soluble reducing agent, such as vitamin C or a vitamin C derivative, is then added. The water-soluble reducing agent reduces the ubiquinone to ubiquinol. Excess of water-soluble reducing agent serves to protect against ubiquinol degradation (e.g., oxidation to ubiquinone).

In this function, the water-soluble reducing agent can be considered a stabilizer. In one example, the reducing agent is added in an over-stoichiometric mol ratio with respect to the lipophilic bioactive molecule. In another embodiment, the ratio of lipophilic bioactive molecule to water-soluble reducing agent in the formulation is between about 100:1 and about 1:20 (w/w). In yet another embodiment, the ratio of lipophilic bioactive molecule to water-soluble reducing agent in the formulation is between about 50:1 and about 1:10 (w/w). In yet another embodiment, the ratio of lipophilic bioactive molecule to water-soluble reducing agent in the formulation is between about 20:1 and about 1:10 (w/w). In yet another embodiment, the ratio of lipophilic bioactive molecule to water-soluble reducing agent in the formulation is between about 10:1 and about 1:10 (w/w). In yet another embodiment, the ratio of lipophilic bioactive molecule to water-soluble reducing agent in the formulation is between about 1:1 (w/w) and about 1:10 (w/w), between about 1:1 and about 1:8 (w/w), about 1:1 and about 1:6 (w/w) or between about 1:1 and about 1:4 (w/w). In yet another embodiment, the ratio of lipophilic bioactive molecule to water-soluble reducing agent in the formulation is between about 1:1 and about 1:3 (w/w). In yet another embodiment, the ratio of lipophilic bioactive molecule to water-soluble reducing agent in the formulation is between about 1:1 and about 1:2 (w/w). A person of skill in the art will understand that at least part of the reducing agent can be present in its “oxidized” form. For example, when vitamin C is used as the water-soluble reducing agent, at least part of the vitamin C can be present in the formulation as dehydroascorbic acid.

In one example, in which the lipophilic bioactive molecule is an omeg-fatty acid (e.g., omega-3-, omega-6- or omega-9-fatty acid), the ratio of fatty acid to water-soluble reducing agent in the formulation is between about 100:1 and about 10:1 (w/w).

In another example, in which the lipophilic bioactive molecule is a carotenoid (e.g., lutein, astaxanthin, canthaxanthin, fucoxanthin or lycopene), the ratio of carotenoid to water-soluble reducing agent in the formulation is between about 10:1 and about 1:10 (w/w).

In one example according to any of the above embodiments, the lipophilic bioactive molecule in the formulation is essentially stable to chemical degradation (e.g., oxidation). In one example, the formulation is essentially stable for at least 30, 60, 90, 120, 160 or 180 days when stored at a temperature below about 25° C. (e.g., about 4° C. or about 10° C.). Typically, the formulations are stored at about 4° C. At this temperature, the formulations are typically stable for at least 4, 5 or 6 month.

In one example, according to any of the above embodiments the formulation is contained in a soft-gelatin capsule. A person of skill will understand that formulations suitable for incorporation into soft-gelatin capsules typically contain less than about 5%, preferably less than about 4%, more preferably less than about 3% and most preferably less than about 2% (w/w) of water. Hence, in one example, the formulation includes less than 5% (w/w) of water.

The lipophilic bioactive molecule in the above formulations can be any lipophilic bioactive molecule, such as those described herein. Exemplary lipophilic bioactive molecules according to any of the above embodiments include those molecules that are difficult to stabilize using known methods. In one example, according to any of the above embodiments, the lipophilic bioactive molecule is a member selected from omega-3-fatty acids (e.g., docosahexaenoic acid (DHA), eicosapentaenoic acid (EPA) and alpha-linolenic acid (ALA)), omega-6-fatty acids, omega-9-fatty acids, carotenoids, essential oils, flavor oils and lipophilic vitamins. Exemplary carotenoids include lutein, astaxanthin, lycopene, fucoxanthin and canthaxanthin. Additional carotenoids (e.g., xanthophylls) are described herein, below.

In one example, according to any of the above embodiments, the formulation is an aqueous formulation and includes at least about 5% (w/w) of water. In other examples, the aqueous formulation includes at least about 10%, at least about 20%, at least about 30% at least about 40% or at least about 50% (w/w) of water. In another example, the aqueous formulation includes more than 50% (w/w) of water. For example, the aqueous formulation includes at least about 55%, at least about 60%, at least about 65%, at least about 70%, at least about 75% or at least about 80% (w/w) of water. In a further example, the aqueous formulation includes more than 80% (w/w) water. For example, the aqueous formulation includes at least about 85%, at least about 90%, at least about 92%, at least about 94% or at least about 96% (w/w) of water.

In one example, the lipophilic bioactive molecule is solubilized in the aqueous formulation through the formation of micelles that are formed between the lipophilic bioactive molecule and the solubilizing agent. The particle size of the formed micelles in solution may be measured using a dynamic light scattering (DLS) detector. Typically, smaller particle sizes are associated with a greater tendency of the human body to absorb active ingredients contained in micelles. In one example, the small size of the micelles, enhances or improves the taste or smell of a flavoring agent. In one embodiment, the aqueous formulations of the invention include micelles with particle sizes smaller than the particle sizes produced by known formulations.

In one embodiment, the aqueous formulation of the invention is essentially clear (e.g., free of visible precipitation, cloudiness or haziness). In one example, the lipophilic bioactive molecule of the invention is formulated with PTS resulting in an aqueous formulation that is essentially clear. Clear formulations of the invention can be colored. In one example, the formulation is essentially clear when the micelles have a particle size below the visible size (e.g., below 100 nm). Hence, in another exemplary embodiment, the micelles formed between the lipophilic bioactive molecule and the solubilizing agent, have a median (average) particle size of less than about 100 nm. In another example, the micelles formed between the lipophilic bioactive molecule and the solubilizing agent, have a median particle size of less than about 90 nm, less than about 80 nm, less than about 70 nm or less than about 60 nm. In a further example, the micelles formed between the lipophilic bioactive molecule and the solubilizing agent, have a median particle size of less than about 50 nm, less than about 40 nm or less than about 30 nm. In another exemplary embodiment, the average particle size is from about 10 nm to about 90 nm. Another exemplary average particle size is from about 5 nm to about 70 nm, preferably from about 10 nm to about 50 nm, more preferably from about 10 nm to about 30 nm. In a particular example, the micelles formed between the lipophilic bioactive molecule and the solubilizing agent, have a median particle size between about 30 nm and about 20 nm (e.g., about 25 nm).

In another example, the aqueous formulation does not include an alcoholic solvent. For example, the presence of an alcoholic solvent can disrupt the proper formation of the emulsion and can destroy already formed micelles. Exemplary alcoholic solvents that can be detrimental to the micelles formed in aqueous formulations include solvents, such as ethanol, methanol, propanol, butanol and higher alcohols (e.g., C₅-C₂₀ alcohols). Alcoholic solvents also include polyhydric alcohols, such as ethylene glycol, propylene glycol, glycerol and the like. The term “alcoholic solvent” does not include polymers, such as polymeric versions of the above listed polyhydric alcohols (e.g., poly(alkylene oxides)), such as PEG or PPG).

In one example, according to any of the above embodiments, the concentration of lipophilic bioactive molecule in the formulation is at least about 20 mg/mL and can be as high as about 60, about 80, about 100 or more than about 100 mg/mL. In one example, the concentration of lipophilic bioactive molecule in the aqueous formulation of the invention is at least about 1 mg/mL. In another example, the concentration of lipophilic bioactive molecule in the aqueous formulation is at least about 5 mg/mL or at least about 10 mg/mL. In yet another example, the concentration of lipophilic bioactive molecule in the aqueous formulation is at least about 20 mg/mL, at least about 30 mg/mL, at least about 40 mg/mL, at least about 50 mg/mL, at least about 60 mg/mL, at least about 70 mg/mL or at least about 80 mg/mL. In a further example, the concentration of lipophilic bioactive molecule in the aqueous formulation is at least about 85 mg/mL, at least about 90 mg/mL, at least about 95 mg/mL or at least about 100 mg/mL. In yet another example, the concentration of lipophilic bioactive molecule in the aqueous formulation is at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL or at least about 200 mg/mL. In another example, the concentration of lipophilic bioactive molecule in the aqueous formulation is greater than 200 mg/mL

In one example, according to any of the above embodiments, the lipophilic bioactive molecule is ubiquinol (e.g., ubiquinol-50) (ubiquinol formulation). Hence, in one embodiment, the invention provides a water-soluble formulation including ubiquinol, a water-soluble reducing agent and a solubilizing agent of the invention. Exemplary solubilizing agents are described herein, below. In one example, the solubilizing agent has a structure according to Formula (IV) described herein.

In one example, the ubiquinol formulation further includes ubiquinone (e.g., CoQ₁₀). In another example, the water-soluble reducing agent used in the ubiquinol formulations is capable of reducing ubiquinone (e.g., CoQ₁₀) to its corresponding ubiquinol (e.g., ubiquinol-50). For example, the formulation is formed by reducing ubiquinone to ubiquinol in situ using a water-soluble reducing agent of the invention (e.g., vitamin C). Such methods are described herein. In one example, this reaction is essentially quantitative. Hence, in another example, the ubiquinol formulation is essentially free of ubiquinone (e.g., CoQ₁₀). Formulations including a small ubiquinone:ubiquinol ratio (e.g., below about 10%) are generally preferred because the reduced version of the molecule is considered the bioactive form and is also more bioavailable than the corresponding ubiquinone. In one example, the ratio of ubiquinone to ubiquinol is less than about 50%, less than about 40%, less than about 30%, less than about 20% or less than about 10% (w/w). In a particular example, the ratio of ubiquinone to ubiquinol in the ubiquinol formulation is less than about 8%, less than about 6%, less than about 4% or less than about 2% (w/w). In another example, the ratio of ubiquinone to ubiquinol in the ubiquinol formulation is less than about 1.8%, less than about 1.6%, less than about 1.4%, less than about 1.2%, or less than about 1% (w/w). In a further example, the ubiquinol formulation is essentially free of ubiquinone (e.g., below HPLC-detectable level). In one example, the ratio of ubiquinol to corresponding ubiquinone is at least about 95%. In another example, the ratio of ubiquinol to ubiquinone is at least about 20, about 40, about 60 or about 80% (w/w).

In a further example according to any of the above embodiments, the ubiquinol formulation contains an amount of the water-soluble reducing agent, which is sufficient to diminish or prevent the chemical degradation of the ubiquinol (e.g., oxidation or re-oxidation to ubiquinone) over time. In this function, the water-soluble reducing agent can be considered a stabilizer. In one example, the reducing agent is added in an over-stoichiometric mol ratio with respect to the ubiquinone/ubiquinol. In another embodiment, the ratio of ubiquinol/ubiquinone to water soluble reducing agent in the ubiquinol formulation is about 1:1 to about 1:50 (w/w). In another embodiment, the ratio of ubiquinol/ubiquinone to water soluble reducing agent in the ubiquinol formulation is about 1:1 to about 1:20 (w/w). In another embodiment, the ratio of ubiquinol/ubiquinone to water soluble reducing agent in the ubiquinol formulation is about 1:1 to about 1:10 (w/w), about 1:1 to about 1:8 (w/w), about 1:1 to about 1:6 (w/w) or about 1:1 to about 1:4 (w/w). In yet another embodiment, the ratio of ubiquinol/ubiquinone to water soluble reducing agent in the ubiquinol formulation is about 1:1 to about 1:3 (w/w). A person of skill in the art will understand that at least part of the reducing agent can be present in its “oxidized” form. For example, when vitamin C is used as the water-soluble reducing agent, at least part of the vitamin C may be present in the ubiquinol formulation as dehydroascorbic acid.

In one example according to any of the above embodiments, the ubiquinol in the ubiquinol formulation is essentially stable to chemical degradation (e.g., oxidation to ubiquinone). In one example, the ubiquinol is essentially stable for at least 30, 60, 90, 120, 160 or 180 days when stored at a temperature below about 25° C. (e.g., about 4° C. or about 10° C.). Typically, ubiquinol formulations are stored at about 4° C. At this temperature, the ubiquinol formulations are stable for at least 90 days. I another embodiment, the aqueous ubiquinol formulation, when stored at about 4° C., is stable for at least 180 days. The extraordinary stability of the reduced form of ubiquinone in the formulations of the current invention constitutes a significant advancement in the art. Such stability is accomplished through a synergistic effect between using an amphiphilic solubilizing agent of the invention, which allows for the formation of unusually small micelles, and the presence of a water-soluble (as opposed to lipid-soluble) reducing agent, such as vitamin C. The discovery that a hydrophobic molecule enclosed in micelles, which expose hydrophilic moieties on their surface, can be effectively reduced by a hydrophilic reducing agent, is surprising.

Another advantage of the above ubiquinol formulations is that they can be essentially colorless. Ubiquinol is much lighter in color (e.g., slight yellow) than the corresponding ubiquinone, which is typically dark orange. The lighter color can be more appealing to the consumer and provides a greater flexibility with respect to the use of coloring agents and other additives. Another advantage of the current formulations stems from the fact that they combine at least two beneficial ingredients (ubiquinone/ubiquinol and vitamin C/vitamin C derivative) in a single preparation. This can provide greater convenience to a consumer. When PTS is used as the solubilizing agent, the instant formulations provide a combination of at least three beneficial ingredients (ubiquinone/ubiquinol, vitamin C/vitamin C derivative and vitamin E) in a single preparation.

In another example according to any of the above embodiments, the ubiquinol formulation is an aqueous formulation. The aqueous formulation can be formed by combining ubiquinone (e.g., CoQ₁₀) with a solution of a solubilizing agent in water forming an emulsion, and subsequently contacting the emulsion with a water-soluble reducing agent to reduce the ubiquinone to ubiquinol. Hence, in another example, the ubiquinol is emulsified in the formulation in the form of micelles that include the ubiquinol and the solubilizing agent. In a typical emulsion of the invention, the micelles are surprisingly small in size. In one example, the micelles are between about 20 and about 30 nm. In another example, the small size of the micelles causes the emulsion to be essentially clear in appearance even at high compound concentrations (e.g., 40, 60, 80 or 100 mg/mL). In one example, the ubiquinol concentration in the aqueous formulations of the invention is at least about 20 mg/mL and can be as high as about 60, about 80, about 100 or more than about 100 mg/mL.

In one example, according to any of the above embodiments, the formulation is water-soluble (water-soluble formulation). In one example, the invention provides a mixture of a water-soluble formulation of the invention and a carrier suitable for topical application. For example, the water-soluble formulation of the invention is used in a skin-care product, such as a cream or ointment.

### Beverages

In another example, the invention provides a mixture between a formulation of the invention (e.g., a water-soluble formulation) and an original beverage to create a beverage of the invention. The original beverage can be any beverage (e.g., a clear beverage). Exemplary original beverages are described herein and include carbonated or non-carbonated waters, flavored waters, soft drinks and the like. In one example, the mixture (beverage of the invention) includes between about 1 mg/L and about 1000 mg/L of solubilized lipophilic bioactive molecule. In another example, the mixture includes between about 10 mg/L and about 500 mg/L of solubilized lipophilic bioactive molecule. In yet another example, the mixture includes between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized lipophilic bioactive molecule. In a further example, the mixture includes between about 20 mg/L and about 250 mg/L, between about 20 mg/L and about 200 mg/mL, between about 20 mg/L and about 150 mg/mL, between about 20 mg/L and about 100 mg/mL, or between about 20 mg/L and about 80 mg/mL, between about 20 mg/L and about 60 mg/mL, between about 20 mg/L and about 40 mg/mL of solubilized lipophilic bioactive molecule.

In a particular example according to any of the above embodiments, the invention provides a mixture between a ubiquinol formulation of the invention (e.g., an aqueous ubiquinol formulation) and an original beverage (e.g., carbonated or non-carbonated water) to form a ubiquinol beverage.

Hence, in another aspect, the invention provides a non-alcoholic beverage comprising (a) solubilized ubiquinol (e.g., ubiquinol-50), (b) a water-soluble reducing agent of the invention (e.g., vitamin C) and (c) a solubilizing agent of the invention.

In an exemplary embodiment, the ubiquinol beverage contains between about 1 mg/L and about 1000 mg/L of solubilized ubiquinol. In another example, the beverage contains between about 10 mg/L and about 500 mg/L of solubilized ubiquinol. In yet another example, the mixture includes between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized ubiquinol. In a further example, the mixture includes between about 20 mg/L and about 250 mg/L, between about 20 mg/L and about 200 mg/mL, between about 20 mg/L and about 150 mg/mL, between about 20 mg/L and about 100 mg/mL, or between about 20 mg/L and about 80 mg/mL, between about 20 mg/L and about 60 mg/mL, between about 20 mg/L and about 40 mg/mL of solubilized ubiquinol.

In another aspect, the invention provides a non-alcoholic beverage including (a) solubilized ubiquinone (e.g., CoQ₁₀), (b) a solubilizing agent of the invention, and optionally (c) a water-soluble reducing agent of the invention (e.g., vitamin C).

In one exemplary embodiment, the ubiquinone beverage contains between about 1 mg/L and about 1000 mg/L of solubilized ubiquinone. In another example, the beverage contains between about 10 mg/L and about 500 mg/L of solubilized ubiquinone. In yet another example, the beverage includes between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized ubiquinone. In a further example, the beverage includes between about 20 mg/L and about 250 mg/L, between about 20 mg/L and about 200 mg/mL, between about 20 mg/L and about 150 mg/mL, between about 20 mg/L and about 100 mg/mL, between about 30 and about 100 mg/mL, between about 20 mg/L and about 80 mg/mL, between about 20 mg/L and about 60 mg/mL, or between about 20 mg/L and about 40 mg/mL of solubilized ubiquinone.

In one example according to any of the above aspects, the solubilizing agent has a structure according to Formula (III) described herein below. In another example, the solubilizing agent has a structure according to Formula (IV):

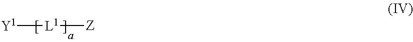

wherein the integer a, Y¹, L¹ and Z are defined as herein above. In another example, the solubilizing agent is selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In one example according to any of the above embodiments, the beverage includes ubiquinol and further includes ubiquinone. In another example according to any of the above embodiments, the beverage includes ubiquinol (e.g., ubiquinol-50) but is essentially free of ubiquinone (e.g., CoQ₁₀).

In a further example according to any of the above embodiments, the beverage further includes a coloring agent and/or a flavoring agent. If required, it is possible to add one or more fruit and/or vegetable juice concentrates and/or flavor improvers to the beverage. For example, a mixture of about LIMETTE citrus (e.g., about 1.38 g/l), cassis (e.g., about 1.04 g/l), mango (e.g., about 1.04 g/l) or combinations thereof, can be added to the beverage. In another example, maltodextrin (e.g., about 20 g/l), fructose (e.g., about 50 g/l) or combinations thereof can be added to the beverage. In another example, the finished beverage is subjected to a primary and, optionally, a secondary filtration. In one example, filters with a pore size of about 0.1μ to about 1.5μ can be used.

In a further example according to any of the above embodiments, the ubiquinol beverage includes sufficient water-soluble reducing agent (e.g., vitamin C) to prevent oxidation of ubiquinol to ubiquinone. In another embodiment, the ratio of ubiquinol/ubiquinone to water soluble reducing agent in the beverage is about 1:1 to about 1:10 (w/w). In another embodiment, the ratio of ubiquinol/ubiquinone to water soluble reducing agent in the beverage is about 1:1 to about 1:8 (w/w), about 1:1 to about 1:6 (w/w) or about 1:1 to about 1:4 (w/w). In yet another embodiment, the ratio of ubiquinol/ubiquinone to water soluble reducing agent in the beverage is about 1:1 to about 1:3 (w/w). A person of skill in the art will understand that at least part of the reducing agent can be present in its “oxidized” form. For example, when vitamin C is used as the water-soluble reducing agent, at least part of the vitamin C can be present in the beverage as dehydroascorbic acid.

In yet another example according to any of the above embodiments, the ubiquinol or ubiquinone is stably solubilized in the beverage. For example, the beverage is essentially free of ubiquinol precipitation and/or ubiquinone precipitation. Hence, in another example, the beverage is essentially clear. Clarity of a beverage can be assessed using turbidity measurements. In one example, the turbidity of the ubiquinol beverage or ubiquinone beverage is comparable (e.g., not more than 5×) of the turbidity of the control beverage. A suitable control is provided by the corresponding original beverage without solubilized ubiquinol/ubiquinone. The control can optionally include the solubilizing agent. In one example, the turbidity of the ubiquinol/ubiquinone beverage is not more than about 500%, not more than about 400%, not more than about 300% or not more than about 200% higher than the turbidity of the control. In yet another example, the turbidity is not more than about 180%, not more than about 160%, not more than about 140%, not more than about 120% or not more than about 100% higher than the turbidity of the control. The turbidity is 100% higher than the control, when the tubidity of the beverage is twice as high as the turbidity of the control. In a further example, the turbidity of the ubiquinol/ubiquinone beverage is not more than about 80%, not more than about 60%, not more than about 40%, not more than about 20% or not more than about 10% higher than the turbidity of the control.

In another example, the turbidity of the ubiquinol/ubiquinone beverage is stable over time. For example, the turbidity of the beverage is stable over a period of at least 60 days when the beverage is stored at ambient temperature (e.g., below about 25° C.).

After production, the beverage can be packaged into opaque containers which are, in particular, opaque to light, such as visible light and near and far ultraviolet light. It is also possible to use for this purpose containers, for example, cans which cover the entire spectrum of light. Cans made of aluminum or aluminum alloys are preferably used. It is also possible to accommodate the beverage according to the invention in metal foil or aluminum foil sachets. In another example, the beverage is packaged in Tetrapak containers. If the material itself does not have the required property of opacity, it can be coated. There is also the possibility of using an opaque outer pack. In one example, the entire production and filling process takes place with essentially exclusion of light.

In addition, the beverage can be vitaminized. In one example, the beverage includes at least one B vitamin. Exemplary B-vitamins include vitamin B1, vitamin B2, vitamin B3 and vitamin B6 and vitamin B12. In another example, the beverage includes vitamin E. In one example, the vitamin is first formulated into an aqueous composition, which is subsequently added to the beverage. The solubilizing agent used to solubilize the vitamin can be the same solubilizing agent used to solubilize the lipophilic bioactive molecule.

### III. (a) Lipophilic Bioactive Molecule

The lipophilic bioactive molecule of the current invention can be any molecule. In one example, the lipophilic bioactive molecule is selected from compounds with a water-solubility that can be increased using a solubilizing agent of the invention. In another example, the bioactive lipophilic molecule is a molecule associated with pharmaceutical or neutraceutical value. The term “lipophilic bioactive molecule” includes derivatives of such molecules (e.g., esters or amides thereof) and combinations thereof. For example, the lipophilic bioactive molecule has at least one free OH or COOH group, which can be converted to an ester group. In another example, the lipophilic bioactive molecule has at least one free primary or secondary amino group, which can be converted to an amide.

### Oils, Fats and Fatty Acids

In an exemplary embodiment, the lipophilic bioactive molecule is an oil or an oil component. The term “oil” includes oils derived from plant material, such as seed oils, essential oils, oils derived from animals, such as fish or marine oils (e.g., salmon oil) and other fats. In one example, the oil has food grade. Exemplary oils derived from plant materials include flaxseed oil, borage seed oil, garlic oil, pumpkin seed oil, evening primrose oil, wheat germ oil, saw palmetto berry oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, jojoba oil and shea butter. Exemplary essential oils include citrus oils, bergamot oil, jasmine oil, ylang ylang oil, rosemary oil, cinnamon oil, lavender oil, rose oil, rose geranium oil, patchouli oil, neroli oil, vetiver oil and the like. The term essential oil also includes fragrances and flavoring oils (e.g., fruit flavor oils, citrus flavor, almond flavor). Exemplary oils derived from animals include animal fats, such as tallow (e.g., beef tallow), butter, chicken fat, lard, dairy butterfat, or combinations thereof.

In another exemplary embodiment, the lipophilic bioactive molecule is selected from an oil comprising at least one fatty acid (e.g., an essential fatty acid). In another exemplary embodiment, the lipophilic bioactive molecule is selected from an oil comprising at least one type of an omega-3 fatty acid, an oil comprising at least one type of an omega-6 fatty acid, an oil comprising at least one type of an omega-9 fatty acid and an oil comprising at least one type of an omega-12 fatty acid. Exemplary types of omega-3 fatty acids, omega-6 fatty acids, omega-9 fatty acids and omega-12 fatty acid are disclosed herein below in Table 1.

In an exemplary embodiment, the lipophilic bioactive molecule is a member selected from an omega-3 fatty acid, an omega-6 fatty acid, an omega-9 fatty acid, and an omega-12 fatty acid. In an exemplary embodiment, the lipophilic bioactive molecule is an essential fatty acid (EFA), such as a linolenic acid.

In another exemplary embodiment, the lipophilic bioactive molecule is an omega-3 unsaturated fatty acid, such as alpha-linolenic acid (ALA), docosahexaenoic acid (DHA), eicosapentaenoic acid (EPA), stearidonic acid, eicosatetraenoic acid and docosapentaenoic acid. In another exemplary embodiment, the lipophilic bioactive molecule is an omega-6 unsaturated fatty acid, such as linoleic acid, gamma-linolenic acid and arachidonic acid. In yet another exemplary embodiment, the lipophilic bioactive molecule is an omega-9 unsaturated fatty acid, such as oleic acid, eicosenoic acid and erucic acid, as well as conjugated linoleic acid (CLA). In a further exemplary embodiment, the lipophilic bioactive molecule is an omega-12 unsaturated fatty acid. The term “fatty acid” also includes any derivative of those compounds, such as mixed triglycerides, diglyceride esters and alkyl esters, such as methyl- and ethyl esters. Additional fatty acids of the invention are summarized in Table 1, below.

TABLE 1
Exemplary Omega-3, Omega-6 and Omega-9 Fatty Acids

| | | |
|---|---|---|
| Common Name | Lipid Name | Chemical Name |

Omega-3 Fatty Acids

| | | |
|---|---|---|
| α-Linolenic acid (ALA) | 18:3 (n-3) | octadeca-9,12,15-trienoic acid |
| Stearidonic acid | 18:4 (n-3) | octadeca-6,9,12,15-tetraenoic acid |
| Eicosatetraenoic acid | 20:4 (n-3) | eicosa-8,11,14,17-tetraenoic acid |
| Eicosapentaenoic acid (EPA) | 20:5 (n-3) | eicosa-5,8,11,14,17-pentaenoic acid |
| Docosapentaenoic acid | 22:5 (n-3) | docosa-7,10,13,16,19-pentaenoic acid |
| Docosahexaenoic acid (DHA) | 22:6 (n-3) | docosa-4,7,10,13,16,19-hexaenoic acid |

Omega-6 Fatty Acids

| | | |
|---|---|---|
| Linoleic acid | 18:2 (n-6) | 9,12-octadecadienoic acid |
| Gamma-linolenic acid | 18:3 (n-6) | 6,9,12-octadecatrienoic acid |
| Eicosadienoic acid | 20:2 (n-6) | 11,14-eicosadienoic acid |
| Dihomo-gamma-linolenic acid | 20:3 (n-6) | 8,11,14-eicosatrienoic acid |
| Arachidonic acid | 20:4 (n-6) | 5,8,11,14-eicosatetraenoic acid |
| Docosadienoic acid | 22:2 (n-6) | 13,16-docosadienoic acid |
| Adrenic acid | 22:4 (n-6) | 7,10,13,16-docosatetraenoic acid |
| Docosapentaenoic acid | 22:5 (n-6) | 4,7,10,13,16-docosapentaenoic acid |

Omega-9 Fatty Acids

| | | |
|---|---|---|
| oleic acid | 18:1 (n-9) | 9-octadecenoic acid |
| eicosenoic acid | 20:1 (n-9) | 11-eicosenoic acid |
| mead acid | 20:3 (n-9) | 5,8,11-eicosatrienoic acid |
| erucic acid | 22:1 (n-9) | 13-docosenoic acid |
| nervonic acid | 24:1 (n-9) | 15-tetracosenoic acid |

In another exemplary embodiment, the lipophilic bioactive molecule is a botanical extract or a component thereof. Exemplary extracts include extracts of ginseng, hawthorn, St. John's wort, valerian, black cohosh, yohimbe, ephedra, red clover, cayenne, echinacea, arnica (e.g., arnica montana), grape seeds, kava kava, bilberry, gingko biloba, green tea, wine leaf, Japanese knotwood and any other botanical extract available as a dietary supplement.

In an exemplary embodiment, the lipophilic bioactive molecule is a carotenoid, such as carotenes and xanthophylls. In one example, the carotene is a member selected from alpha carotene, beta-carotene and lycopene. In another example, the xanthophyll is a member selected from lutein, astaxanthin, zeaxanthin, cryptoxanthin, canthaxanthin, violaxanthin and fucoxanthin.

In another exemplary embodiment, the lipophilic bioactive molecule is a triterpenoid. Triterpenoids include pentacyclic triterpenoids. In one example, the triterpenoid is a member selected from asiatic acid and ursolic acid. In a further exemplary embodiment, the lipophilic bioactive molecule is a sterol or phytosterol. In one example, the phytosterol is a member selected from β-sitosterol and ergosterol. In another exemplary embodiment, the lipophilic bioactive molecule is a stilbenoid. In one example, the stilbenoid is a member selected from resveratrol and pinosylvin.

In an exemplary embodiment, the lipophilic bioactive molecule is a lipophilic vitamin. In an exemplary embodiment, the vitamin is a member selected from vitamin E and vitamin E derivatives. In an exemplary embodiment, the lipophilic bioactive molecule is a member selected from tocopherols and tocotrienols. In another exemplary embodiment, the lipophilic bioactive molecule is a member selected from alpha-tocopherol and alpha-tocotrienol. In another embodiment, the vitamin is a B-vitamin, such as vitamin B pentapalmitate, vitamin B-6 and vitamin B-12.

In yet another exemplary embodiment, the lipophilic bioactive molecule is a member selected from glutathione, catechins, curcumins, lycopene, lecithin, amino acids (e.g., essential amino acids), L-camitine (or acetyl derivative), alpha-lipoic acid, hyaluronic acid, phytosterols, melatonin and idebenone. In yet another exemplary embodiment, the lipophilic bioactive molecule is a pharmaceutical drug, such as amphotericin B, nystatin, erythromycin, paclitaxel and other anti-tumor agents.

In an exemplary embodiment, the formulation of the invention includes from about 0.01% (w/w) to about 50% (w/w) of a lipophilic bioactive molecule. Formulations including ubiquinol-50, CoQ₁₀ and oils (e.g., DHA oils), typically contain high concentrations of these molecules (e.g., at least 20 mg/mL) as described herein. Formulations including carotenoids (e.g., astaxanthin, fucoxanthin) typically have a lower concentration of these molecules, e.g., due to the fact that they are available only as mixtures (e.g., with oils). A typical carotenoid concentration in the formulation of the invention is between about 1 to 10 mg/mL.

In one example, the formulation includes from about 0.01% (w/w) to about 0.1% (w/w) of a lipophilic bioactive molecule. In another example, the formulation includes from about 0.01% (w/w) to about 0.5% (w/w) of a lipophilic bioactive molecule. In yet another exemplary embodiment, the invention includes from about 0.01% (w/w) to about 1% (w/w) of a lipophilic bioactive molecule. In another exemplary embodiment, the invention includes from about 0.05% (w/w) to about 0.25% (w/w) of a lipophilic bioactive molecule. In a further exemplary embodiment, the invention includes from about 0.1% (w/w) to about 1% (w/w) of a lipophilic bioactive molecule. In another exemplary embodiment, the invention includes from about 0.1% (w/w) to about 0.75% (w/w) of a lipophilic bioactive molecule. In another exemplary embodiment, the formulation includes from about 1% (w/w) to about 3% (w/w) of a lipophilic bioactive molecule. In another exemplary embodiment, the formulation includes from about 1% (w/w) to about 10% (w/w) of a lipophilic bioactive molecule. In another exemplary embodiment, the formulation includes from about 1% (w/w) to about 20% (w/w) of a lipophilic bioactive molecule. In another exemplary embodiment, the formulation includes from about 1% (w/w) to about 30% (w/w) of a lipophilic bioactive molecule. In another exemplary embodiment, the formulation includes from about 1% (w/w) to about 40% (w/w) of a lipophilic bioactive molecule. In another exemplary embodiment, the compositions of the invention contain from about 5% to about 50% by weight of a lipophilic bioactive molecule. In an exemplary embodiment, the composition contains from about 10% to about 30% (w/w) lipophilic bioactive molecule, for example, from about 15% to about 25% (w/w).

### Ubiquinones and Ubiquinols

In an exemplary embodiment, the lipophilic bioactive molecule is an ubiquinone or a reduced form thereof. The reduced form of ubiquinone is generally referred to as an ubiquinol. In an exemplary embodiment, the ubiquinone is ubiquinone Q₁₀ also referred to as coenzyme Q₁₀ (CoQ₁₀). In another exemplary embodiment, the lipophilic bioactive molecule is reduced CoQ₁₀ (ubiquinol-50).

In one embodiment, the ubiquinone/ubiquinol of the current invention has a structure according to Formula (I) or Formula (II):

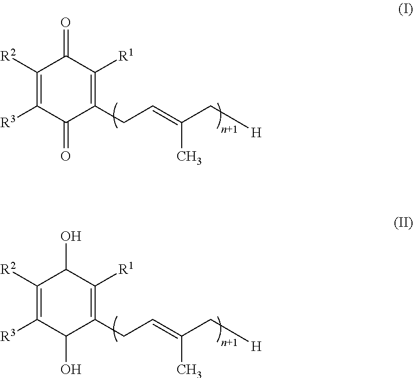

In Formula (I) and Formula (II), the integer n is selected from 1 to 13. R¹, R² and R³ are members independently selected from H, substituted or unsubstituted alkyl and substituted or unsubstituted alkoxy. R² and R³, together with the carbon atoms to which they are attached, are optionally joined to form a 5- to 7-membered ring. In one embodiment, n is 9. In another embodiment, R¹ is methyl. In yet another embodiment, R¹ is methyl and R² and R³ are both methoxy. In a preferred embodiment, the ubiquinone of the invention is CoQ₁₀. A preferred ubiquinol is ubiquinol-50, or reduced CoQ₁₀. Also within the scope of the current invention are compositions including both ubiquinone and ubiquinol.

In one example, the compositions of the invention contain from about 5% to about 50% by weight of ubiquinone/ubiquinol. In an exemplary embodiment, the composition contains from about 10% to about 30% (w/w) ubiquinone/ubiquinol, preferably from about 15% to about 25% (w/w). In one embodiment, the soft gelatin capsules of the invention include ubiquinone/ubiquinol from about 1% to about 30% (w/w). In another embodiment the soft gel capsule includes from about 3% to about 20% (w/w), and preferably from about 5% to about 20% of ubiquinone/ubiquinol.

Ubiquinones/ubiquinols can be purchased commercially from sources such as Kaneka (Japan) and Nisshin (Japan). Ubiquinone/ubiquinols can also be synthesized. Exemplary methods are disclosed in U.S. Pat. Nos. 6,545,184 and 6,852,895, U.S. patent application Ser. Nos. 10/992,270; 11/003,544; 11/304,023 and 10/581,566 and U.S. Provisional Patent Application No. 60/804,920, each of which is incorporated herein in its entirety for all purposes.

### III. (b) Solubilizing Agent

In an exemplary embodiment, the solubilizing agent has a structure according to the following formula:

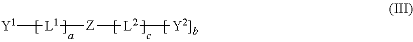

In Formula (III), a, b and c are integers independently selected from 0 and 1. In one example, b is 0. Z is a hydrophobic (lipophilic) moiety. In one example, Z is a sterol (e.g., beta-sitosterol, cholesterol). In another example, Z is a tocopherol (e.g., alpha-tocopherol, alpha-tocotrienol) or a derivative or homologue thereof. In yet another example, Z is a ubiquinol. A person of ordinary skill in the art will understand that the residue of the hydrophobic moiety is the entire hydrophobic molecule, except for at least one hydrogen atom, which is replaced with the hydrophilic moiety or the linker-hydrophilic moiety cassette (e.g., hydrogen atom of esterified hydroxyl group, such as 3-β-hydroxyl group of cholesterol or sitosterol or 6-hydroxyl group of α-tocopherol).

In Formula (III), Y¹ and Y² are linear or branched hydrophilic moieties comprising at least one polymeric moiety, wherein each polymeric moiety is independently selected. In one example, Y¹ and Y² are independently selected from hydrophilic (i.e., water-soluble) polymers. In another example, Y¹ and Y² are members independently selected from poly(alkylene oxides) (i.e., polyethers), polyalcohols, polysaccharides (e.g., polysialic acid), polyamino acids (e.g., polyglutamic acid, polylysine), polyphosphoric acids, polyamines and derivatives thereof. Exemplary poly(alkylene oxides) include polyethylene glycol (PEG) and polypropylene glycol (PPG). PEG derivatives include those, in which the terminal hydroxyl group is replaced with another moiety, such as an alkyl group (e.g., methyl, ethyl or propyl). In one example, the hydrophilic moiety is methyl-PEG (mPEG).

PEG is usually a mixture of oligomers characterized by an average molecular weight. In one example, the PEG has an average molecular weight from about 200 to about 5000. In another examplary embodiment, PEG has an average molecular weight from about 500 to about 1500. In another examplary embodiment, PEG has an average molecular weight from about 500 to about 700 or about 900 to about 1200. In one example, the lipophilic moiety of the solubilizing agent is PEG-400. In one example, the lipophilic moiety of the solubilizing agent is PEG-600. Both linear and branched PEG moieties can be used as the hydrophilic moiety of the solubilizing agent in the practice of the invention. In an exemplary embodiment, PEG has between 1000 and 5000 subunits. In an exemplary embodiment, PEG has between 100 and 500 subunits. In an exemplary embodiment, PEG has between 10 and 50 subunits. In an exemplary embodiment, PEG has between 1 and 25 subunits. In an exemplary embodiment, PEG has between 15 and 25 subunits. PEG has between 5 and 100 subunits. In an exemplary embodiment, PEG has between 1 and 500 subunits.

In a further embodiment the poly(ethylene glycol) is a branched PEG having more than one PEG moiety attached. Examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660 and WO 02/09766; as well as Kodera Y., Bioconjugate Chemistry 5: 283-288 (1994); and Yamasaki et al., Agric. Biol. Chem., 52: 2125-2127, 1998, all of which are incorporated herein by reference in their entirety. Exemplary branched PEG moieties involve a branched core molecule having at least two PEG arms attached, each at a different attachment point.

In an exemplary embodiment, at least one of Y¹ and Y² includes a moiety having the following structure:

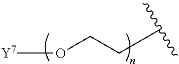

wherein Y⁷ is a member selected CH₃ and H, and n is a member selected from 1 to 5000 (e.g., 1 to 2500). In an exemplary embodiment, n is a member selected from 1000-5000. In an exemplary embodiment, n is a member selected from 1-500. In an exemplary embodiment, n is a member selected from 5-100. In an exemplary embodiment, n is a member selected from 100-500. In an exemplary embodiment, n is a member selected from 10-50. In an exemplary embodiment, n is a member selected from 1-25.

In an exemplary embodiment, Y¹ and/or Y² is a member selected from:

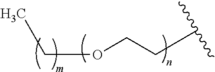

wherein m is a member selected from 0 to 30, and n is a member selected from 1 to 5000. In an exemplary embodiment, m is a member selected from 5-20. In an exemplary embodiment, m is a member selected from 8-15. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In an exemplary embodiment, Y¹ and/or Y² is a member selected from:

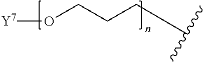

wherein Y⁷ is a member selected CH₃ and H, and n is a member selected from 1 to 2500. In an exemplary embodiment, m is a member selected from 5-20. In an exemplary embodiment, m is a member selected from 8-15. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In an exemplary embodiment, Y¹ and/or Y² is a member selected from:

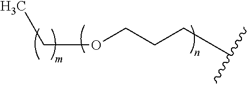

wherein m is a member selected from 0 to 30, and n is a member selected from 1 to 2500. In an exemplary embodiment, m is a member selected from 5-20. In an exemplary embodiment, m is a member selected from 8-15. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In one example, the hydrophilic molecule has a reactive functional group, which can be used to chemically attach the hydrophilic molecule to the hydrophobic moiety (e.g., sterol, tocopherol or ubiquinol), either directly or through a linker moiety. Examples of functional groups include esterifiable hydroxyl groups, carboxy groups and amino groups. In one example, the hydrophilic moiety is a polyether (e.g., polyalkylene glycol). The term “polyalkylene glycol” includes polymers of lower alkylene oxides, in particular polymers of ethylene oxide (polyethylene glycols) and propylene oxide (polypropylene glycols), having an esterifiable hydroxyl group at least at one end of the polymer molecule, as well as derivatives of such polymers having esterifiable carboxylic acid groups. The residue of the hydrophilic moiety is the entire hydrophilic molecule, except for the atom involved in forming the bond to the hydrophobic moiety or the linker moiety (i.e. hydrogen atom of an esterified hydroxyl group).

In Formula (III), L¹ and L² are linker moieties. In one example, L¹ and L² are independently selected from a single bond, substituted or unsubstituted alkyl, substituted or unsubstituted heteroalkyl, substituted or unsubstituted aryl, substituted or unsubstituted heteroaryl and substituted or unsubstituted heterocycloalkyl.

In one example, at least one of L¹ and L² includes a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkyl chain, optionally incorporating at least one functional group. Exemplary functional groups according to this embodiment include ether, thioether, ester, carbonamide, sulfonamide, carbonate and urea groups.

In another example, at least one of L¹ and L² includes a moiety having the following formula:

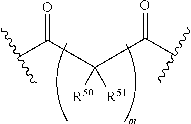

wherein m is an integer selected from 1 to 30. In one example, m is selected from 2 to 20. Each R⁵⁰ and each R⁵¹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

In another example, at least one of L¹ and L² includes a moiety having the following formula:

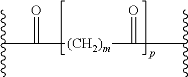

wherein m is an integer selected from 1 to 18 (e.g., from 1 to 10); and p is an integer selected from 0 and 1.

When p is 1, the linker can be derived from an alkanedioic acid of the general formula HOOC—(CH₂)ₘ—COOH. Preferred linkers include diesters derived from an alkanedioic acid. For the practice of the present invention, alkanedioic acids with m from 0 to 18 are preferred, those with m from 6 to 10 being particularly preferred. In some embodiments, sebacic acid (m=8) is particularly preferred. In another exemplary embodiment, the solubilizing agent includes the moiety:

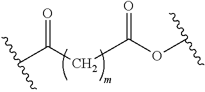

wherein m is a member selected from 4, 6, 8, 10, 12 and 14. In one example, m is 8 and the linker is derived from sebacic acid.

Other preferred linkers include diethers derived from a substituted alkane. In an exemplary embodiment the substituted alkane has the general structure X—(CH₂)ₙ—X′ wherein X and X′ independently represent a leaving group such as a halogen atom or a tosylate group. For the practice of the present invention, substituted alkanes with n from 0 to 18 are preferred, those with n from 6 to 10 being particularly preferred. The ether derived from a 1,10-substituted decane (n=10), such as 1,10-dibromodecane is most particularly preferred.

In yet another example, the solubilizing agent includes a moiety, which is a member selected from:

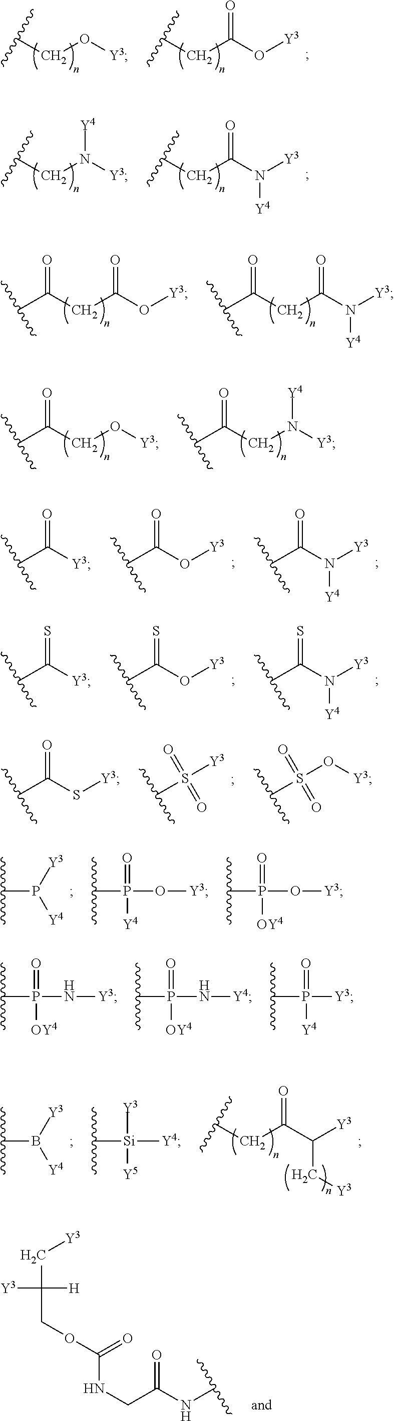

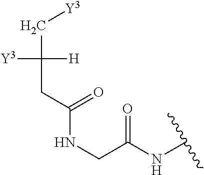

wherein the integer n is a member selected from 0 to 18. Y³ is a member selected from Y¹ and Y². Y⁴ and Y⁵ are members independently selected from H, substituted or unsubstituted alkyl, substituted or unsubstituted heteroalkyl, substituted or unsubstituted aryl, substituted or unsubstituted heteroaryl and substituted or unsubstituted cycloalkyl and substituted or unsubstituted heterocycloalkyl.

In an exemplary embodiment, the solubilizing agent includes a branched linker. In one example, at least one of L¹ and L² includes a moiety having the following formula:

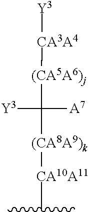

wherein in the integers j and k are independently selected from 0 to 20. A¹, A², A³, A⁴, A⁵, A⁶, A⁷, A⁸, A⁹, A¹⁹ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹²A¹³, —OA¹² and —SiA¹²A¹³. A¹² and A¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In one embodiment the solubilizing agent is not PCS (polyoxyethanyl-cholesteryl sebacate). In another embodiment, the solubilizing agent is not TPGS (polyoxyethanyl-a-tocopheryl succinate).

In one exemplary embodiment, the solubilizing agent has a structure according to one of the following formulae:

Y¹—Z—Y²;

Y¹-L¹-Z—Y²;

Y¹—Z-L²-Y²; and

Y¹-L¹-Z-L²-Y²

wherein a, Y¹, Z and L¹ are defined as herein above. All embodiments described herein above for Formula (III) equally apply to the examples of this paragraph.

In one example, the solubilizing agent has a structure according to Formula (IV), wherein the integer a, Y¹, Z and L¹ are defined as herein above:

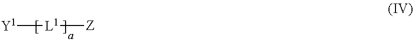

All embodiments described herein above for Formula (III) equally apply to the examples of this paragraph.

### Solubilizing Agents Wherein Z is a Sterol

In an exemplary embodiment, Z is a sterol. In one example, the sterol is a member selected from 7-dehydrocholesterol, campesterol, sitosterol, ergosterol and stigmasterol. Cholesterol and sitosterol are preferred sterols, sitosterol being particularly preferred. In an exemplary embodiment, Z is member selected from a zoosterol and a phytosterol. In another exemplary embodiment, Z is a sterol with an oxygen atom at the 3-position of the A-ring. In an exemplary embodiment, in Formula (IV), Z has a structure according to the following formula:

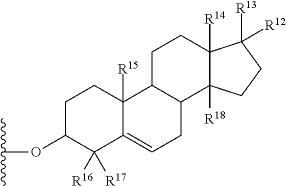

wherein at least one of R¹² and R¹³ is substituted or unsubstituted alkyl. R¹⁴, R¹⁵, R¹⁶, R¹⁷, and R¹⁸ are independently H, or substituted or unsubstituted alkyl. In an exemplary embodiment, Z is a member selected from

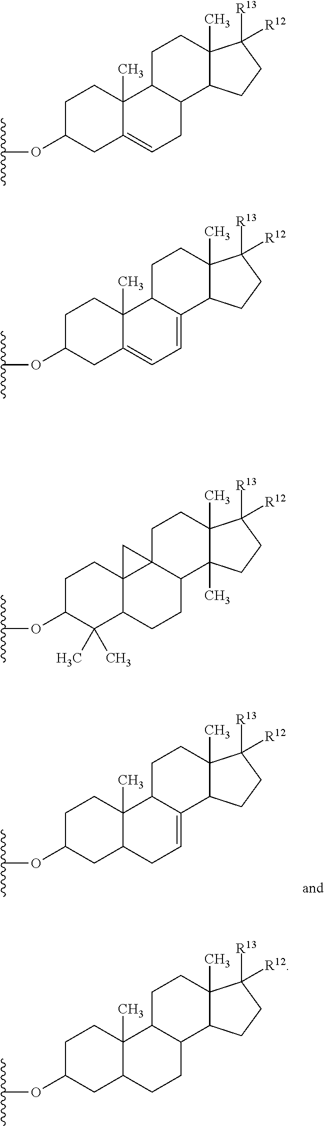

In another example, I the above structures, at least one of R¹² and R¹³ is H. Exemplary sterols according to this embodiment include:

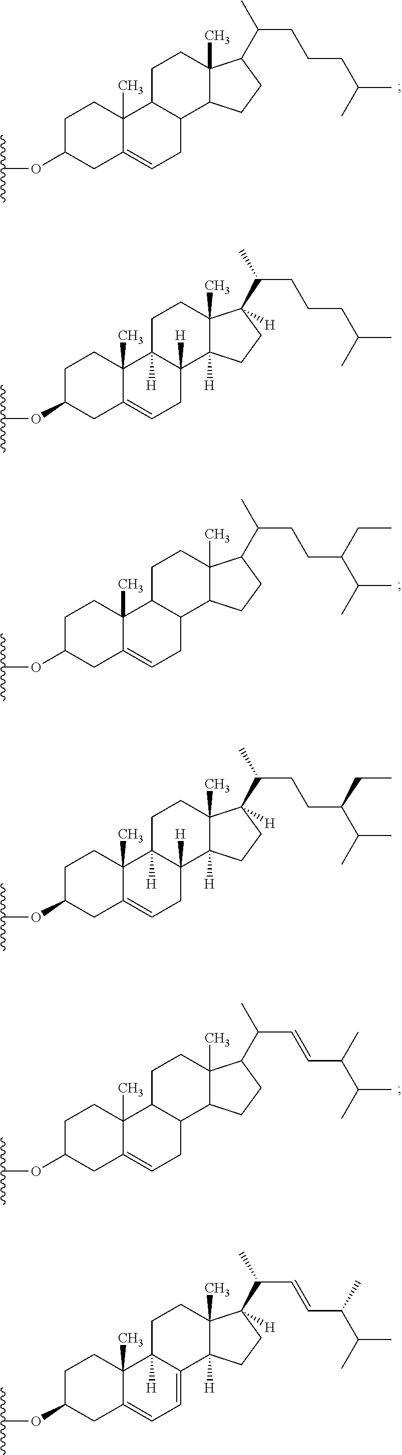

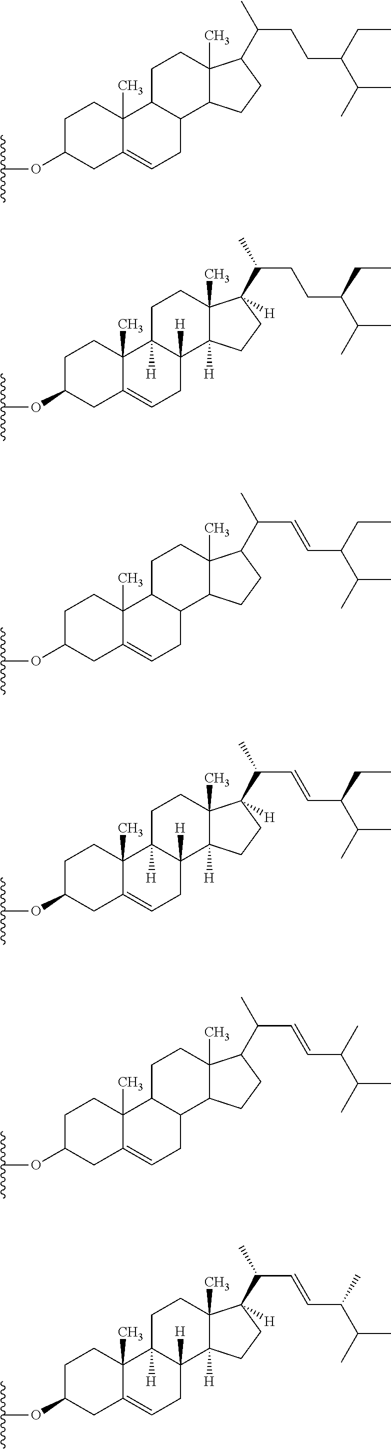

Additional examples of sterols include episterol, cycloartenol, avenasterol, 24-methylenecycloartenol.

### Solubilizing Agents Wherein Z is a Tocopherol or a Tocotrienol

In another embodiment, Z is a member selected from a substituted or unsubstituted tocopherol and a substituted or unsubstituted tocotrienol. In one example, Z is an α-, β-, γ-, or A-tocopherol. α-(+)-Tocopherol and α-(±)-tocopherol are preferred tocopherols, with synthetic DL tocopherol being particularly preferred for PTS. In an exemplary embodiment, Z has a structure according to the following formula:

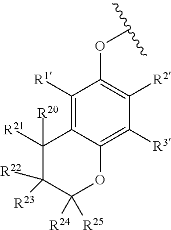

wherein R^{1′}, R^{2′} and R^{3′} are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R^{2′} and R^{3′}, together with the carbon atoms to which they are attached, are optionally joined to form a 5- to 7-membered ring. R²⁰, R²¹, R²², R²³, R²⁴ and R²⁵ are members selected from H, halogen, nitro, cyano, OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. In an exemplary embodiment, at least one of R²⁴ and R²⁵ comprises an isoprene moiety.

In another exemplary embodiment, R^{1′}, R^{2′} and R^{3′} are members independently selected from H and methyl. In another exemplary embodiment, R^{3′} is methyl, R^{2′} is methyl and R^{1′} is methyl. In another exemplary embodiment, R^{3′} is methyl, R^{2′} is H and R^{1′} is methyl. In another exemplary embodiment, R^{3′} is methyl, R^{2′} is methyl and R^{1′} is H. In another exemplary embodiment, R^{3′} is methyl, R^{2′} is H and R^{1′} is H.

In one example, Z has a structure according to the following formulae:

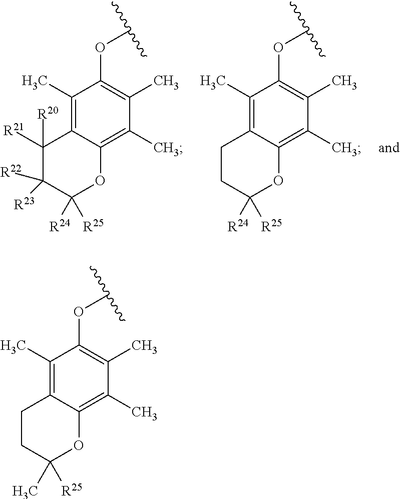

wherein R²⁵ is a member selected from substituted or unsubstituted alkyl and substituted or unsubstituted heteroalkyl. In one example, R²⁴ is methyl. In another example, R²⁵ includes a moiety having a structure selected from the following formulae:

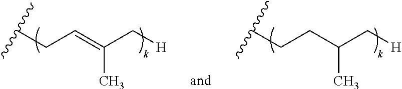

wherein k is an integer selected from 1 to 12. In an exemplary embodiment, k is from 2 to 6. In another exemplary embodiment, k is 3.

In an exemplary embodiment, the solubilizing agent has a structure according to the following formula:

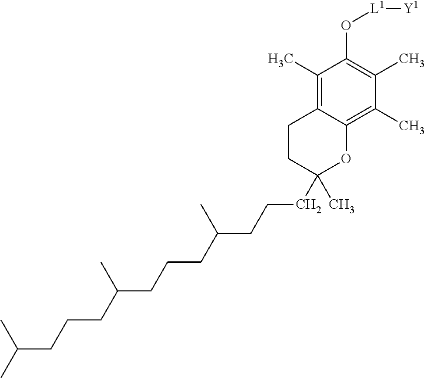

In an exemplary embodiment, the moiety L¹-Y¹ has a structure according to the following formula:

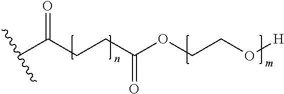

wherein n is member selected from 1 to 20, m is a member selected from 1 to 5000. In another exemplary embodiment, n is 4. In another exemplary embodiment, m is a member selected from 1 to 2,500.

### Solubilizing Agents Wherein Z is Ubiquinol

In an exemplary embodiment, Z is an ubiquinol. In an exemplary embodiment one or both of the phenolic hydroxy groups of the ubiquinol are derivatized with a hydrophilic moiety of the invention. In an exemplary embodiment, the solubilizing agent has a structure according to the Formula (V):

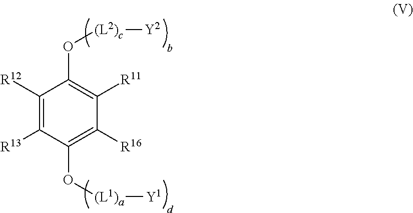

In Formula (V), L¹, L², Y¹ and Y² are defined as herein above. R¹¹, R¹² and R¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁶ is a member selected from OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁷ and R¹⁸ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹² and R¹³, along with the atoms to which they are attached, are optionally joined to form a 4- to 8-membered ring.

In one example, L¹ and L² are linker moieties, which are members independently selected from substituted or unsubstituted alkyl and substituted or unsubstituted heteroalkyl. In another example, Y¹ and Y² are polymeric hydrophilic moieties, which are members independently selected from polyethers, polyalcohols and derivatives thereof. In one embodiment, Y¹, Y², L¹ and L² do not comprise a labeling moiety, a targeting moiety or a drug moiety. The indices a, b, c and d are members independently selected from 0 and 1 with the proviso that at least one of b and d is 1. When b is 0, ((L²)_{c}-Y²)_{b} is preferably a member selected from H, a negative charge, and a salt counterion. When d is 0, ((L¹)ₐ-Y¹)_{d} is preferably a member selected from H, a negative charge, and a salt counterion.

In an exemplary embodiment, in Formula (V), R¹⁶ includes a moiety having a structure selected from the following formulae:

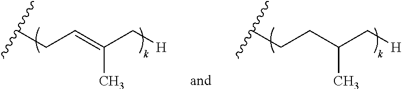

wherein k is an integer selected from 1 to 20. In an exemplary embodiment, k is an integer selected from 6, 7, 8, 9, 10, 11 and 12. In another exemplary embodiment, k is 10.

In an exemplary embodiment, in Formula (V), R¹¹, R¹² and R¹³ are members independently selected from H, unsubstituted alkyl (e.g., methyl, ethyl), alkoxy (e.g., methoxy, t-butoxy), halogen substituted alkoxy and halogen-substituted alkyl (e.g., CF₃). In one example, R¹¹ is H. In another embodiment of the invention, in Formula (V), R¹¹ is a methyl group.

In another exemplary embodiment, one or more of the substituents R¹¹, R¹² and R¹³ include halogen atoms. In another exemplary embodiment the halogen is fluoro. Exemplary fluoroalkyl and fluoroalkoxy groups according to this aspect of the invention include but are not limited to CF₃, OCF₃, CHF₂, OCHF₂, CH₂F, and OCH₂F.

In a particular example, R¹¹ is methyl and R¹² and R¹³ are both methoxy. Hence, in an exemplary embodiment, the solubilizing agent has a structure according to the Formula (VI):

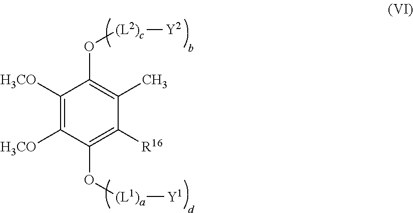

An exemplary solubilizing agent according to Formula (VI) has a structure according to Formula (VII):

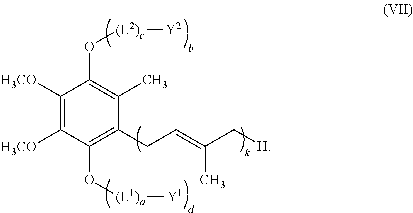

In another exemplary embodiment, one of the phenolic hydroxy groups of the ubiquinol analog is derivatized with a hydrophilic moiety of the invention. Exemplary solubilizing agents have the structure:

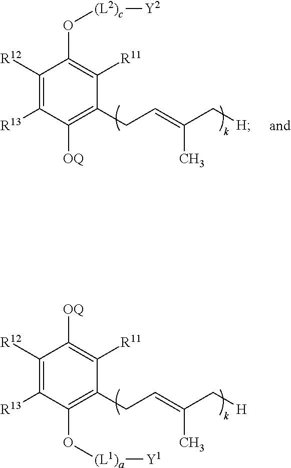

wherein Q is a member selected from H, a negative charge and a salt counter ion.

Exemplary solubilizing agents have a structure according to Formula (VIII), Formula (IX) or Formula (X):

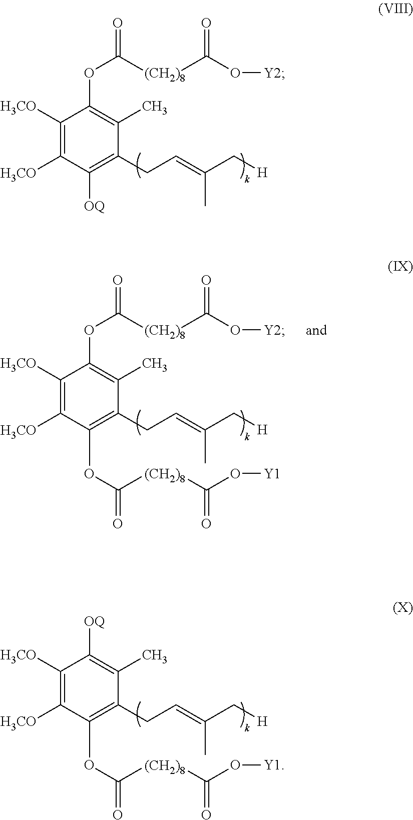

In another exemplary embodiment, one or both of the phenolic hydroxy groups of ubiquinol are part of an ether bond with the linker moiety. Exemplary solubilizing agents have a formula, which is a member selected from:

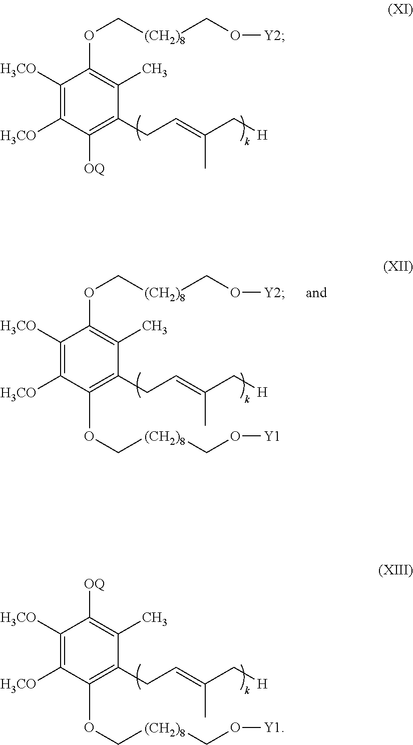

In another exemplary embodiment the invention, the solubilizing agent is a mixture of two or more solubilizing agents described herein. In an exemplary embodiment, the solubilizing agents have a structure according to Formula (V). In one example, the integer k is constant, but at least one of the solubilizing agents includes one hydrophilic moiety, while another includes two hydrophilic moieties. In another embodiment, the mixture includes two regioisomers.

In an exemplary embodiment, the compounds in the mixture of solubilizing agents have structures according to Formulae (VII), (VIII), (IX), (X), (XI), (XII) and (VIII).

Methods of making the above solubilizing agents are known in the art. For example, the methods of making PCS, PTS, and PSS are disclosed in U.S. Pat. Nos. 6,045,826, 6,191,172, 6,632,443, and WO 96/17626, all herein incorporated by reference. The method of making PQS is disclosed in U.S. Patent Application No. 60/915,061, herein incorporated by reference.

### Specific Sterols and Linkers

In an exemplary embodiment, the solubilizing agent has a structure, which is a member selected from:

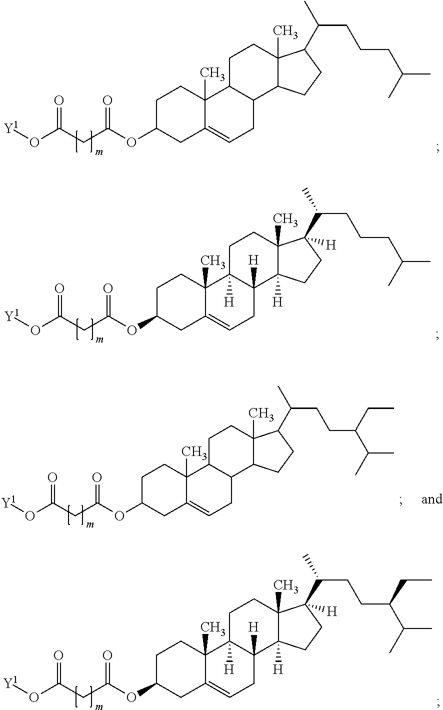

wherein m is a member selected from 2-16. In one example, m is a member selected from 2, 6, 8, 10, 12 and 14. In another example, m is 2. In yet another example, m is 8.

### Specific Sterols and PEG

In an exemplary embodiment, the solubilizing agent is a member selected from

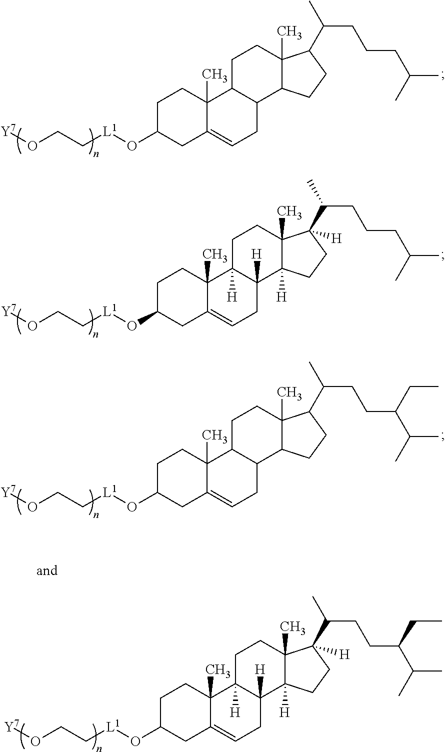

wherein n is a member selected from 10 to 2500, L¹ is a linker moiety, Y⁷ is a member selected from H and methyl.

### Specific Tocopherols and Linkers

In an exemplary embodiment, the solubilizing agent has a structure according to one of the following formulae:

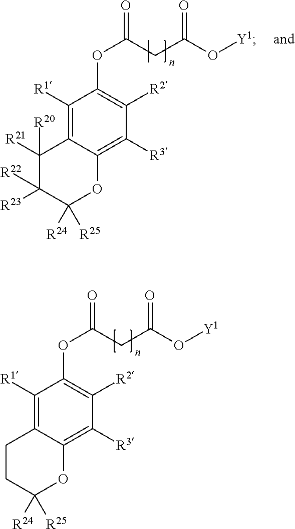

wherein n is an integer selected from 1 to 20. Y¹, R^{1′}, R^{2′}, R^{3′}, R²⁰, R²¹, R²², R²³, R²⁴ and R²⁵ are defined as herein above.

### Specific Tocopherols and PEG

In an exemplary embodiment, the solubilizing agent has a structure according to the following formula:

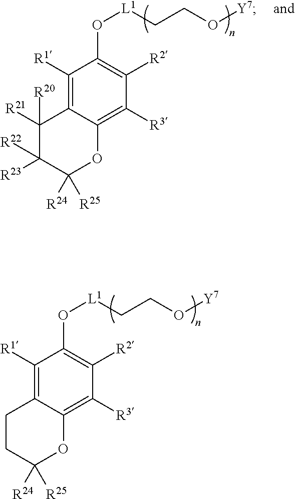

wherein n is a member selected from 10 to 2500. L¹, R^{1′}, R^{2′}, R^{3′}, R²⁰, R²¹, R²², R²³, R²⁴ and R²⁵ are defined as herein above. Y⁷ is selected from H and methyl.

### Specific Ubiquinols and Linkers

In an exemplary embodiment, the solubilizing agent is a member selected from

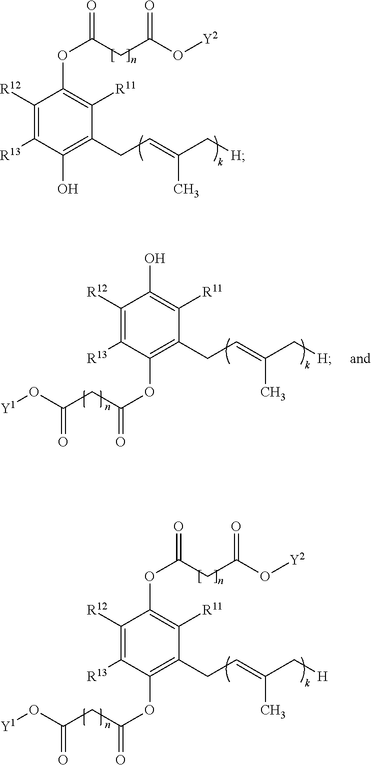

wherein k is a member selected from 1 to 15 and n is a member selected from 1 to 20. Y¹, R¹¹, R¹² and R¹³ are defined as herein above. In an exemplary embodiment, k is 10. In another exemplary embodiment, n is 8.

In an exemplary embodiment, the solubilizing agent is a member selected from

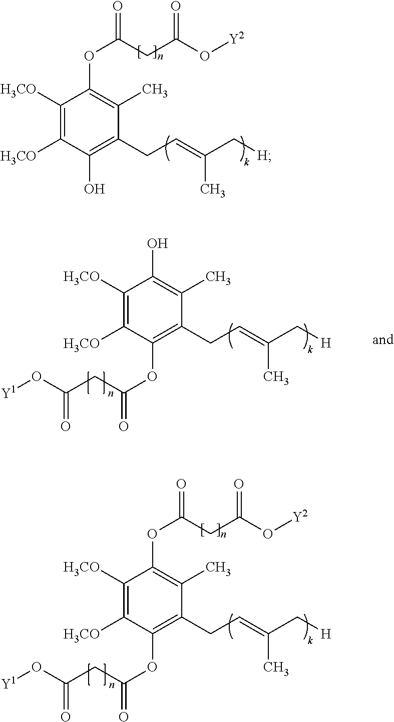

wherein k is a member selected from 1 to 15 and n is a member selected from 1 to 20. Y¹, R¹¹, R¹² and R¹³ are defined as herein above. In an exemplary embodiment, k is 10. In another exemplary embodiment, n is 8.

### Specific Ubiquinols and PEG

In an exemplary embodiment, the solubilizing agent is a member selected from

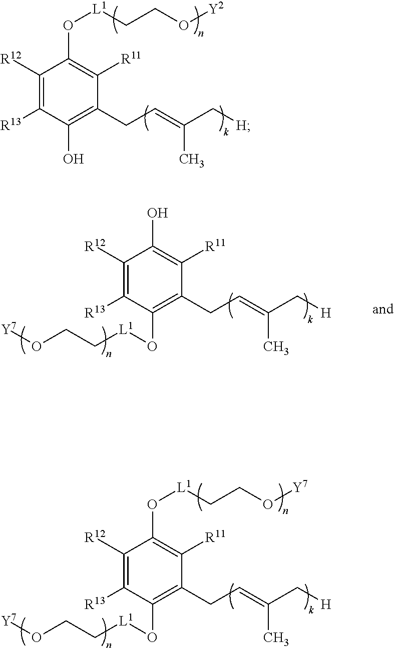

wherein k is a member selected from 1 to 15 and n is a member selected from 10 to 2500. Y¹, R¹¹, R¹² and R¹³ are defined as herein above. In an exemplary embodiment, k is 10. Y⁷ is a member selected from H and methyl.

In an exemplary embodiment, the solubilizing agent is a member selected from

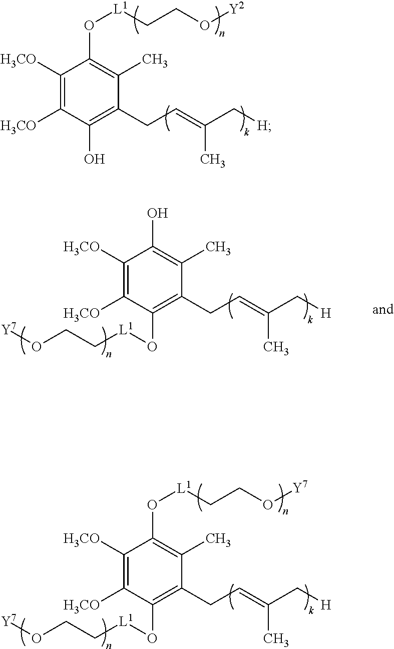

wherein k is a member selected from 1 to 15 and n is a member selected from 10 to 2500. L¹ and Y¹ are defined as herein above. In an exemplary embodiment, k is 10. Y⁷ is a member selected from H and methyl.

In a preferred embodiment, the solubilizing agent is a member selected from polyoxyethanyl-tocopherol-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In an exemplary embodiment, the formulations of the invention include from about 10% to about 50% by weight of a solubilizing agent, such as PTS. Preferably, the formulations include from about 15% to about 40% (w/w) solubilizing agent, more preferably from about 20% to about 40% (w/w), and even more preferably from about 20 to about 35% (w/w).

In an exemplary embodiment, the invention includes from about 0.01% (w/w) to about 5% (w/w) of a solubilizing agent. In an exemplary embodiment, the invention includes from about 0.01% (w/w) to about 0.1% (w/w) of a solubilizing agent. In an exemplary embodiment, the invention includes from about 0.01% (w/w) to about 1% (w/w) of a solubilizing agent. In an exemplary embodiment, the invention includes from about 0.1% (w/w) to about 1% (w/w) of a solubilizing agent. In an exemplary embodiment, the invention includes from about 0.1% (w/w) to about 0.75% (w/w) of a solubilizing agent. In an exemplary embodiment, the invention includes from about 1% (w/w) to about 3% (w/w) of a solubilizing agent. In an exemplary embodiment, the invention includes from about 0.05% (w/w) to about 0.25% (w/w) of a solubilizing agent.

The soft gel capsules of the invention (based on a soft gel capsule weight of from about 900 mg to about 1200 mg) include a solubilizing agent from about 1% to about 30% by weight. In one embodiment, the soft gel capsule includes from about 5% to about 30% (w/w), preferably from about 8% to about 20% of a solubilizing agent, such as PTS.

Solubilizing agents useful in the compositions of the invention include those described in U.S. Provisional Patent Application 60/773,951; and U.S. Pat. No. 6,045,826; 6,191,172 and 6,632,443 to Borowy-Borowski et al., which are incorporated herein by reference for all purposes. These solubilizing agents can be purchased commercially from sources such as Zymes (New Jersey) or produced according to the methods described in the above documents.

### III. (c) Water-Soluble Reducing Agent

In an exemplary embodiment, the water-soluble reducing agent is vitamin C, a water-soluble vitamin C derivative, or a combination thereof. Due to its alpha-keto lactone structure, vitamin C is sensitive to the influence of environmental parameters such as light, heat and oxygen. It is particularly unstable in water or other aqueous solutions. One approach to chemically stabilize the vitamin C molecule is the preparation of ascorbic acid derivatives with greater stability than the parent compound (see, for example, U.S. Pat. Nos. 5,137,723 and 5,078,989), which is incorporated herein by reference.

Hence, in one embodiment, the compositions of the invention include a member selected from ascorbic acid (vitamin C), a vitamin C derivatives, salts thereof and combinations thereof.

In a preferred embodiment, the vitamin C salt, or salt of a vitamin C derivative is an edible (e.g., pharmaceutically acceptable) salt, such as a calcium, sodium, magnesium, potassium and zinc salt. Mixed salts of vitamin C or a vitamin C derivative are also within the scope of the invention.

The compositions of the invention can include one or more vitamin C derivative. The vitamin C derivative can be any analog of vitamin C. Exemplary vitamin C derivative include those in which at least one of the hydroxyl groups of the ascorbic acid molecule (e.g., 2-OH, 3-OH, 5-OH, 6-OH) is derivatized with a modifying group (see e.g., U.S. Pat. No. 5,078,989 to Ando et al.). Alternatively one or more of the hydroxyl group can be substituted with another moiety. In another embodiment, the compositions of the invention include vitamin C as well as at least one vitamin C derivative.

In an exemplary embodiment, the vitamin C or vitamin C derivative is not an ester of ascorbic acid. In another exemplary embodiment, the vitamin C derivative does not have a structure according to the following formula:

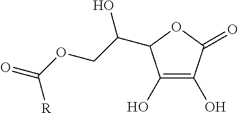

wherein R is at least C₁₅ alkyl (e.g., C₁₅-C₁₈ alkyl). In another exemplary embodiment, the vitamin C derivative is not ascorbyl palmitate.

In order to exhibit stabilizer activity in vitro, the vitamin C derivative can include a free 2-OH and a free 3-OH group. Thus, in a preferred embodiment, the compositions of the invention include at least one vitamin C derivative, in which both the 2-OH and the 3-OH group are non-functionalized. Exemplary vitamin C derivatives according to this embodiment include esters of ascorbic acid, wherein at least one of the 5-OH and the 6-OH group is derivatized.

In one embodiment, the vitamin C ester has a structure according to the following formula:

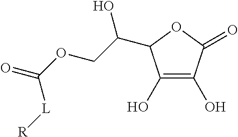

wherein L is a linker group, which is a member selected from a single bond, substituted or unsubstituted alkyl and substituted or unsubstituted heteroalkyl. R is a member selected from substituted or unsubstituted alkyl and substituted or unsubstituted heteroalkyl. In one example, L and R are selected so that the vitamin C derivative retains water-solubility.

Exemplary vitamin C derivatives according to this embodiment include esters, such as 6-O-octanoyl-ascorbic acid, 6-O-dodecanoyl-ascorbic acid, 6-O-tetradecanoyl-ascorbic acid, 6-O-octadecanoyl-ascorbic acid, 6-O-dodecanedioyl-ascorbic acid, 6-O-docosanedioyl-ascorbic acid, 6-O-thapsoyl-ascorbic acid, 6-O-suberoyl-ascorbic acid, 6-O-adipoyl-ascorbic acid. Other examples include those esters, in which the lipophilic part of the molecule represents a mono- or polyunsaturated fatty acid. In one embodiment, the unsaturated fatty acid is an essential fatty acid associated with a health benefit (e.g., human health), such as an omega-3 (alpha-linolenic acid), omega-6 or omega-9 fatty acid. Other examples include esters of vitamin C including an amino acid residue. In an exemplary embodiment, the vitamin C derivative has a structure according to the following formula:

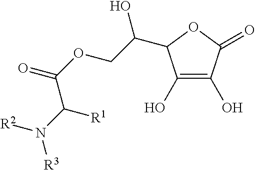

wherein R¹ is an amino acid side chain. R² and R³ are members independently selected from H, substituted or unsubstituted alkyl and substituted or unsubstituted heteroalkyl.

In another embodiment, the compositions of the invention include 2-O-alkyl or 3-O-alkyl derivatives of vitamin C. 3-O-alkyl-ascorbic acids have been reported by Nihro et al., Chem. Pharm. Bull. 1991, 39: 1731-1735, the disclosure of which is incorporated herein by reference.

In yet another embodiment, the vitamin C derivative is a glucoside of ascorbic acid, such as ascorbic acid 1-glucoside, ascorbic acid 2-glucoside, ascorbic acid 3-glucoside, ascorbic acid 5-glucoside, and ascorbic acid 6-glucoside. Examples include 2-O-(alpha-D-glucopyranosyl)-ascorbic acid (see, e.g., U.S. Pat. No. 5,137,723) and 2-O-(beta-D-glucopyranosyl)-ascorbic acid (see e.g., U.S. Patent Application No. 2005/0113312). Also within the scope of the invention are difunctionalized derivatives of vitamin C, such as e.g., 6-O-acyl-2-O-(alpha-D-glucopyranosyl) ascorbic acids (see e.g., Yamamoto et al., J. Med. Chem. 2002, 45(2): 462-468. The above references are incorporated herein by reference.

In a further embodiment, the vitamin C derivative is a phosphate of ascorbic acid. In an exemplary embodiment the ascorbyl phosphate is a salt of an alkali metal, an alkaline earth metal, or a transition metal. Preferred examples include magnesium ascorbyl phosphate, sodium ascorbyl phosphate (e.g., sodium salt of ascorbyl-2-monophosphate), calcium ascorbyl phosphate, potassium ascorbyl phosphate and mixed salts, such as e.g., sodium magnesium ascorbyl phosphate or sodium calcium ascorbyl phosphate, aminopropyl ascorbyl phosphate. The ascorbyl phosphate can exist as a hydrate, wherein dihydrates are common. An exemplary dihydrate is available for example from DSM under the product name STAY-C 50.

Other art-recognized vitamin C derivatives are also useful for the purpose of this invention.

In an exemplary embodiment, the stabilizer is in excess in relation to the lipophilic bioactive molecule. In another exemplary embodiment, the ratio of the lipophilic bioactive molecule to said stabilizer is from about 1:1 (w/w) to about 1:6 (w/w). In another exemplary embodiment, the ratio of the lipophilic bioactive molecule to said stabilizer is from about 1:1 (w/w) to about 1:5 (w/w). In another exemplary embodiment, the ratio of the lipophilic bioactive molecule to said stabilizer is from about 1:1.3 (w/w) to about 1:3 (w/w). In another exemplary embodiment, the ratio of the lipophilic bioactive molecule to said stabilizer is from about 1:2 (w/w) to about 1:4 (w/w). In another exemplary embodiment, the ratio of the lipophilic bioactive molecule to said stabilizer is about 1:3 (w/w).

In an exemplary embodiment, the stabilizer is vitamin C or a vitamin C derivative. In one example, the vitamin C or the vitamin C derivative is used in a molar excess in relation to the lipophilic bioactive molecule. In another exemplary embodiment, the ratio of the lipophilic bioactive molecule to said vitamin C or vitamin C derivative is from about 1:1 (w/w) to about 1:6 (w/w). In another exemplary embodiment, the ratio of the lipophilic bioactive molecule to said vitamin C or vitamin C derivative is from about 1:1 (w/w) to about 1:10 (w/w). In another exemplary embodiment, the ratio of the lipophilic bioactive molecule to said vitamin C or vitamin C derivative is from about 1:1.3 (w/w) to about 1:5 (w/w). In another exemplary embodiment, the ratio of the lipophilic bioactive molecule to said vitamin C or vitamin C derivative is from about 1:2 (w/w) to about 1:4 (w/w). In another exemplary embodiment, the ratio of the lipophilic bioactive molecule to said vitamin C or vitamin C derivative is about 1:3 (w/w).

### III. (d) Other Components

The formulations described herein (either aqueous or non-aqueous) can further include various ingredients useful to stabilize the composition, promote the bioavailability of the lipophilic bioactive molecule, or provide nutritional value.

Exemplary additives of the present formulations include, without limitation, pharmaceutical drug molecules, antibiotics, sterols, vitamins, provitamins, carotenoids (e.g., alpha and beta-carotenes, cryptoxanthin, lutein and zeaxanthin), phospholipids, L-camitine, starches, sugars, fats, stabilizers, reducing agents, free radical scavengers, amino acids, amino acid analogs, proteins, solvents, emulsifiers, adjuvants, sweeteners, fillers, flavoring agents, coloring agents, lubricants, binders, moisturizing agents, preservatives, suspending agents, starch, hydrolyzed starch(es), derivatives thereof and combinations thereof.

In an exemplary embodiment, the formulation further comprises gelatin. In an exemplary embodiment, the formulation further comprises sorbitol. In an exemplary embodiment, the formulation further comprises glycerin. In an exemplary embodiment, the formulation further comprises purified water. In an exemplary embodiment, the formulation further comprises polysorbate 80. In an exemplary embodiment, the formulation further comprises hydroxylated lecitin. In an exemplary embodiment, the formulation further comprises medium chain triglycerides. In an exemplary embodiment, the formulation further comprises annato seed extract. In an exemplary embodiment, the formulation further comprises soybean oil. In an exemplary embodiment, the formulation further comprises omega-3 enriched fish oil. In an exemplary embodiment, the formulation further comprises rice bran oil. In an exemplary embodiment, the formulation further comprises carotenoids. In an exemplary embodiment, the formulation further comprises titanium dioxide. In an exemplary embodiment, the formulation further comprises suspending agents such as silica (silicon dioxide). In an exemplary embodiment, the formulation further comprises riboflavin. Various other additives can be incorporated into the present formulations including, without limitation, phospholipids, L-carnitine, anti-inflammatory agents, anti-aging agents, starches, sugars, fats, stabilizers, amino acids, proteins, flavorings, coloring agents, hydrolyzed starch(es) and derivatives thereof (such as time release esters (Ester-C, Ester-E)) or combinations thereof. Anti-inflammatory agents of use in the invention include, but are not limited to, bisabolol, mentholatum, dapsone, aloe, hydrocortisone, and the like. Anti-aging agents of use in the invention include, but are not limited to, niacinamide, retinol and retinoid derivatives, AHA, lipoic acid, beta hydroxy acids, salicylic acid, copper binding peptides and the like.

The formulations described herein can also include vitamins and biologically-acceptable minerals. Non-limiting examples of vitamins include vitamin A, B vitamins, vitamin C, vitamin D, vitamin E, vitamin K and folic acid. Vitamin derivatives can also be added to the formulations of the invention, such as tazarotene, calcipotriene, tretinoin, adapalene and the like. The Vitamin E family includes a family of four compounds (forms) of tocopherols (alpha, beta, delta, and gamma) and four compounds of tocotrienols (alpha, beta, delta, and gamma). Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s) in a unit dosage form of the invention are present in amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s) in a unit dosage form of the invention are present in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

In the formulations of the invention the additional components are usually a minor component (from about 0.001% to about 20% by weight or preferably from about 0.01% to about 10% by weight) with the remainder being various vehicles or carriers and processing aids helpful for forming the desired dosing form.

### IV. Pharmaceutical Formulations

According to another aspect, the invention provides pharmaceutical formulations comprising a formulation of the invention and a pharmaceutically acceptable carrier. Pharmaceutical formulations include nutracetical formulations.

An exemplary unit dosage form (e.g., contained in a soft gel capsule) of the invention includes a lipophilic bioactive molecule (ubiquinol, DHA, astaxanthin) in an amount of about 1% to about 30% by weight. In one embodiment, the unit dosage form (e.g., soft gel capsule) includes from about 3% to about 20% (w/w), and preferably from about 5% to about 20% of a lipohilic bioactive molecule. Typically, soft-gel formulations include from about 5% to about 30% (w/w) of lipophilic bioactive molecule, from about 15% to about 40% (w/w) solubilizing agent (e.g., PQS, PTS), from about 30% to about 60% (w/w) lipophilic carrier (e.g., fish oil) and from about 1% to about 10% (w/w) viscosity enhancer (e.g., beeswax). In an exemplary embodiment, the soft gel capsule of the invention includes ubiquinol, vitamin C, solubilizing agent (e.g., PTS or PQS), beeswax and a lipophilic carrier (e.g., fish oil) enriched with omega fatty acid.

In an exemplary embodiment, the lipophilic bioactive molecule is combined with a solubilizing agent useful to improve the bioavailability of the lipophilic bioactive molecule. Such formulations may further contain additional active ingredients and/or pharmaceutically or cosmetically acceptable additives or vehicles, including solvents, adjuvants, excipients, sweeteners, fillers, colorants, flavoring agents, lubricants, binders, moisturizing agents, preservatives and mixtures thereof. The formulations may be suitable for topical (e.g., a cream, lotion, gel, ointment, dermal adhesive patch), oral (e.g., a capsule, tablet, caplet, granulate), or parenteral (e.g., suppository, sterile solution) administration. Among the acceptable vehicles and solvents that may be employed for administration by injection are water, mildly acidified water (e.g. acidified carbonated water), Ringer's solution and isotonic sodium chloride solution.

In some embodiments, the formulation is in the form of a drinkable liquid or sirup and can be formulated in a mildly acidified water (e.g. acidified carbonated water) as the carrier.

Ubiquinone or ubiquinol, when combined with a solubilizing agent of the invention, can be administered to a warm-blooded animal, particularly a human, in need of the prophylaxis or therapy. The method comprises administering to such human or warm-blooded animal, an effective amount of a water-soluble formulation of the invention.

When the hydrophobic moiety of the solubilizing agent is linked to the hydrophilic moiety through a linker, which is cleavable in vivo, the formulation can provide an additional benefit for the patient. In vivo, the solubilizing agent is hydrolyzed by enzymes and is systemically converted back to the respective ubiquinol or tocopherol.

The pharmaceutical composition can be prepared according to known methods. Formulations are described in detail in a number of sources, which are well known and readily available to those skilled in the art. For example, Remington's Pharmaceutical Science by E. W. Martin describes formulation, which can be used in connection with the subject invention. In general, the compositions of the subject invention are formulated such that an effective amount of the lipophilic bioactive molecule is provided in the composition.

In accordance with the present invention, pharmaceutical compositions are provided which comprise, an active ingredient as described, supra, and an effective amount of one or more pharmaceutically acceptable excipients, vehicles, carriers or diluents. Examples of such carriers include ethanol, dimethyl sulfoxide, glycerol, silica, alumina, starch, and equivalent carriers and diluents. Further, acceptable carriers can be either solid or liquid. Solid form preparations include powders, tablets, pills, capsules, cachets, suppositories and dispersible granules. A solid carrier can be one or more substances, which may act as diluents, flavoring agents, solubilizing agents, lubricants, suspending agents, binders, preservatives, tablet disintegrating agents or encapsulating materials.

Injectable preparations include sterile suspensions, solutions or emulsions of the active ingredient in aqueous or oily vehicles. The compositions can also contain formulating agents, such as suspending, stabilizing and/or dispersing agent. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, dextrose solution, etc., before use. To this end, the compositions can be lyophilized. The stored preparations can be supplied in unit dosage forms and reconstituted prior to use in vivo.

Alternatively, transdermal delivery systems manufactured as an adhesive disc or patch which slowly releases the active ingredient for percutaneous absorption can be used. To this end, permeation enhancers can be used to facilitate transdermal penetration of the active ingredient. A particular benefit can be achieved by incorporating the active agents of the invention into a nitroglycerin patch for use in patients with ischemic heart disease and hypercholesterolemia.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For buccal administration, the compositions can take the form of tablets or lozenges formulated in conventional manner

For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The disclosed pharmaceutical compositions can be subdivided into unit doses containing appropriate quantities of the active component. The unit dosage form can be a packaged preparation, such as packeted tablets, capsules, and powders in paper or plastic containers or in vials or ampoules. Also, the unit dosage can be a liquid based preparation or formulated to be incorporated into solid food products, chewing gum, or lozenges.

Pharmaceutically acceptable salts (counter ions) can be conveniently prepared by ion-exchange chromatography or other methods as are well known in the art.

The formulations of the invention can take a variety of forms adapted to the chosen route of administration. Those skilled in the art will recognize various synthetic methodologies that may be employed to prepare non-toxic pharmaceutical formulations incorporating the compounds described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The compositions of the invention may be administered orally, topically, parenterally, by inhalation or spray or rectally in dosage unit formulations containing conventional non-toxic pharmaceutically acceptable carriers, adjuvants and vehicles. It is further understood that the best method of administration may be a combination of methods. The term parenteral as used herein includes subcutaneous injections, intradermal, intravascular (e.g., intravenous), intramuscular, spinal, intrathecal injection or like injection or infusion techniques.

The formulations are preferably in a form suitable for oral use, for example, as tablets, troches, lozenges, aqueous or oily suspensions, dispersible powders or granules, emulsion, hard or soft capsules, soft gel capsules, or syrups or elixirs.

The formulations described herein may be prepared according to any method known in the art for the manufacture of pharmaceutical formulations and nutriceuticals, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Formulations of the invention may also be in the form of oil-in-water emulsions and water-in-oil emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The formulations may be in the form of a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For administration to non-human animals, the formulations of the invention may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also be added as a food or drink supplement for humans.

Dosage levels (with respect to lipophilic bioactive molecule) of the order of from about 1 mg to about 250 mg per kilogram of body weight per day are useful. For example, a dosage level from about 25 mg to about 150 mg per kilogram of body weight per day, are useful. The amount of active ingredient that may be combined with the carrier materials to produce a single dosage form will vary depending upon the condition being treated and the particular mode of administration. Dosage unit forms will generally contain between from about 1 mg to about 500 mg of the lipophilic bioactive molecule (e.g., ubiquinol, omega-3-fatty acids (e.g., ALA, DHA) and carotenoids (e.g., astaxanthin, fucoxanthin, cantaxanthin and the like). For example, dosage unit forms of about 1 mg to about 250 mg, about 1 mg to about 100 mg or 1 mg to about 80, 60, 40, 20 or 10 mg are useful.

Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

The present invention also provides packaged formulations of the invention and instructions for use of the tablet, capsule, soft gel capsule, elixir, etc. Typically, the packaged formulation, in whatever form, is administered to an individual in need thereof that requires an increase in the amount of ubiquinone/ubiquinol in the individual's diet. Typically, the dosage requirement is between about 1 to about 4 dosages a day.

### Soft Gel Capsules

In an exemplary embodiment, the formulation of the invention is encapsulated within a soft gelatin (soft gel) capsule. In another exemplary embodiment, the solvent within the capsule is propylene glycol. In another exemplary embodiment, the capsule is essentially free of precipitation within said capsule. In another exemplary embodiment, the capsule is essentially free of precipitated ubiquinone/ubiquinol within said capsule. In another exemplary embodiment, the ubiquinone/ubiquinol is CoQ₁₀, vitamin C or vitamin C derivative, and the solubilizing agent is PTS. In another exemplary embodiment, the ratio of said ubiquinone/ubiquinol to said PTS is from about 1:1 to about 1:2.

Soft gel or soft gelatin capsules can be prepared, for example, without limitation, by dispersing the formulation in an appropriate vehicle (e.g. fish or marine oil, rice bran oil, monoterpene and/or beeswax) to form a high viscosity mixture. This mixture is then encapsulated with a gelatin based film using technology and machinery known to those in the soft gel industry. The industrial units so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing soft gelatin shells, the shell can include between about 20 to 70 percent gelatin, generally a plasticizer and about 5 to about 60% by weight sorbitol. The filling of the soft gelatin capsule is liquid (principally limonene, in combination with fish or marine oil, rice bran oil and/or beeswax if desired) and can include, apart from the stabilizer actives, a hydrophilic matrix. The hydrophilic matrix, if present, is a polyethylene glycol having an average molecular weight of from about 200 to 1000. Further ingredients are optionally thickening agents. In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerin, water and various additives. Typically, the percentage (by weight) of the gelatin is between about 30 and about 50 weight percent, in particular between about 35 and about weight percent and more specifically about 42 weight percent. The formulation includes between about 15 and about 25 weight percent glycerin, more particularly between about 17 and about 23 weight percent and more specifically about 20 weight percent glycerin.

The remaining portion of the capsule is typically water. The amount varies from between about 25 weigh percent and about 40 weight percent, more particularly between about 30 and about 35 weight percent, and more specifically about 35 weight percent. The remainder of the capsule can vary, generally, between about 2 and about 10 weight percent composed of a flavoring agent(s), sugar, coloring agent(s), etc. or combination thereof. After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

Typically, when a soft gel capsule is prepared, the total weight is between about 250 milligrams and about 2.5 gram in weight, e.g., 400-750 milligrams. Therefore, the total weight of additives, such as vitamins and stabilizers, is between about 80 milligrams and about 2000 milligrams, alternatively, between about 100 milligrams and about 1500 milligrams, and in particular between about 120 milligrams and about 1200 milligrams. In particular, the soft gel capsule typically weighs between about 1000 milligrams and 1300 milligrams, wherein the percentage fill is about 50% of the entire weight of the capsule, i.e., from about 450 to about 800 milligrams fill weight. The fill weight includes the active ingredient(s), solubilizing agents, etc.

Preparation of the soft gel capsules was accomplished by methods well known in the art including, but not limited to those described throughout the specification and in U.S. Pat. Nos. 6,616,942, 6,623,734 and pending U.S. Ser. Nos. 10/035,753 and 09/825,920, the contents of which are incorporated herein by reference in their entirety. An exemplary method of making soft gel capsules is disclosed in U.S. Patent Application No. 60/886,212, which is herein incorporated by reference.

### Exemplary Formulations not Including a Stabilizer

In one aspect, the invention provides a formulation which comprises: (a) a lipophilic bioactive molecule; (b) a solubilizing agent; and does not include a stabilizer. In an exemplary embodiment, the ratio of the lipophilic bioactive molecule to said solubilizing agent is from about 1:0.3 (w/w) to about 1:20 (w/w). In an exemplary embodiment, the ratio of the lipophilic bioactive molecule to said solubilizing agent is from about 1:1 (w/w) to about 1:20 (w/w). In another exemplary embodiment, the ratio of the lipophilic bioactive molecule to said solubilizing agent is from about 1:1 (w/w) to about 1:10 (w/w). In another exemplary embodiment, the ratio of the lipophilic bioactive molecule to said solubilizing agent is from about 1:1.3 (w/w) to about 1:5 (w/w). In another exemplary embodiment, the ratio of the lipophilic bioactive molecule to said solubilizing agent is from about 1:2 (w/w) to about 1:4 (w/w). In another exemplary embodiment, the ratio of the lipophilic bioactive molecule to said solubilizing agent is about 1:3 (w/w). In an exemplary embodiment, the ratio of the lipophilic bioactive molecule to said solubilizing agent is from about 1:0.3 (w/w) to about 1:1 (w/w). In an exemplary embodiment, the ratio of the lipophilic bioactive molecule to said solubilizing agent is from about 1:0.5 (w/w) to about 1:2 (w/w).

In one example, the solubilizing agent is PTS. In an exemplary embodiment, the ratio of the lipophilic bioactive molecule to PTS is from about 1:0.3 (w/w) to about 1:20 (w/w). In an exemplary embodiment, the ratio of the lipophilic bioactive molecule to PTS is from about 1:1 (w/w) to about 1:20 (w/w). In another exemplary embodiment, the ratio of the lipophilic bioactive molecule to PTS is from about 1:1 (w/w) to about 1:10 (w/w). In another exemplary embodiment, the ratio of the lipophilic bioactive molecule to PTS is from about 1:1.3 (w/w) to about 1:5 (w/w). In another exemplary embodiment, the ratio of the lipophilic bioactive molecule to PTS is from about 1:2 (w/w) to about 1:4 (w/w). In another exemplary embodiment, the ratio of the lipophilic bioactive molecule to PTS is about 1:3 (w/w). In an exemplary embodiment, the ratio of the lipophilic bioactive molecule to PTS is from about 1:0.3 (w/w) to about 1:1 (w/w). In an exemplary embodiment, the ratio of the lipophilic bioactive molecule to PTS is from about 1:0.5 (w/w) to about 1:2 (w/w).

In an exemplary embodiment, the invention provides a formulation which comprises: (a) a ubiquinone; (b) a solubilizing agent (e.g., PTS) and does not include a stabilizer.

In an exemplary embodiment, the invention provides a formulation which comprises: (a) a ubiquinol; (b) a solubilizing agent (e.g., PTS); and does not include a stabilizer.

In an exemplary embodiment, the invention provides a formulation which comprises: (a) resveratrol; (b) a solubilizing agent (e.g., PTS); and does not include a stabilizer.

In an exemplary embodiment, the invention provides a formulation which comprises: (a) alpha-carotene; (b) a solubilizing agent (e.g., PTS); and does not include a stabilizer.

In an exemplary embodiment, the invention provides a formulation which comprises: (a) beta-carotene; (b) a solubilizing agent (e.g., PTS); and does not include a stabilizer.

In an exemplary embodiment, the invention provides a formulation which comprises: (a) lycopene; (b) a solubilizing agent (e.g., PTS); and does not include a stabilizer.

In an exemplary embodiment, the invention provides a formulation which comprises: (a) lutein; (b) a solubilizing agent (e.g., PTS); and does not include a stabilizer.

In an exemplary embodiment, the invention provides a formulation which comprises: (a) astaxanthin; (b) a solubilizing agent (e.g., PTS); and does not include a stabilizer.

In an exemplary embodiment, the invention provides a formulation which comprises: (a) canthaxanthin; (b) a solubilizing agent (e.g., PTS); and does not include a stabilizer.

In an exemplary embodiment, the invention provides a formulation which comprises: (a) fucoxanthin; (b) a solubilizing agent (e.g., PTS); and does not include a stabilizer.

In an exemplary embodiment, the invention provides a formulation which comprises: (a) violaxanthin; (b) a solubilizing agent (e.g., PTS); and does not include a stabilizer.

In an exemplary embodiment, the invention provides a formulation which comprises: (a) asiatic acid; (b) a solubilizing agent (e.g., PTS); and does not include a stabilizer.

In an exemplary embodiment, the invention provides a formulation which comprises: (a) β-sitosterol; (b) a solubilizing agent (e.g., PTS); and does not include a stabilizer.

In an exemplary embodiment, the invention provides a formulation which comprises: (a) β, γ or Δ-tocopherol; (b) a solubilizing agent (e.g., PTS); and does not include a stabilizer.

In an exemplary embodiment, the invention provides a formulation which comprises: (a) alpha-tocopherol; (b) a solubilizing agent (e.g., PTS); and does not include a stabilizer.

In an exemplary embodiment, the invention provides a formulation which comprises: (a) alpha-tocotrienol; (b) a solubilizing agent (e.g., PTS); and does not include a stabilizer.

In an exemplary embodiment, the invention provides a formulation which comprises: (a) linolenic acid; (b) a solubilizing agent (e.g., PTS); and does not include a stabilizer.

In an exemplary embodiment, the invention provides a formulation which comprises: (a) linoleic acid; (b) a solubilizing agent (e.g., PTS); and does not include a stabilizer.

In an exemplary embodiment, the invention provides a formulation which comprises: (a) oleic acid; (b) a solubilizing agent (e.g., PTS); and does not include a stabilizer.

In an exemplary embodiment, the invention provides a formulation which comprises: (a) gamma linolenic acid; (b) a solubilizing agent (e.g., PTS); and does not include a stabilizer.

In an exemplary embodiment, the invention provides a formulation which comprises: (a) docosahexaenoic acid; (b) a solubilizing agent (e.g., PTS); and does not include a stabilizer.

In an exemplary embodiment, the invention provides a formulation which comprises: (a) eicosapentaenoic acid; (b) a solubilizing agent (e.g., PTS); and does not include a stabilizer.

In an exemplary embodiment, the invention provides a formulation which comprises: (a) alpha-linolenic acid; (b) a solubilizing agent (e.g., PTS); and does not include a stabilizer.

In an exemplary embodiment, the invention provides a formulation which comprises: (a) alpha-lipoic acid; (b) a solubilizing agent (e.g., PTS); and does not include a stabilizer.

In an exemplary embodiment, the invention provides a formulation which comprises: (a) glutathione; (b) a solubilizing agent (e.g., PTS); and does not include a stabilizer.

In an exemplary embodiment, the invention provides a formulation which comprises: (a) a flavoring oil; (b) a solubilizing agent (e.g., PTS); and does not include a stabilizer.

In one example according to any of the above embodiments, the stabilizer which is not included in the formulation is vitamin C, a vitamin C derivative, or a combination thereof. In an exemplary embodiment, the stabilizer which is not included in the formulation is ascorbyl palmitate. In an exemplary embodiment, the stabilizer which is not included in the formulation is an ascorbyl moiety with a fatty acid counterion. In an exemplary embodiment, the lipophilic bioactive molecule is present in said formulation in an amount equivalent to at least 0.5% (w/w), at least 1% (w/w), at least 1.5% (w/w), at least 2%, at least 2.5%, at least 3%, at least 3.5%, at least 4%, at least 4.5% or at least 5% (w/w). In another example according to any of the above embodiments, the stabilizer is a member selected from PTS, PQS, PCS and PSS.

### Exemplary Formulations Including Stabilizers

In an exemplary embodiment, the invention provides a formulation which comprises: (a) a ubiquinone; (b) a solubilizing agent (e.g., PTS); and (c) a water-soluble reducing agent (stabilizer) (e.g., vitamin C, a vitamin C derivative or mixtures thereof). In an exemplary embodiment, the ratio of the lipophilic bioactive molecule to said solubilizing agent is from about 1:0.3 (w/w) to about 1:20 (w/w). In an exemplary embodiment, the ratio of the lipophilic bioactive molecule to said solubilizing agent is from about 1:1 (w/w) to about 1:20 (w/w). In another exemplary embodiment, the ratio of the lipophilic bioactive molecule to said solubilizing agent is from about 1:1 (w/w) to about 1:10 (w/w). In another exemplary embodiment, the ratio of the lipophilic bioactive molecule to said solubilizing agent is from about 1:1.3 (w/w) to about 1:5 (w/w). In another exemplary embodiment, the ratio of the lipophilic bioactive molecule to said solubilizing agent is from about 1:2 (w/w) to about 1:4 (w/w). In another exemplary embodiment, the ratio of the lipophilic bioactive molecule to said solubilizing agent is about 1:3 (w/w). In an exemplary embodiment, the ratio of the lipophilic bioactive molecule to said solubilizing agent is from about 1:0.3 (w/w) to about 1:1 (w/w). In an exemplary embodiment, the ratio of the lipophilic bioactive molecule to said solubilizing agent is from about 1:0.5 (w/w) to about 1:2 (w/w).

In another exemplary embodiment, the ratio of said ubiquinone/ubiquinol to said PTS is from about 1:2 to about 1:4. In another exemplary embodiment, the ratio of said ubiquinone/ubiquinol to said PTS is about 1:3. In another exemplary embodiment, the formulation is an aqueous solution that includes CoQ₁₀ or a reduced form thereof, PTS, wherein the ratio of said ubiquinone/ubiquinol to said PTS is from about 1:2 to about 1:4, and an excess of a Vitamin C or Vitamin C derivative. In another exemplary embodiment, the formulation is a soft gel capsule which includes CoQ₁₀ or a reduced form thereof, PTS, wherein the ratio of said ubiquinone/ubiquinol to said PTS is from about 1:2 to about 1:4 and an excess of a Vitamin C or Vitamin C derivative.

In an exemplary embodiment, the invention provides a formulation which comprises: (a) a ubiquinone; (b) a solubilizing agent (e.g., PTS); and vitamin C, a vitamin C derivative, or combinations thereof. In an exemplary embodiment, the stabilizer is not ascorbyl palmitate. In an exemplary embodiment, the invention provides a formulation, which comprises: (a) an ubiquinone; (b) PTS; and (c) vitamin C and/or a Vitamin C derivative or combinations thereof, wherein said Vitamin C derivative is not ascorbyl palmitate. In an exemplary embodiment, the invention provides a formulation which comprises: (a) an ubiquinone; (b) PTS; and (c) Vitamin C and/or a Vitamin C derivative, or combinations thereof. In an exemplary embodiment, the ubiquinone is CoQ₁₀. In yet another exemplary embodiment, the ubiquinone is present in said formulation in an amount of at least about 0.5% by weight, at least about 1% by weight, at least about 1.5% by weight, at least about 2% by weight, at least about 2.5% by weight, at least about 3% by weight, at least about 3.5% by weight, at least about 4% by weight, at least about 4.5% by weight or at least about 5% by weight. In an exemplary embodiment, the ubiquinone is present in said formulation in an amount of at least about 95% by weight, at least about 96% by weight or at least about 97% by weight.

In an exemplary embodiment, the invention provides a formulation which comprises: (a) a ubiquinol; (b) a solubilizing agent (e.g., PTS); and (c) a stabilizer. In an exemplary embodiment, the invention provides a formulation which comprises: (a) a ubiquinone; (b) a solubilizing agent; and (c) vitamin C, a vitamin C derivative, or combinations thereof. In an exemplary embodiment, the invention provides a formulation which comprises: (a) an ubiquinol; (b) PTS; and (c) vitamin C, a vitamin C derivative, or combinations thereof.

In an exemplary embodiment, the invention provides a formulation which comprises: (a) resveratrol; (b) a solubilizing agent (e.g., PTS); and (c) a stabilizer. In an exemplary embodiment, the invention provides a formulation which comprises: (a) resveratrol; (b) a solubilizing agent; and (c) vitamin C, a vitamin C derivative, or combinations thereof. In an exemplary embodiment, the invention provides a formulation which comprises: (a) resveratrol; (b) PTS; and (c) vitamin C, a vitamin C derivative, or combinations thereof.

In an exemplary embodiment, the invention provides a formulation which comprises: (a) alpha-carotene; (b) a solubilizing agent (e.g., PTS); and (c) a stabilizer. In an exemplary embodiment, the invention provides a formulation which comprises: (a) alpha-carotene; (b) a solubilizing agent; and (c) vitamin C, a vitamin C derivative, or combinations thereof. In an exemplary embodiment, the invention provides a formulation which comprises: (a) alpha-carotene; (b) PTS; and (c) vitamin C, a vitamin C derivative, or combinations thereof.

In an exemplary embodiment, the invention provides a formulation which comprises: (a) beta-carotene; (b) a solubilizing agent (e.g., PTS); and (c) a stabilizer. In an exemplary embodiment, the invention provides a formulation which comprises: (a) beta-carotene; (b) a solubilizing agent; and (c) Vitamin C, a Vitamin C derivative, or combinations thereof. In an exemplary embodiment, the invention provides a formulation which comprises: (a) beta-carotene; (b) PTS; and (c) Vitamin C, a Vitamin C derivative, or combinations thereof

In an exemplary embodiment, the invention provides a formulation which comprises: (a) lycopene; (b) a solubilizing agent (e.g., PTS); and (c) a stabilizer. In an exemplary embodiment, the invention provides a formulation which comprises: (a) lycopene; (b) a solubilizing agent; and (c) Vitamin C, a Vitamin C derivative, or combinations thereof. In an exemplary embodiment, the invention provides a formulation which comprises: (a) lycopene; (b) PTS; and (c) Vitamin C, a Vitamin C derivative, or combinations thereof.

In an exemplary embodiment, the invention provides a formulation which comprises: (a) lutein; (b) a solubilizing agent (e.g., PTS); and (c) a stabilizer. In an exemplary embodiment, the invention provides a formulation which comprises: (a) lutein; (b) a solubilizing agent; and (c) Vitamin C, a Vitamin C derivative, or combinations thereof. In an exemplary embodiment, the invention provides a formulation which comprises: (a) lutein; (b) PTS; and (c) Vitamin C, a Vitamin C derivative, or combinations thereof.

In an exemplary embodiment, the invention provides a formulation which comprises: (a) astaxanthin; (b) a solubilizing agent (e.g., PTS); and (c) a stabilizer. In an exemplary embodiment, the invention provides a formulation which comprises: (a) astaxanthin; (b) a solubilizing agent; and (c) Vitamin C, a Vitamin C derivative, or combinations thereof. In an exemplary embodiment, the invention provides a formulation which comprises: (a) astaxanthin; (b) PTS; and (c) Vitamin C, a Vitamin C derivative, or combinations thereof.

In an exemplary embodiment, the invention provides a formulation which comprises: (a) canthaxanthin; (b) a solubilizing agent (e.g., PTS); and (c) a stabilizer. In an exemplary embodiment, the invention provides a formulation which comprises: (a) canthaxanthin; (b) a solubilizing agent; and (c) Vitamin C, a Vitamin C derivative, or combinations thereof. In an exemplary embodiment, the invention provides a formulation which comprises: (a) canthaxanthin; (b) PTS; and (c) Vitamin C, a Vitamin C derivative, or combinations thereof.

In an exemplary embodiment, the invention provides a formulation which comprises: (a) fucoxanthin; (b) a solubilizing agent (e.g., PTS); and (c) a stabilizer. In an exemplary embodiment, the invention provides a formulation which comprises: (a) fucoxanthin; (b) a solubilizing agent; and (c) Vitamin C, a Vitamin C derivative, or combinations thereof. In an exemplary embodiment, the invention provides a formulation which comprises: (a) fucoxanthin; (b) PTS; and (c) Vitamin C, a Vitamin C derivative, or combinations thereof.

In an exemplary embodiment, the invention provides a formulation which comprises: (a) violaxanthin; (b) a solubilizing agent (e.g., PTS); and (c) a stabilizer. In an exemplary embodiment, the invention provides a formulation which comprises: (a) violaxanthin; (b) a solubilizing agent; and (c) Vitamin C, a Vitamin C derivative, or combinations thereof. In an exemplary embodiment, the invention provides a formulation which comprises: (a) violaxanthin; (b) PTS; and (c) Vitamin C, a Vitamin C derivative, or combinations thereof.

In an exemplary embodiment, the invention provides a formulation which comprises: (a) asiatic acid; (b) a solubilizing agent (e.g., PTS); and (c) a stabilizer. In an exemplary embodiment, the invention provides a formulation which comprises: (a) asiatic acid; (b) a solubilizing agent; and (c) Vitamin C, a Vitamin C derivative, or combinations thereof. In an exemplary embodiment, the invention provides a formulation which comprises: (a) asiatic acid; (b) PTS; and (c) Vitamin C, a Vitamin C derivative, or combinations thereof.

In an exemplary embodiment, the invention provides a formulation which comprises: (a) β-sitosterol; (b) a solubilizing agent (e.g., PTS); and (c) a stabilizer. In an exemplary embodiment, the invention provides a formulation which comprises: (a) β-sitosterol; (b) a solubilizing agent; and (c) Vitamin C, a Vitamin C derivative, or combinations thereof. In an exemplary embodiment, the invention provides a formulation which comprises: (a) sitosterol; (b) PTS; and (c) Vitamin C, a Vitamin C derivative, or combinations thereof.

In an exemplary embodiment, the invention provides a formulation which comprises: (a) a tocopherol; (b) a solubilizing agent (e.g., PTS); and (c) a stabilizer. In an exemplary embodiment, the invention provides a formulation which comprises: (a) tocopherol; (b) a solubilizing agent; and (c) Vitamin C, a Vitamin C derivative, or combinations thereof. In an exemplary embodiment, the invention provides a formulation which comprises: (a) tocopherol; (b) PTS; and (c) Vitamin C, a Vitamin C derivative, or combinations thereof.

In an exemplary embodiment, the invention provides a formulation which comprises: (a) alpha-linolenic acid; (b) a solubilizing agent (e.g., PTS); and (c) a stabilizer. In an exemplary embodiment, the invention provides a formulation which comprises: (a) alpha-linolenic acid; (b) a solubilizing agent; and (c) Vitamin C, a Vitamin C derivative, or combinations thereof. In an exemplary embodiment, the invention provides a formulation which comprises: (a) alpha-linolenic acid; (b) PTS; and (c) Vitamin C, a Vitamin C derivative, or combinations thereof.

In an exemplary embodiment, the invention provides a formulation which comprises: (a) linoleic acid; (b) a solubilizing agent (e.g., PTS); and (c) a stabilizer. In an exemplary embodiment, the invention provides a formulation which comprises: (a) linoleic acid; (b) a solubilizing agent; and (c) Vitamin C, a Vitamin C derivative, or combinations thereof. In an exemplary embodiment, the invention provides a formulation which comprises: (a) linoleic acid; (b) PTS; and (c) Vitamin C, a Vitamin C derivative, or combinations thereof.

In an exemplary embodiment, the invention provides a formulation which comprises: (a) oleic acid; (b) a solubilizing agent (e.g., PTS); and (c) a stabilizer. In an exemplary embodiment, the invention provides a formulation which comprises: (a) oleic acid; (b) a solubilizing agent; and (c) Vitamin C, a Vitamin C derivative, or combinations thereof. In an exemplary embodiment, the invention provides a formulation which comprises: (a) oleic acid; (b) PTS; and (c) Vitamin C, a Vitamin C derivative, or combinations thereof.

In an exemplary embodiment, the invention provides a formulation which comprises: (a) gamma linolenic acid; (b) a solubilizing agent (e.g., PTS); and (c) a stabilizer. In an exemplary embodiment, the invention provides a formulation which comprises: (a) gamma linolenic acid; (b) a solubilizing agent; and (c) Vitamin C, a Vitamin C derivative, or combinations thereof. In an exemplary embodiment, the invention provides a formulation which comprises: (a) gamma linolenic acid; (b) PTS; and (c) Vitamin C, a Vitamin C derivative, or combinations thereof.

In an exemplary embodiment, the invention provides a formulation which comprises: (a) docosahexaenoic acid; (b) a solubilizing agent (e.g., PTS); and (c) a stabilizer. In an exemplary embodiment, the invention provides a formulation which comprises: (a) docosahexaenoic acid; (b) a solubilizing agent; and (c) Vitamin C, a Vitamin C derivative, or combinations thereof. In an exemplary embodiment, the invention provides a formulation which comprises: (a) docosahexaenoic acid; (b) PTS; and (c) Vitamin C, a Vitamin C derivative, or combinations thereof. In an exemplary embodiment, the invention includes from about 0.01% (w/w) to about 5% (w/w) of docosahexaenoic acid. In an exemplary embodiment, the invention includes from about 0.01% (w/w) to about 0.1% (w/w) of docosahexaenoic acid. In an exemplary embodiment, the invention includes from about 0.01% (w/w) to about 1% (w/w) of docosahexaenoic acid. In an exemplary embodiment, the invention includes from about 0.1% (w/w) to about 1% (w/w) of docosahexaenoic acid. In an exemplary embodiment, the invention includes from about 0.1% (w/w) to about 0.75% (w/w) of docosahexaenoic acid. In an exemplary embodiment, the invention includes from about 1% (w/w) to about 3% (w/w) of docosahexaenoic acid. In an exemplary embodiment, the invention includes from about 0.05% (w/w) to about 0.25% (w/w) of docosahexaenoic acid.

In an exemplary embodiment, the invention provides a formulation which comprises: (a) eicosapentaenoic acid; (b) a solubilizing agent (e.g., PTS); and (c) a stabilizer. In an exemplary embodiment, the invention provides a formulation which comprises: (a) eicosapentaenoic acid; (b) a solubilizing agent; and (c) Vitamin C, a Vitamin C derivative, or combinations thereof. In an exemplary embodiment, the invention provides a formulation which comprises: (a) eicosapentaenoic acid; (b) PTS; and (c) Vitamin C, a Vitamin C derivative, or combinations thereof. In an exemplary embodiment, the invention includes from about 0.01% (w/w) to about 5% (w/w) of eicosapentaenoic acid. In an exemplary embodiment, the invention includes from about 0.01% (w/w) to about 0.1% (w/w) of eicosapentaenoic acid. In an exemplary embodiment, the invention includes from about 0.01% (w/w) to about 1% (w/w) of eicosapentaenoic acid. In an exemplary embodiment, the invention includes from about 0.1% (w/w) to about 1% (w/w) of eicosapentaenoic acid. In an exemplary embodiment, the invention includes from about 0.1% (w/w) to about 0.75% (w/w) of eicosapentaenoic acid. In an exemplary embodiment, the invention includes from about 1% (w/w) to about 3% (w/w) of eicosapentaenoic acid. In an exemplary embodiment, the invention includes from about 0.05% (w/w) to about 0.25% (w/w) of eicosapentaenoic acid.

In an exemplary embodiment, the invention provides a formulation which comprises: (a) alpha-lipoic acid; (b) a solubilizing agent (e.g., PTS); and (c) a stabilizer. In an exemplary embodiment, the invention provides a formulation which comprises: (a) alpha lipoic acid; (b) a solubilizing agent; and (c) Vitamin C, a Vitamin C derivative, or combinations thereof. In an exemplary embodiment, the invention provides a formulation which comprises: (a) alpha lipoic acid; (b) PTS; and (c) Vitamin C, a Vitamin C derivative, or combinations thereof.

In an exemplary embodiment, the invention provides a formulation which comprises: (a) glutathione; (b) a solubilizing agent (e.g., PTS); and (c) a stabilizer. In an exemplary embodiment, the invention provides a formulation which comprises: (a) glutathione; (b) a solubilizing agent; and (c) Vitamin C, a Vitamin C derivative, or combinations thereof. In an exemplary embodiment, the invention provides a formulation which comprises: (a) glutathione; (b) PTS; and (c) Vitamin C, a Vitamin C derivative, or combinations thereof.

In an exemplary embodiment, the invention provides a formulation which comprises: (a) alpha-tocopherol; (b) a solubilizing agent (e.g., PTS); and (c) a stabilizer. In an exemplary embodiment, the invention provides a formulation which comprises: (a) alpha-tocopherol; (b) a solubilizing agent; and (c) Vitamin C, a Vitamin C derivative, or combinations thereof. In an exemplary embodiment, the invention provides a formulation which comprises: (a) alpha-tocopherol; (b) PTS; and (c) Vitamin C, a Vitamin C derivative, or combinations thereof.

In an exemplary embodiment, the invention provides a formulation which comprises: (a) alpha-tocotrienol; (b) a solubilizing agent (e.g., PTS); and (c) a stabilizer. In an exemplary embodiment, the invention provides a formulation which comprises: (a) alpha-tocotrienol; (b) a solubilizing agent; and (c) Vitamin C, a Vitamin C derivative, or combinations thereof. In an exemplary embodiment, the invention provides a formulation which comprises: (a) alpha-tocotrienol; (b) PTS; and (c) Vitamin C, a Vitamin C derivative, or combinations thereof.

In an exemplary embodiment, the invention provides a formulation which comprises: (a) a flavor oil (e.g., citrus flavor); (b) a solubilizing agent (e.g., PTS); and (c) a stabilizer. In an exemplary embodiment, the invention provides a formulation which comprises: (a) a flavor oil (e.g., citrus flavor); (b) a solubilizing agent; and (c) Vitamin C, a Vitamin C derivative, or combinations thereof. In an exemplary embodiment, the invention provides a formulation which comprises: (a) a flavor oil (e.g., citrus flavor); (b) PTS; and (c) Vitamin C, a Vitamin C derivative, or combinations thereof.

In an exemplary embodiment according to any of the above embodiments, the vitamin C derivative is not a lipid-soluble vitamin C derivative, such as ascorbyl palmitate. In another example according to any of the above embodiments, the stabilizer is not an ascorbyl moiety with a fatty acid counterion. In yet another example according to any of the above embodiments, the solubilizing agent is a member selected from PTS, PQS, PSS and PCS.

### V. Methods

### Methods of Making the Formulations

The invention also provides methods (e.g., processes) of making the formulations and compositions of the invention.

In an exemplary embodiment, the lipophilic bioactive molecule (e.g., ubiquinone and/or ubiquinol), solubilizing agent and reducing agent (e.g., vitamin C or a water-soluble vitamin C derivative) and optionally other components of the formulation are placed in a container. A solvent is then added and the mixture is optionally heated, thereby dissolving the components and forming the formulation.

In another exemplary embodiment, the lipophilic bioactive molecule (e.g., ubiquinone and/or ubiquinol) is dissolved in a solvent optionally using heat. The solubilizing agent, the reducing agent (e.g., vitamin C or a water-soluble vitamin C derivative) and optionally other components are added to the above solution creating a mixture, which is stirred and optionally heated to dissolve all components in the mixture, thus creating the formulation.

In another exemplary embodiment, a solubilizing agent is dissolved in a solvent (e.g., water). The lipophilic bioactive molecule (e.g., ubiquinone and/or ubiquinol) and the reducing agent (e.g., vitamin C or a water-soluble vitamin C derivative) together with any optional components are added and dissolved in the above solution (optionally using heat), thus creating the formulation.

In another exemplary embodiment, the reducing agent (e.g., vitamin C or a water-soluble vitamin C derivative) is dissolved in a solvent of choice. The lipophilic bioactive molecule (e.g., ubiquinone and/or ubiquinol) and the solubilizing agent together with any optional components are added and are dissolved in the solution (optionally using heat), thus creating the formulation.

In another exemplary embodiment, any optional components described herein are dissolved in a solvent of choice, and then the lipophilic bioactive molecule (e.g., ubiquinone, DHA, ALA), and a Vitamin C or a Vitamin C derivative and a solubilizing agent are dissolved in the solvent, thus creating the formulation.

In another exemplary embodiment, a lipophilic bioactive molecule (e.g., ubiquinone, DHA, ALA) and a solubilizing agent are dissolved in a solvent of choice, and then a Vitamin C or a Vitamin C derivative and any optional components are dissolved in the solvent, thus creating the formulation.

In another exemplary embodiment, the lipophilic bioactive molecule (e.g., ubiquinone, DHA, ALA) and a Vitamin C or a Vitamin C derivative are dissolved in a solvent of choice, and then a solubilizing agent and any optional components are dissolved in the solvent, thus creating the formulation.

In another exemplary embodiment, a solubilizing agent and a Vitamin C or a Vitamin C derivative are dissolved in a solvent of choice, and then the lipophilic bioactive molecule (e.g., ubiquinone, DHA, ALA) and any optional components are dissolved in the solvent, thus creating the formulation.

In another exemplary embodiment, the lipophilic bioactive molecule (e.g., ubiquinone, DHA, ALA), a solubilizing agent and a Vitamin C or a Vitamin C derivative are dissolved in a solvent of choice, and then any optional components are dissolved in the solvent, thus creating the formulation.

In another exemplary embodiment, a solubilizing agent, a Vitamin C or a Vitamin C derivative, and any optional components are dissolved in a solvent of choice, and then the lipophilic bioactive molecule (e.g., ubiquinone, DHA, ALA) is dissolved in the solvent, thus creating the formulation. In another exemplary embodiment, a solubilizing agent, a Vitamin C or a Vitamin C derivative, and any optional components are dissolved in a solvent of choice, and then an ubiquinone is dissolved in the solvent, thus creating the formulation.

In another exemplary embodiment, the lipophilic bioactive molecule (e.g., ubiquinone, DHA, ALA), a solubilizing agent, and any optional components are dissolved in a solvent of choice, and then a Vitamin C or a Vitamin C derivative is dissolved in the solvent, thus creating the formulation.

In another exemplary embodiment, the lipophilic bioactive molecule (e.g., ubiquinone, DHA, ALA), a Vitamin C or a Vitamin C derivative, and any optional components are dissolved in a solvent of choice, and then a solubilizing agent is dissolved in the solvent, thus creating the formulation.

### Exemplary Processes

### Method for Making a Water-Soluble Ubiquinol Stock Solution

In one aspect, the invention provides a method for making an aqueous, water-soluble ubiquinol formulation of the invention. An exemplary process for making an aqueous, water-soluble ubiquinol stock solution includes: contacting an emulsion of ubiquinone (e.g., CoQ₁₀) in an aqueous medium (e.g., water) (ubiquinone emulsion) with an amount of a water-soluble reducing agent (e.g., vitamin C or a water-soluble derivative of vitamin C) that is sufficient to essentially quantitatively reduce the ubiquinone to ubiquinol (e.g., ubiquinol-50). In one example, the aqueous ubiquinol formulation thus formed is essentially clear. In another example, the aqueous ubiquinol formulation thus formed becomes essentially clear upon dilution (e.g., 1:2, 1:4, 1:6, 1:8, 1:10, 1:20, 1:40, 1:60, 1:80 or 1:100 dilution) with water, the above aqueous medium or another aqueous solution (e.g., an original beverage).

The inventors have discovered that the above process, in which ubiquinol is formed in situ from solubilized (emulsified) ubiquinone is superior to a related process, in which pre-formed (isolated) ubiquinol (commercially available from, e.g., Kaneka) is contacted with a solubilizing agent and an aqueous medium. The aqueous formulation formed from isolated ubiquinol is typically not clear, and in certain examples cannot be converted to a clear solution even when heating. Another improvement of the current process stems from the fact that isolated ubiquinol is not chemically stable, e.g., oxidizes easily to ubiquinone, especially once the molecule is placed in an aqueous medium. In addition, for certain applications ubiquinol must be handled under an inert atmosphere (e.g., nitrogen, argon) to prevent oxidation. It is generally preferred in the art to avoid usage of inert gas for large-scale processes. Hence, the current process is has several advantages. First, the process starts with widely available ubiquinone, which is much more cost-effective than using isolated ubiquinol. Second, once the ubiquinol is formed in situ, the ubiquinol is stable to chemical oxidation in the aqueous solution. Third, the process does not rely on inert gas to produce a defined product. Overall, the current process is more cost-effective and does not require sophisticated equipment.

In one example according to any of the above embodiments, the process further includes: forming the above ubiquinone emulsion by contacting ubiquinone with a solution containing a solubilizing agent of the invention dissolved in an aqueous medium (e.g., water), thereby forming a mixture. The process can further include heating the mixture to a temperature sufficient to form the emulsion. In one example, the mixture is heated to a temperature between about 40° C. and about 200° C. In another example, the mixture is heated to a temperature between about 60° C. and about 140° C. In yet another example, the mixture is heated to a temperature between about 80° C. and about 120° C. In a further example, the mixture is heated to a temperature between about 80° C. and about 100° C. (e.g., about 90° C.).

In one example the amount of water-soluble reducing agent contacted with the ubiquinone emulsion is further sufficient to prevent re-oxidation of the ubiquinol to ubiquinone once the ubiquinol is formed. In one example, the reducing agent is contacted with the ubiquinone emulsion in an amount equivalent to a ratio of ubiquinol/ubiquinone to water soluble reducing agent of about 1:1 to about 1:10 (w/w). In another embodiment, the ratio of ubiquinol/ubiquinone to water soluble reducing agent is selected from about 1:1 to about 1:8 (w/w), from about 1:1 to about 1:6 (w/w) or from about 1:1 to about 1:4 (w/w). In yet another embodiment, the ratio of ubiquinol/ubiquinone to water soluble reducing agent is between about 1:1 to about 1:3 (w/w).

The ubiquinone in the above emulsion is solubilized in the aqueous medium using a solubilizing agent of the invention. In one example, the solubilizing agent used in the methods of the invention has a structure according to Formula (III) described herein. In another example, the solubilizing agent useful in the methods of the invention has a structure according to Formula (IV):

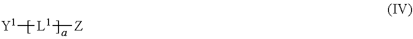

wherein the integer a, Y¹, L¹ and Z are defined as herein above. In a particular example, the solubilizing agent is selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof. In one embodiment, the solubilizing agent used in the methods of the invention is PTS.

In one example, the ubiquinone is solubilized in the above emulsion in the form of micelles that are formed between the ubiquinone and the solubilizing agent. In one example, the micelles have a median particle size of less than about 60 nm (e.g., between about 20 and about 30 nm).

In one example according to any of the above embodiments, the amount of ubiquinone contacted with the solubilizing agent is equivalent to a ratio of ubiquinol/ubiquinone to solubilizing agent of about 1:0.3 (w/w) to about 1:20 (w/w). In another example, the ratio of ubiquinol/ubiquinone to solubilizing agent is selected from about 1:1 (w/w) to about 1:20 (w/w). In yet another example, the ratio of ubiquinol/ubiquinone to solubilizing agent is selected from about 1:1 (w/w) to about 1:10 (w/w). In a further example, the ratio of ubiquinol/ubiquinone to solubilizing agent is selected from about 1:2 (w/w) to about 1:5 (w/w). In a further example, the ratio of ubiquinol/ubiquinone to solubilizing agent is selected from about 1:2 (w/w) to about 1:4 (w/w). In another example, the ratio of ubiquinol/ubiquinone to solubilizing agent is about 1:3 (w/w). In yet another example, the ratio of ubiquinol/ubiquinone to solubilizing agent is selected from about 1:0.3 (w/w) to about 1:1 (w/w). In a further exemplary embodiment, the ratio of ubiquinol/ubiquinone to solubilizing agent is selected from about 1:0.5 (w/w) to about 1:2 (w/w).

In one example, the invention provides a ubiquinol stock solution, which is prepared by a method according to any of the above embodiments.

In one example, the above water-soluble ubiquinol stock solution can be used to prepare a beverage of the invention. Hence, in one embodiment, the above method further includes contacting the water-soluble ubiquinol stock solution with an original beverage to form a ubiquinol beverage of the invention. Exemplary original beverages useful in the methods of the invention are disclosed herein.

In another example, the above water-soluble ubiquinol stock solution can be used to chemically stabilize other lipophilic bioactive molecules, such as those typically vulnerable to chemical degradation (e.g., oxidation). Hence, in another embodiment, the method further includes contacting the water-soluble ubiquinol stock solution with a lipophilic bioactive molecule forming an aqueous formulation of the lipohilic bioactive molecule. In one example, the lipophilic bioactive molecule is chemically stable in this formulation. For example, the lipophilic bioactive molecule is stable in the formulation for at least 90 days when stored at about 4° C.

Exemplary lipophilic bioactive molecules, which can be stabilized using any of the above methods include omega-3-fatty acids (e.g., docosahexaenoic acid (DHA), eicosapentaenoic acid (EPA) and alpha-linolenic acid (ALA)), omega-6-fatty acids, omega-9-fatty acids, carotenoids, essential oils, flavor oils and lipophilic vitamins. Exemplary carotenoids include lutein, astaxanthin, lycopene, fucoxanthin and canthaxanthin. Additional carotenoids (e.g., xanthophylls) are described herein.

### Method for Making an Aqueous Formulation of a Lipophilic Bioactive Molecule

In another aspect, the invention provides a method for making an aqueous formulation of a lipophilic bioactive molecule. In another aspect, the invention provides a method for chemically stabilizing a lipophilic bioactive molecule in an aqueous solution. An exemplary method includes: contacting an emulsion of the lipophilic bioactive molecule in an aqueous medium with an amount of a water-soluble reducing agent that is sufficient to prevent chemical degradation of the lipophilic bioactive molecule.

In one example, the amount of water-soluble reducing agent that is contacted with the above emulsion is equivalent to an over-stoichiometric mol ratio with respect to the lipophilic bioactive molecule. In another example, the amount is equivalent to a ratio of lipophilic bioactive molecule to water-soluble reducing agent of about 1:1 to about 1:10 (w/w). In yet another embodiment, the amount is equivalent to a ratio of lipophilic bioactive molecule to water-soluble reducing agent of about 1:1 to about 1:8 (w/w), about 1:1 to about 1:6 (w/w) or about 1:1 to about 1:4 (w/w). In yet another embodiment, the amount of water-soluble reducing agent used in the methods of the invention is equivalent to a ratio of lipophilic bioactive molecule to water-soluble reducing agent of about 1:1 to about 1:3 (w/w).

In one example according to any of the above embodiments, the lipophilic bioactive molecule in the above emulsion is solubilized in the aqueous medium using a solubilizing agent of the invention. In one example, the solubilizing agent used in the methods of the invention has a structure according to Formula (III) described herein. In another example, the solubilizing agent useful in the methods of the invention has a structure according to Formula (IV):

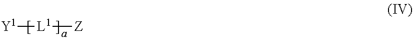

wherein the integer a, Y¹, L¹ and Z are defined as herein above. In a particular example, the solubilizing agent in the above method is selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof. In one embodiment, the solubilizing agent used in the methods of the invention is PTS.

In one example, the lipophilic bioactive molecule is solubilized in the above emulsion in the form of micelles that are formed between the lipophilic bioactive molecule and the solubilizing agent. In one example, the micelles have a median particle size of less than about 60 nm (e.g., between about 20 and about 30 nm).

In one example, the lipophilic bioactive molecule is chemically stable in the above water-soluble formulation. For example, the lipophilic bioactive molecule is stable in the formulation for at least 90 days when stored at about 4° C.

Exemplary lipophilic bioactive molecules, which can be stabilized according to any of the above embodiments include omega-3-fatty acids (e.g., docosahexaenoic acid (DHA), eicosapentaenoic acid (EPA) and alpha-linolenic acid (ALA)), omega-6-fatty acids, omega-9-fatty acids, carotenoids, essential oils, flavor oils and lipophilic vitamins. Exemplary carotenoids include lutein, astaxanthin, lycopene, fucoxanthin and canthaxanthin. Additional carotenoids (e.g., xanthophylls) are described herein.

### Methods of Making the Beverages

### Method for Making a Ubiquinol Beverage

In another aspect, the invention provides a method for making a beverage (e.g., a non-alcoholic beverage) that includes ubiquinol. An exemplary method includes: contacting an original beverage with a water-soluble ubiquinol stock solution (e.g., ubiquinol-50 stock solution) of the invention. Exemplary original beverages are disclosed herein and include carbonated or uncarbonated water, flavored water, soft drinks, beer and drinkable dairy products. All embodiments described herein above for the method of making a ubiquinol stock solution equally apply to the method of making a ubiquinol beverage described in this paragraph.

### Method for Making a Ubiquinone Beverage

In another aspect, the invention provides a method for making a beverage (e.g., a non-alcoholic beverage) that includes ubiquinone (e.g., CoQ₁₀). An exemplary method includes contacting an emulsion of ubiquinone in an aqueous medium (e.g., water) (ubiquinone emulsion) with an original beverage. The emulsion includes a solubilizing agent of the invention (e.g., of Formula (III) or Formula (IV)). The method can further include forming the ubiquinone emulsion, e.g., by contacting the ubiquinone with a solution of a solubilizing agent of the invention in anaqueous medium (e.g., water). Exemplary original beverages are disclosed herein and include carbonated or uncarbonated water, flavored water, soft drinks, beer and drinkable dairy products.

In one example according to any of the above embodiments, the solubilizing agent used in the methods of the invention has a structure according to Formula (III) described herein. In another example, the solubilizing agent useful in the methods of the invention has a structure according to Formula (IV):

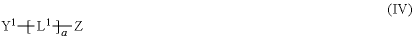

wherein the integer a, Y¹, L¹ and Z are defined as herein above. In a particular example, the solubilizing agent is selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof. In one embodiment, the solubilizing agent used in the methods of the invention is PTS.

In one example, the beverage is essentially clear.

In another example, the ubiquinone is solubilized in the above emulsion in the form of micelles that are formed between the ubiquinone and the solubilizing agent. In one example, the micelles have a median particle size of less than about 60 nm (e.g., between about 20 and about 30 nm).

In one example, the method further includes adding a vitamin (e.g., vitamin C, vitamin E, a B-vitamin (e.g., vitamin B-pentapalmitate) or combinations thereof) to the ubiquinone beverage. In one example, when the vitamin (e.g., vitamin E) is added to the beverage, the vitamin is first solubilized in an aqueous medium using a solubilizing agent, such as a solubilizing agent of the invention, and is subsequently added to the beverage. Exemplary solubilizing agents that can be used to solubilize the vitamin (e.g., vitamin E) include PTS, PSS, PCS, PQS and polyoxyethylene sorbitan monooleate.

In an examplary embodiment, the ubiquinone beverage can be oxygenated. The oxygenation can take place in a manner described in detail in WO 95/32796 (e.g., to an oxygen (O₂) content of about 20, 40, 60, 80 or 100 mg/l. Oxygenation of the beverage can resulted in a clear beverage.

In another embodiment, the invention provides a beverage produced by any of the above methods of the invention.

### Methods of Stabilizing Lipophilic Bioactive Molecules in Aqueous Solutions

### Stabilizing a Lipophilic Bioactive Molecule Using Ubiquinol/Vitamin C

In one aspect the invention provides a method of stabilizing a lipophilic bioactive molecule in an aqueous formulation. An exemplary method includes contacting the lipophilic bioactive molecule with a ubiquinol stock solution of the invention. Exemplary ubiquinol stock solutions (e.g., ubiquinol-50 stock solutions) and methods of making ubiquinol stock solutions are disclosed herein. In one example, the method further includes making the ubiquinol stock solution, for example, by first solubilizing ubiquinone in an aqueous medium (e.g., water) using a solubilizing agent of the invention, and then contacting the ubiquinone solution with a water-soluble reducing agent of the invention, thereby reducing ubiquinone to ubiquinol.

### Stabilizing a Lipophilic Bioactive Molecule Using a Water-Soluble Reducing Agent

In another aspect the invention provides a method of stabilizing a lipophilic bioactive molecule in an aqueous solution using a water-soluble reducing agent of the invention. An exemplary method includes contacting an emulsion of a lipophilic bioactive molecule in an aqueous medium with an amount of a water-soluble reducing agent sufficient to prevent chemical degradation of the lipophilic bioactive molecule. The emulsion includes a solubilizing agent of the invention. In one example, the solubilizing agent used in the methods of the invention has a structure according to Formula (III) described herein. In another example, the solubilizing agent useful in the methods of the invention has a structure according to Formula (IV):

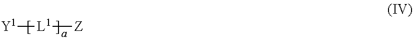

wherein the integer a, Y¹, L¹ and Z are defined as herein above. In a particular example, the solubilizing agent is selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof. In one embodiment, the solubilizing agent used in the methods of the invention is PTS.

In one example according to any of the above embodiments, the ratio of the lipophilic bioactive molecule to the water-soluble reducing agent is between about 100:1 (w/w) and about 1:10 (w/w). In another example, the ratio of the lipophilic bioactive molecule to the water-soluble reducing agent is between about 10:1 (w/w) and about 1:10 (w/w).

In another example according to any of the above embodiments, the lipophilic bioactive molecule is essentially stable with respect to chemical degradation for at least 90 days when the formulation is stored at about 4° C.

In yet another example according to any of the above embodiments, the bioactive, lipophilic molecule is a member selected from omega-3-fatty acids, omega-6-fatty acid, carotenoids, essential oils, flavor oils and lipophilic vitamins. In one example, the omega-3-fatty acid is a member selected from docosahexaenoic acid (DHA), eicosapentaenoic acid (EPA) and alpha-linolenic acid (ALA). In another example, the carotenoid is a member selected from lutein, astaxanthin, lycopene, fucoxanthin and canthaxanthin.

Any of the embodiments outlined herein above for the compositions of the invention (section III. of the application) equally apply to any of the methods of the invention (section V. of the application), e.g., to any of the above embodiments.

The compositions and methods of the present invention are further illustrated by the examples that follow. These examples are offered to illustrate, but not to limit the claimed invention.

### EXAMPLES

The following abbreviations are used throughout the Examples:

CoQ₁₀—coenzyme Q₁₀
Ub50—ubiquinol-50
PQS—polyoxyethanyl-ubiquinol-sebacate
PTS—polyoxyethanyl-tocopherol-sebacate
PSS—polyoxyethanyl-sitosteryl-sebacate

A number following one of the above abbreviations (e.g., PQS-600) indicates an average molecular weight of the polyoxyethanyl or poly(ethylene glycol) (PEG) moiety of the compound. A number followed by the abbreviation “Me” (e.g., PQS-750Me) indicates a polyoxyethanyl moiety capped with a methyl group (methoxypolyoxyethanyl or mPEG).

### Example 1

### Non-Aqueous Ubiquinol Formulations

This example sets forth non-aqueous (e.g., water-soluble) formulations of ubiquinol and methods for making the formulations. In an exemplary method, ubiquinone (e.g., CoQ₁₀) is dissolved in a polyhydric alcohol, an oil or another carrier and is subsequently reduced to ubiquinol (e.g., ubiquinol-50) utilizing a water-soluble reducing agent (e.g., vitamin C). The reaction can be described by the following scheme:

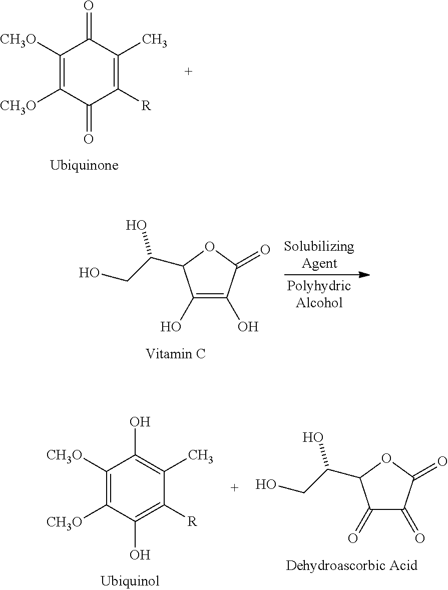

In the above scheme, the solubilizing agent can be, for example, PTS, PCS, PSS, PQS or other surfactants, such as Tween 80 optionally in combination with an oil.

In these formulations, the reduced ubiquinol is optionally stabilized by an excess of water-soluble reducing agent (e.g., vitamin C). In one example, the resulting formulation was added to a soft gelatin capsule. In another example, the formulation can be used as a stabilizer (i.e., to prevent chemical degradation) in nutraceutical formulations or food preparations. In one example, the formulation was used to stabilize carotenoids. In other examples, the formulations can be used in skin-care products.

### 1.1. Ubiquinol Formulations Including a Polyhydric Alcohol

General Procedure 1: In a jacketed mixing vessel, glycerine, propylene glycol and/or another polyhydric alcohol (60%-99%) were mixed with PTS (1% to 40%). The mixture was heated to about 55° C. (+/−5° C.) while mixing constantly. CoQ₁₀ (e.g., 0.5% to about 10% w/w) was then added while stirring and the mixture was stirred at about 55° C. for 1-2 hours. Vitamin C (or a suitable water-soluble vitamin C derivative) was added in an amount ranging from about 1% to about 15% w/w and the mixture was again stirred at elevated temperature for about 1 to 2 hours or at least until the mixture was clear and almost colorless (indicating that the reduction of ubiquinone to ubiquinol was complete). The mixing vessel was then connected to a cooling system and the mixture was slowly cooled to room temperature (e.g., about 23° C.+−3° C.) while mixing continuously. The cooled liquid was transferred to a stainless steel drum, which was then flushed with nitrogen and sealed. The product was analyzed by HPLC to quantitatively determine the ubiquinone and ubiquinol content. In one example, ubiquinone was either not detectable or the ratio ubiquinone:ubiquinol was below about 5%. The liquid was optionally encapsulated in soft-gelatin capsules (e.g., utilizing standard manufacturing procedures). The soft-gelatin material optionally included an opacifier (TiO₂) and/or a colorant.

An exemplary composition prepared by the above described method has the following components (w/w; excluding gelatin capsule):
Propylene Glycol 80%
PTS 10%
Ubiquinol/CoQ₁₀ 5%
Vitamin C 5%

### 1.2. Ubiquinol Formulations Including Triglycerides

The following components were mixed in a jacketed mixing vessel: PTS (2-20% w/w), hydroxylated lecithin (2%-20% w/w), phosphatidyl chloline solution (20% to 50% w/w), medium chain triglycerides, other suitable vegetable oil (5% to 40% w/w) and a surface-active excipient (e.g., Gelucire) (5% to 50% w/w). The mixture was heated to about 55° C. (+/−5° C.) while mixing constantly. Coenzyme Q (0.5% to about 10% w/w) was added while stirring and the mixture was stirred at about 55° C. for a period of about 1 hour. Vitamin C (or a suitable water-soluble vitamin C derivative) was then added (1% to about 10% w/w) and the mixture was again stirred at elevated temperature for 1 to 2 hours or at least until the mixture was clear and essentially colorless (indicating that the reduction of ubiquinone to ubiquinol was complete). The crude product was further processed as described in Example 1. The product was analyzed using HPLC. In one example, ubiquinone was either not detectable or the ratio of ubiquinone:ubiquinol was below about 5%.

An exemplary composition prepared by the above described method has the following components (w/w; excluding gelatin capsule):
PTS 6%
Hydroxylated Lecithin 4%
Phosphatidyl Choline Solution (52%) 32%
MCT (medium chain triglycerides) 20%
Gelucire 30%
Ubiquinol/CoQ₁₀, 4%
Vitamin C 4%

### Example 2

### Aqueous Ubiquinone and Ubiquinol Formulations

This example sets forth aqueous formulations of ubiquinol and methods for making the formulations. In an exemplary method, ubiquinone (e.g., CoQ₁₀) is dissolved in a PTS/water mixture forming a water-soluble formulation of ubiquinone. Ubiquinone in this water-soluble form, can be reduced (e.g., quantitatively) to ubiquinol (e.g., ubiquinol-50) utilizing a water-soluble reducing agent, such as vitamin C. For example, the water-soluble form of ubiquinone and vitamin C undergo a reduction/oxidation reaction as follows:

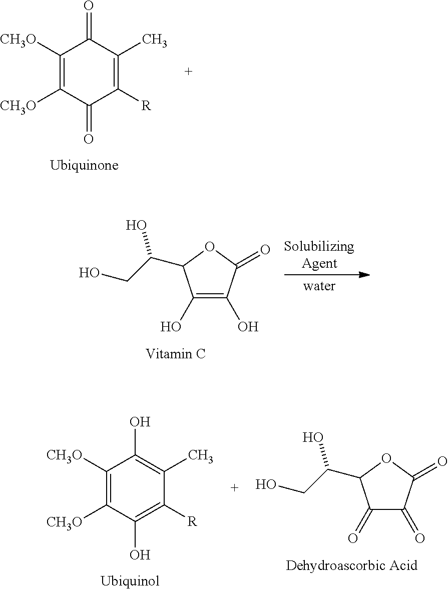

In the above scheme, the solubilizing agent can be, for example, PTS, PCS, PSS, PQS or other surfactants, such as Tween 80 optionally in combination with an oil.

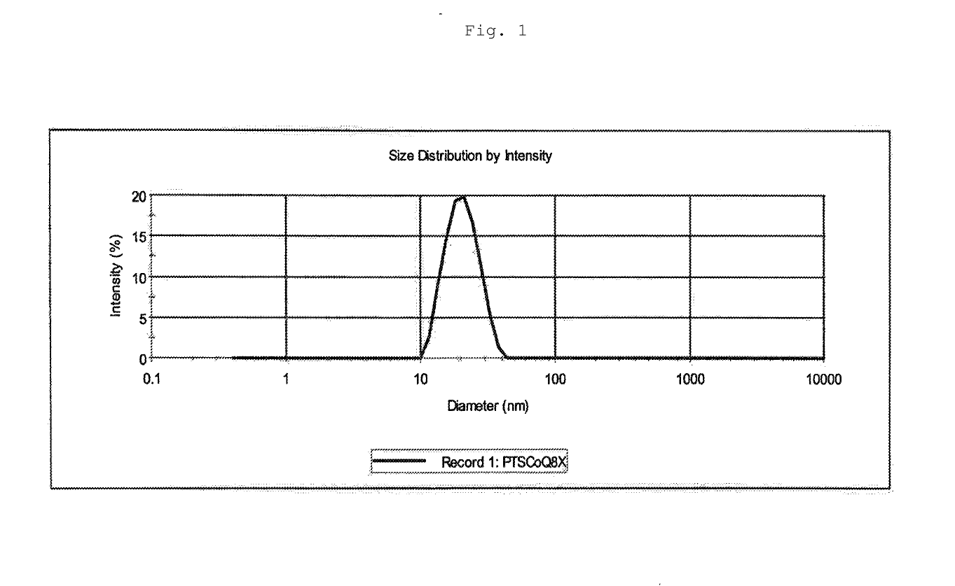

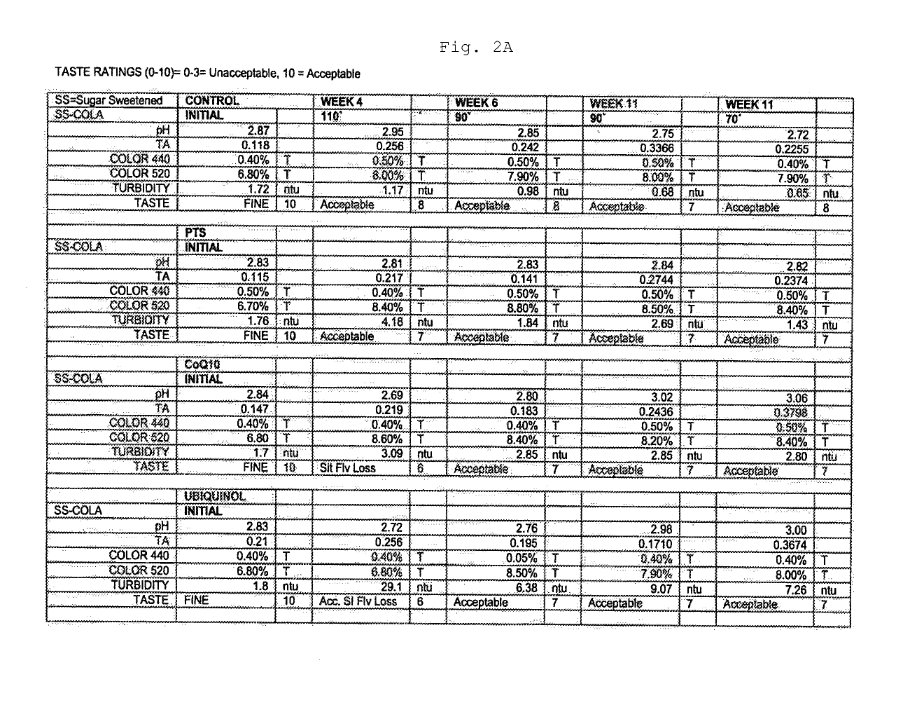

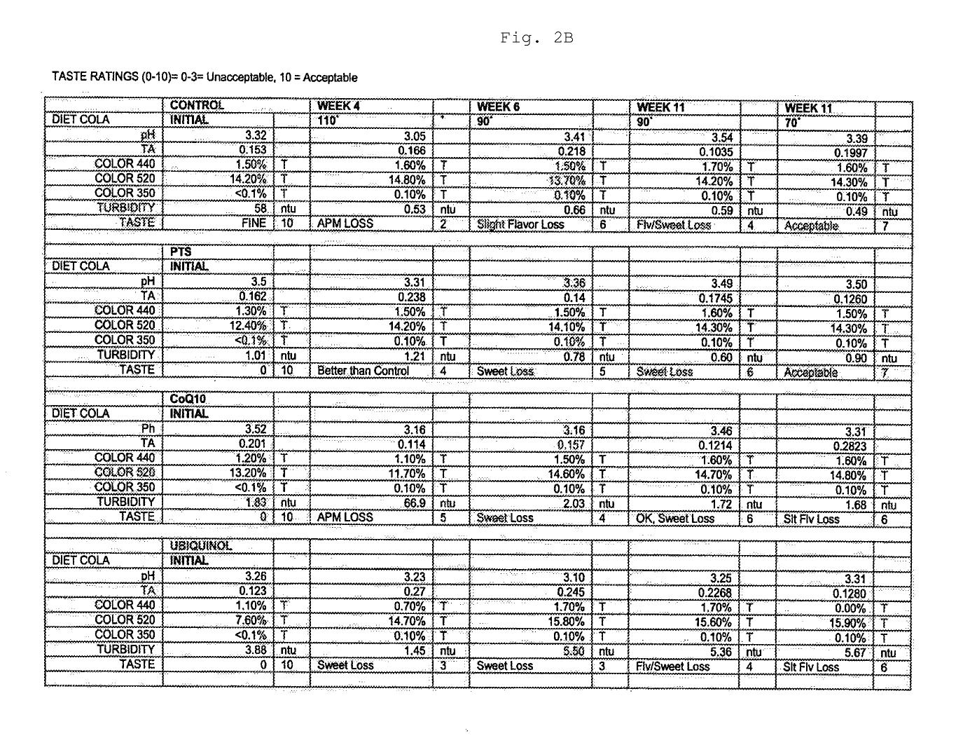

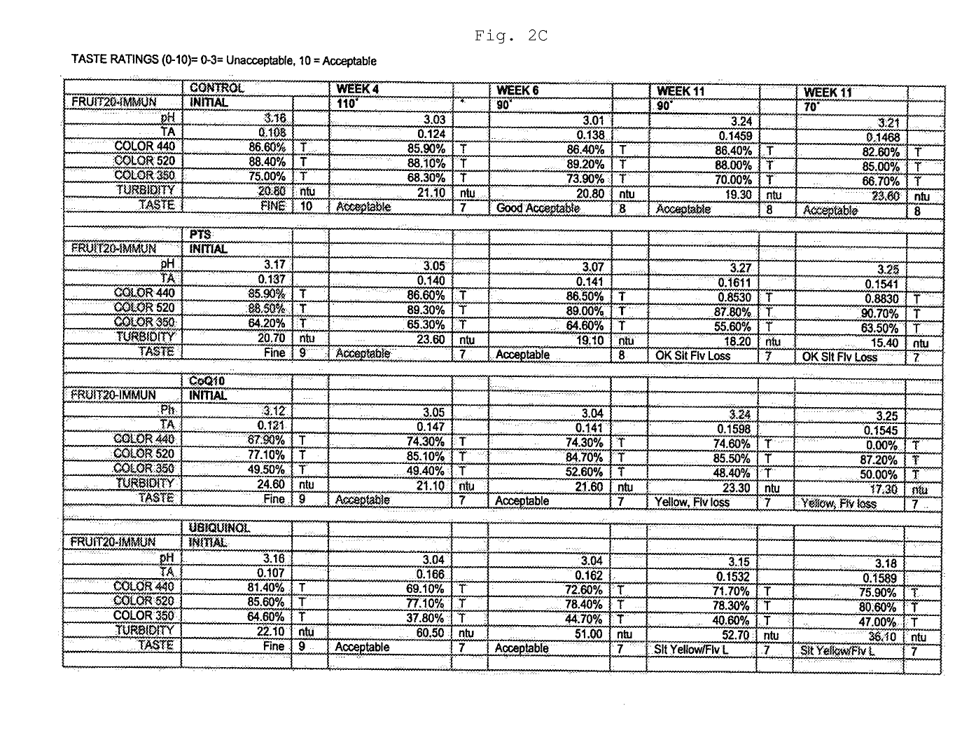

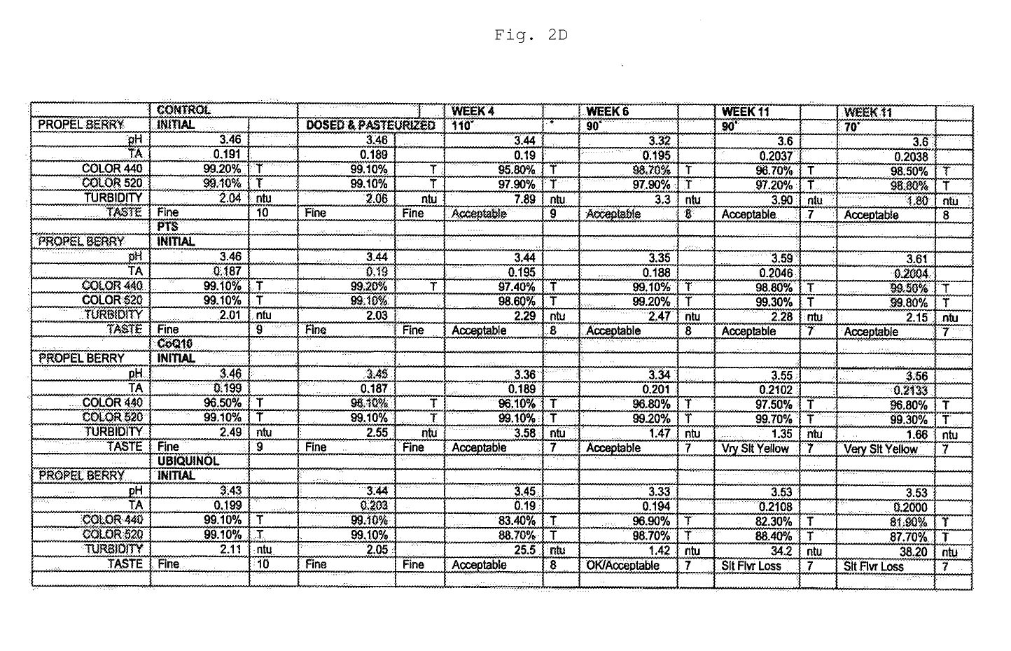

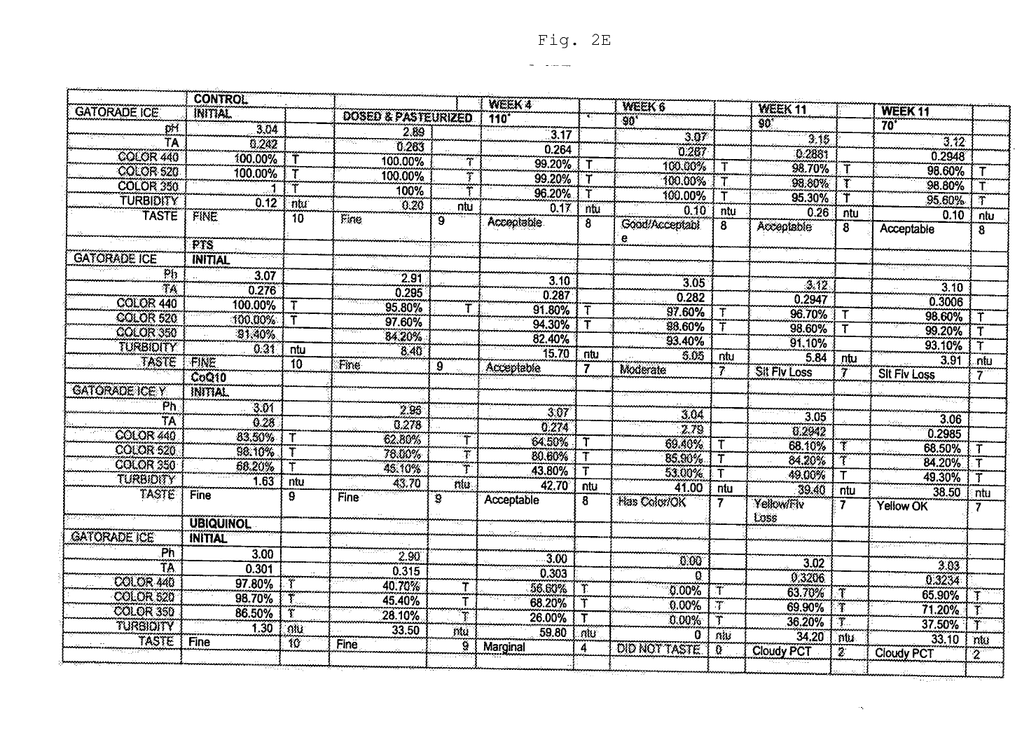

General Procedure 2: In a 50-L jacketed vessel under argon were combined a solubilizing agent (e.g., PTS-600, 3.00 kg) and water (8.5 L). The mixture was heated to a temperature between about 60 and about 90° C. and was stirred at 60 to 65° C. for about 30 min until the mixture was homogenous. Under stirring, the lipophilic bioactive molecule (e.g., CoQ₁₀, 1.0 kg) was added and the stirred mixture was heated to 90-95° C. for about 1 hour until the mixture was an emulsion. It was then slowly cooled to 10-15° C. at a rate of about 10° C. per hour using a temperature controller. The cooled mixture was typically clear. The ubiquinone emulsion was analyzed by DLS to determine the median particle size of the micelles formed between CoQ₁₀ and the solubilizing agent. An exemplary result of this analysis is shown in FIG. 1. The median particle size of CoQ₁₀ in water using PTS is between about 20 and 30 nm. This ubiquinone emulsion can optionally be sterile-filtered (0.2 μm filter) and utilized as an additive for foods and beverages or consumer products, such as skin-care products.

In one example, the above ubiquinone formulation was further processed by addition of an excess (over-stoichiometric amount—with respect to the amount of ubiquinone, e.g., 3 kg) of vitamin C to reduce the ubiquinone to ubiquinol. The mixture was stirred until the reduction was complete (e.g., 24-72 hours). The emulsion was optionally sterile-filtered and stored. In one example, the ubiquinol formulation is stored at about 4-10° C., optionally under argon and is typically stable with respect to ubiquinol degradation (e.g., oxidation to ubiquinol) for at least 3 month.

The described water-soluble ubiquinol formulation can be used, for example, as an additive for foods and beverages as described herein. The formulations can also be used in consumer products, such as cosmetics and other skin-care products (i.e., topical application). An important advantage of these ubiquinol formulations is that they can be used without further processing (i.e., isolation and purification of the active component).

### Example 3

### Water-Soluble Formulations of Other Lipophilic Molecules

This example sets forth aqueous formulations of lipophilic bioactive molecules, which are prepared using a solubilizing agent of the invention.

General Procedure 3A: In a jacketed mixing vessel were combined water (60%-99% w/w) and PTS (1% to 40% w/w) and the mixture was heated to about 55° C. (+/−5° C.) while mixing constantly. Under stirring, the lipophilic, bioactive molecule (e.g., 0.5% to about 10% w/w) was then added and the mixture was stirred at the elevated temperature for a period of 1-2 hours until the mixture was homogenous. The mixing vessel was then connected to a cooling system and the mixture was slowly cooled to room temperature (about 23° C.+/−3° C.) while stirring. The liquid was transferred to a stainless steel drum, which was then flushed with nitrogen and sealed. The product was analyzed using HPLC for quantitative determination of the lipophilic bioactive molecule.

General Procedure 3B: In a jacketed mixing vessel were combined PTS (2.00 kg) and the lipophilic, bioactive molecule (e.g., DHA-S, 1.00 kg). The mixture was stirred at 40-45° C. (+/−5° C.) for about 30 min until homogenous. To the mixture was added water (7.00 kg) and the aqueous mixture was stirred at 70-75° C. for about 1 hour until an emulsion was formed. The emulsion was then slowly cooled to 10-15° C. at a rate of about 10° C. per hour using a temperature controller. The cooled mixture was typically clear. The emulsion was optionally sterile-filtered (e.g., 0.2 μm filter).

Lipophilic bioactive molecules that were formulated using General Procedures 3A and 3B include DHA, lutein and astaxanthin. Exemplary formulations of these molecules are described below:

### 3.1. Exemplary Formulation of Omega-3-Fatty Acid

| | |
|---|---|
| DHA | 1 g (e.g., DHA-S or DHA-HM Oil, |
| | Colon Martek Biosciences) |
| PTS | 2 g |
| Water | 7 g |

### 3.2. Exemplary Formulation of Lutein

| | |
|---|---|
| Lutein | 0.010 g |
| PTS | 0.045 g |
| Water | 9.945 g |

### 3.3. Exemplary Formulation of Astaxanthin

| | |
|---|---|
| Zanthin Astaxanthin complex: | 0.05 g |
| 10% oleoresin | (equivalent of 0.005 g of Astaxanthin) |
| (Valensa International) | |
| PTS | 0.32 g |
| Coenzyme Q10 | 0.10 g |
| Water | 9.53 g |

### 3.4. Exemplary Formulation of Astaxanthin

| | |
|---|---|
| Astareal L10 | 0.30 g (equivalent of 0.027 g of Astaxanthin) |
| (Fuji Chemical) | |
| PTS | 0.90 g |

### 3.5. Exemplary Formulation of Astaxanthin

| | |
|---|---|
| PTS/AStreal L10 stock | 0.010 g (equivalent of 0.0002 g of astaxanthin) |
| (3:1 w/w) | |
| Water | 1.000 g |

### 3.6. Exemplary Formulation of Astaxanthin

| | |
|---|---|
| PTS/AStreal L10 stock | 0.005 g (equivalent of 0.0002 g of astaxanthin) |
| (1:1 w/w) | |
| Water | 1.000 g |

### Example 4

### Formulations of Lipophilic Bioactive Molecules Stabilized with Vitamin C

This example sets forth aqueous formulations of lipophilic bioactive molecules, which are prepared using a solubilizing agent of the invention and a water-soluble reducing agent.

General Procedure 4A: In a jacketed mixing vessel were combined water (60%-99% w/w) and PTS (1% to 40% w/w) and the mixture was heated to about 55° C. (+/−5° C.) while mixing constantly. Under stirring, the lipophilic, bioactive molecule (e.g., 0.5% to about 10% w/w) was added and the mixture was stirred at the elevated temperature for a period of 1-2 hours. To the mixture was then added vitamin C in an effective amount ranging from about 1% to about 15% w/w (see examples below) and the mixture was stirred at an elevated temperature for 1 to 2 hours or at least until the mixture was clear. The mixing vessel was then connected to a cooling system and the mixture was slowly cooled to room temperature (about 23° C.+/−3° C.) while stirring. The liquid was transferred to a stainless steel drum, which was then flushed with nitrogen and sealed. The product was analyzed using HPLC for quantitative determination of the lipophilic bioactive molecule.

4.1. Exemplary Formulation of an Omega-3-Fatty Acid with Vitamin C

| | |
|---|---|
| DHA | 1 g (e.g., DHA-S or DHA-HM Oil, |
| | Martek Biosciences) |
| PTS | 2 g |
| Water | 7 g |
| Vitamin C | 0.015 g (0.0015 g/mL) |

4.2. Exemplary Formulation of Lutein with Vitamin C

| | |
|---|---|
| Lutein | 0.010 g |
| PTS | 0.045 g |
| Water | 9.945 g |
| Vitamin C | 0.015 g (0.0015 g/ml) |

The above aqueous formulation of lutein was stable with respect to chemical degradation for at least 4 months when stored at about 4 to about 23° C.

4.3. Exemplary Formulation of Asthaxanthin with Vitamin C

A water-soluble stock solution of Astaxanthin was prepared according to General Procedure 3A or 3B:

| | |
|---|---|
| Zanthin Astaxanthin complex: | 0.05 g |
| 10% oleoresin (Valensa International) | (equivalent of 0.005 g of |
| | Astaxanthin) |
| PTS | 0.32 g |
| Coenzyme Q10 | 0.10 g |
| Water | 9.53 g |

The above water-soluble asthaxanthin stock solution was then diluted with water to prepare formulations with astaxanthin concentration of about 0.0002 g/ml and was stabilized with ascorbic acid 0.0015 g/ml. These aqueous formulations of asthaxanthin were stable with respect to chemical degradation for at least 5 months when when stored at about 4 to about 23° C.

In the following examples, astaxanthin and omega-3-fatty acids were stabilized in an aqueous formulation using a water-soluble PTS/ubiquinol stock solution of the invention prepared according to General Procedure 2 followed by reduction of ubiquinone with vitamin C (PTS 6 g; ubiquinol 2 g; ascorbic acid 6 g; water 21 g).

4.4. Exemplary Formulation of Asthaxanthin with Ubiquinol/Vitamin C

| | |
|---|---|
| Astareal L10 (Fuji Chemical) | 0.30 g (equivalent of 0.027 g of |
| | astaxanthin) |
| PTS | 0.90 g |
| Ubiquinol/vitamin C | 0.05 g (equivalent of 0.003 g ubiquinol; |
| composition | and 0.009 g of ascorbic acid) |

4.5. Exemplary Formulation of Asthaxanthin with Ubiquinol/Vitamin C

| | |
|---|---|
| PTS/AStreal L10 stock (3:1 w/w) | 0.010 g (equivalent of 0.0002 g |
| | of Astaxanthin) |
| Water | 1.000 g |
| Ubiquinol/vitamin C composition | 0.005 g (0.0003 g/ml ubiquinol and |
| | 0.0009 g/ml ascorbic acid) |

4.6. Exemplary Formulation of Asthaxanthin with Ubiquinol/Vitamin C

| | |
|---|---|
| AStreal L10/PTS stock (1:1 w/w) | 0.005 g (equivalent of 0.0002 g |
| | of Astaxanthin) |
| Water | 1.000 g |
| Ubiquinol/vitamin C composition | 0.005 g (0.0003 g/ml ubiquinol and |
| | 0.0009 g/ml ascorbic acid) |

4.7. Exemplary Formulation of an Omega-3-Fatty Acid with Ubiquinol/Vitamin C

| | |
|---|---|
| DHA | 1 g (e.g., DHA-S or DHA-HM Oil, |
| | Martek Biosciences) |
| PTS | 2 g |
| Water | 7 g |
| Ubiquinol/ascorbic | 0.072 g (equivalent to 0.015 g |
| acid composition | ascorbic acid) |

The above water-soluble asthaxanthin and DHA formulations of Examples 4.4. to 4.7. were stable with respect to chemical degradation for at least 6 months when stored at about 4° C.

### Example 5

### Preparation of Solubilizing Agents

General Procedure 5: To a solution of 0.83 g of 3-sitosterol (about 60%) in 3 mL dry toluene at 40° C. were added 1.33 mmole of triethylamine (TEA). To the stirred solution was added dropwise a solution of 1.33 mmole of sebacoyl chloride in 2 mL dry toluene under anhydrous conditions. The reaction mixture was stirred for about 10 min at room temperature. A solution of 2 mmole PEG-600 and 2.66 mmole TEA in 3 mL dry toluene was then added dropwise to the reaction mixture. The reaction mixture was stirring for an additional 20 min at room temperature and was then extracted with brine (4×3 mL). The toluene was removed under reduced pressure leaving a waxy residue. This crude product was dissolved in 15 mL water and water-insoluble particles were removed by filtration. The filtrate was lyophilized, yielding 0.8 g of polyoxyethanyl-sitosteryl sebacate as a pale-yellow waxy product (PSS-600).

Polyoxyethanyl-cholesteryl sebacate (PCS-600) was prepared from cholesterol and polyoxyethanyl-a-tocopheryl sebacate (PTS-600) was prepared from α-tocopherol according to General Procedure 5.

A person of ordinary skill in the art will appreciate that other solubilizing agents can be obtained using General Procedure 5 or modified versions thereof by linking any polyethylene glycol molecule (e.g., PEG having an average molecular weight higher than 600, e.g., 1000) or methoxy-polyethylene glycols (e.g., average molecular weight 750) to a sterol (e.g., cholesterol), a tocopherol or tocotrienol (e.g., α-tocopherol) or a ubiquinol (e.g., ubiquinol-50), using suitable coupling reagents, such as adipoyl, suberoyl, azelaoyl or dodecanedioyl dichlorides.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1.-81. (canceled)

82. A method for chemically stabilizing an omega-fatty acid in an aqueous formulation, said method comprising contacting an emulsion of said omega-fatty acid in an aqueous medium with an amount of a water-soluble reducing agent sufficient to prevent chemical degradation of said omega-fatty acid, wherein said emulsion comprises a solubilizing agent having a formula according to Formula (IV): 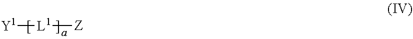
wherein
a is an integer selected from 0 and 1;
Z is a member selected from a sterol, a tocopherol, and derivatives or homologues thereof;
Y¹ is a linear or branched hydrophilic moiety comprising at least one polymeric moiety, wherein each of said polymeric moiety is a member independently selected from poly(alkylene oxides) and polyalcohols; and
L¹ is a linker moiety selected from substituted or unsubstituted alkyl, substituted or unsubstituted heteroalkyl, substituted or unsubstituted aryl, substituted or unsubstituted heteroaryl and substituted or unsubstituted heterocycloalkyl.

83. The method of claim 82, wherein the ratio of said omega-fatty acid to said water-soluble reducing agent is between about 100:1 (w/w) and about 1:10 (w/w).

84. The method of claim 82, wherein the ratio of said omega-fatty acid to said water-soluble reducing agent is between about 10:1 (w/w) and about 1:10 (w/w).

85. The method of claim 82, wherein said omega-fatty acid is stable with respect to chemical degradation for at least 90 days when said formulation is stored at about 4° C.

86. The method of claim 82, wherein said omega-fatty acid is an omega-3-fatty acid.

87. The method of claim 86, wherein said omega-3-fatty acid is a member selected from docosahexaenoic acid (DHA), eicosapentaenoic acid (EPA) and alpha-linolenic acid (ALA).

88.-89. (canceled)

90. The method of claim 82, wherein said water-soluble reducing agent is vitamin C, a vitamin C derivative, or a combination thereof.

91. The method of claim 82, wherein said solubilizing agent is a member selected from polyoxyethanyl-tocopheryl-sebacate (PTS), polyoxyethanyl-sitosterol-sebacate (PSS), polyoxyethanyl-cholesterol-sebacate (PCS), and combinations thereof.

92. The method of claim 82, wherein said omega-fatty acid is an omega-3-fatty acid; said water-soluble reducing agent is vitamin C; and said solubilizing agent is PTS.

93. A composition for chemically stabilizing an omega-fatty acid in an aqueous formulation, comprising an emulsion of said omega-fatty acid in an aqueous medium with a water-soluble reducing agent and a solubilizing agent having a formula according to Formula (IV): 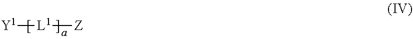
wherein
a is an integer selected from 0 and 1;
Z is a member selected from a sterol, a tocopherol, and derivatives or homologues thereof;
Y¹ is a linear or branched hydrophilic moiety comprising at least one polymeric moiety, wherein each of said polymeric moiety is a member independently selected from poly(alkylene oxides) and polyalcohols; and
L¹ is a linker moiety selected from substituted or unsubstituted alkyl, substituted or unsubstituted heteroalkyl, substituted or unsubstituted aryl, substituted or unsubstituted heteroaryl and substituted or unsubstituted heterocycloalkyl.

94. The composition of claim 93, wherein the ratio of said omega-fatty acid to said water-soluble reducing agent is between about 100:1 (w/w) and about 1:10 (w/w).

95. The composition of claim 93, wherein the ratio of said omega-fatty acid to said water-soluble reducing agent is between about 10:1 (w/w) and about 1:10 (w/w).

96. The composition of claim 93, wherein said omega-fatty acid is stable with respect to chemical degradation for at least 90 days when said formulation is stored at about 4° C.

97. The composition of claim 93, wherein said omega-fatty acid is an omega-3-fatty acid.

98. The composition of claim 97, wherein said omega-3-fatty acid is a member selected from docosahexaenoic acid (DHA), eicosapentaenoic acid (EPA) and alpha-linolenic acid (ALA).

99. The composition of claim 93, wherein said water-soluble reducing agent is vitamin C, a vitamin C derivative, or a combination thereof.

100. The composition of claim 93, wherein said solubilizing agent is a member selected from polyoxyethanyl-tocopheryl-sebacate (PTS), polyoxyethanyl-sitosterol-sebacate (PSS), polyoxyethanyl-cholesterol-sebacate (PCS), and combinations thereof.

101. The composition of claim 93, wherein said omega-fatty acid is an omega-3-fatty acid; said water-soluble reducing agent is vitamin C; and said solubilizing agent is PTS.
